(19) United States
(12) Patent Application Publication
(10) Pub. No.: US 2005/0037394 A1
(43) Pub. Date: Feb. 17, 2005

(54) Method for in vitro selection of 2'-substituted nucleic acids

(76) Inventors: Anthony D. Keefe, Cambridge, MA (US); Charles Wilson, Concord, MA (US); Paula Burmeister, Cambridge, MA (US); Sara Chesworth Keene, Tewksbury, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/873,856

(22) Filed: Jun. 21, 2004

Related U.S. Application Data
(63) Continuation-in-part of application No. 10/729,581, filed on Dec. 3, 2003.
(60) Provisional application No. 60/430,761, filed on Dec. 3, 2002.
(60) Provisional application No. 60/487,474, filed on Jul. 15, 2003.
(60) Provisional application No. 60/517,039, filed on Nov. 4, 2003.

(51) Int. Cl. C12Q 1/68; C12P 19/34

(52) U.S. Cl. 435/6; 435/91.2

(57) ABSTRACT

Materials and methods are provided for producing aptamer therapeutics having modified nucleotide triphosphates incorporated into their sequence. The aptamers produced by the methods of the invention have increased stability and half life.

### REFERENCE TO RELATED APPLICATIONS

This application is a continuation-in-part of U.S. patent application Ser. No. 10/729,581, filed Dec. 3, 2003, which is related to and claims priority to U.S. Provisional Patent Application Ser. No. 60/430,761, filed Dec. 3, 2002, U.S. Provisional Patent Application Ser. No. 60/487,474, filed Jul. 15, 2003, and U.S. Provisional Patent Application Ser. No. 60/517,039, filed Nov. 4, 2003, each of which is herein incorporated by reference in its entirety.

### FIELD OF THE INVENTION

The invention relates generally to the field of nucleic acids and more particularly to aptamers, and methods for selecting aptamers, incorporating modified nucleotides. The invention further relates to materials and methods for enzymatically producing pools of randomized oligonucleotides having modified nucleotides from which, e.g., aptamers to a specific target can be selected.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (MAbs), are capable of specifically binding to selected targets and, through binding, block their targets' ability to function. Created by an in vitro selection process from pools of random sequence oligonucleotides (FIG. 1), aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (hydrogen bonding, electrostatic complementarity, hydrophobic contacts, steric exclusion, etc) that drive affinity and specificity in antibody-antigen complexes.

Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial (therapeutic) leads. In vitro selection allows the specificity and affinity of the aptamer to be tightly controlled and allows the generation of leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).

3) Administration. Whereas all currently approved antibody therapeutics are administered by intravenous infusion (typically over 2-4 hours), aptamers can be administered by subcutaneous injection. This difference is primarily due to the comparatively low solubility and thus large volumes necessary for most therapeutic MAbs. With good solubility (>150 mg/ml) and comparatively low molecular weight (aptamer: 10-50 kDa; antibody: 150 kDa), a weekly dose of aptamer may be delivered by injection in a volume of less than 0.5 ml. Aptamer bioavailability via subcutaneous administration is >80% in monkey studies (Tucker et al., J. Chromatography B. 732: 203-12, 1999). In addition, the small size of aptamers allows them to penetrate into areas of conformational constrictions that do not allow for antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale synthesizer can produce upwards of 100 kg oligonucleotide per year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$ 100/g in five years.

5) Stability. Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

Given the advantages of aptamers as therapeutic agents, it would be beneficial to have materials and methods to prolong or increase the stability of aptamer therapeutics in vivo. The present invention provides materials and methods to meet these and other needs.

### BRIEF DESCRIPTION OF THE DRAWINGS

FIG. 1 is a schematic representation of the in vitro aptamer selection (SELEX™) process from pools of random sequence oligonucleotides.

FIG. 2 shows a 2′-O-methyl (2′-OMe) modified nucleotide, where “B” is a purine or pyrimidine base.

FIG. 3A is a graph of VEGF-binding by three 2′-OMe VEGF aptamers: ARC224, ARC245 and ARC259; FIG. 3B shows the sequences and putative secondary structures of these aptamers.

FIG. 4 is a graph of the VEGF-binding by various 2′-OH G variants of ARC224 and ARC225

FIG. 5 is a graph of ARC224 binding to VEGF in HUVEC.

FIG. 6 is a graph of ARC224 binding to VEGF before and after autoclaving, in the presence or absence of EDTA.

FIGS. 7A and 7B are graphs of the stability of ARC224 and ARC226, respectively, when incubated at 37° C. in rat plasma.

FIG. 8 is a graph of dRmY SELEX™ Round 6 sequences binding to IgE.

FIG. 9 is a graph of dRmY SELEX™ Round 6 sequences binding to thrombin.

FIG. 10 is a graph of dRmY SELEX™ Round 6 sequences binding to VEGF.

FIG. 11A is a degradation plot of an all 2′-OMe oligonucleotide with 3′-idT, in 95% rat plasma (citrated) at 37° C., and FIG. 11B is a degradation plot of the corresponding dRmY oligonucleotide in 95% rat plasma at 37° C.

FIG. 12 is a graph of rGmH h-IgE binding clones (Round 6).

FIG. 13A is a graph of round 12 pools for rRmY pool PDGF-BB selection, and FIG. 13B is a graph of Round 10 pools for rGmH pool PDGF-BB selection.

FIG. 14 is a graph of dRmY SELEX™ Round 6, 7, 8 and unselected sequences binding to IL-23.

FIG. 15 is a graph of dRmY SELEX™ Round 6, 7 and unselected sequences binding to PDGF-BB.

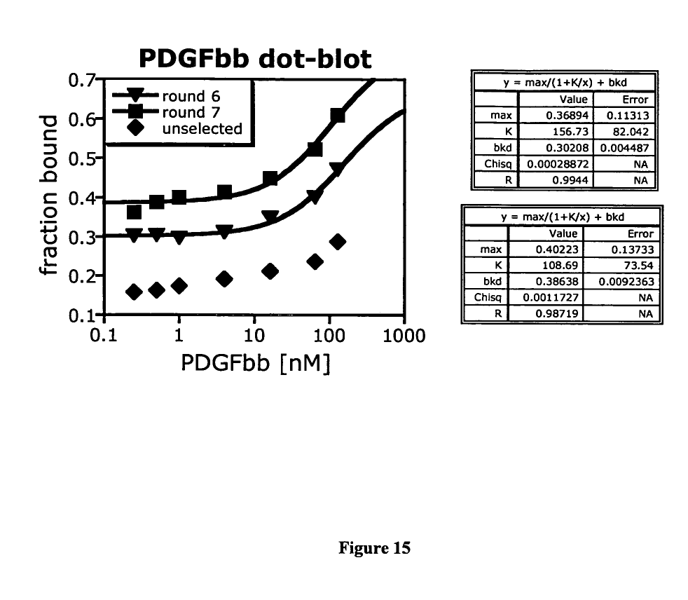

FIG. 16 is a graph depicting the dissociation constants for C5 selection pools. Dissociation constants (K_{d}s) were estimated by fitting the data to the equation: fraction RNA bound=amplitude*K_{d}/(K_{d}+[C5]). “ARC520” refers to the naïve unselected dRmY pool and the “+” indicates the presence of competitor (0.1 mg/ml tRNA, 0.1 mg/ml salmon sperm DNA).

FIG. 17 is a graph depicting C5 clone dissociation constant curves. Dissociation constants (K_{d}s) were estimated by fitting the data to the equation: fraction RNA bound amplitude*K_{d}/(K_{d}+[C5]).

FIG. 18 is a graph depicting an IC₅₀ curve illustrating the inhibitory effect on hemolysis activity of varying concentrations of C5 aptamer clone AMX.221.E1 as compared to ARC186 (anti-C5 aptamer, positive control).

FIG. 19 is a graph depicting pool binding to hIFN-γ. Dissociation constants (K_{d}'s) were estimated fitting the data to the equation: fraction RNA bound=amplitude/(1+K_{d}[hIFN-γ])+background.

FIG. 20 is a graph depicting the binding of clones from Round 10 and Round 12 to hIFN-γ in a 2 point screen (20 nM and 100 nM) using a sandwich filter binding assay.

FIG. 21 is a graph depicting an IC₅₀ curve illustrating the inhibitory effect of ARC789, ARC818, ARC819, and ARC821 on IFN-γ binding to IFN-γ-RI in the IFN-γ ELISA.

### SUMMARY OF THE INVENTION

The present invention provides materials and methods to produce oligonucleotides of increased stability by transcription under the conditions specified herein which promote the incorporation of modified nucleotides into the oligonucleotide. These modified oligonucleotides can be, for example, aptamers, antisense molecules, RNAi molecules, siRNA molecules, or ribozymes. Preferably, the oligonucleotide is an aptamer.

In one embodiment, the present invention provides an improved SELEX™ method (“2′-OMe SELEX™”) that uses randomized pools of oligonucleotides incorporating modified nucleotides from which aptamers to a specific target can be selected.

In one embodiment, the present invention provides methods that use modified enzymes to incorporate modified nucleotides into oligonucleotides under a given set of transcription conditions.

In one embodiment, the present invention provides methods that use a mutated polymerase. In one embodiment, the mutated polymerase is a T7 RNA polymerase. In one embodiment, a T7 RNA polymerase modified by having a mutation at position 639 (from a tyrosine residue to a phenylalanine residue “Y639F”) and at position 784 (from a histidine residue to an alanine residue “H784A”) is used in various transcription reaction conditions which result in the incorporation of modified nucleotides into the oligonucleotides of the invention.

In another embodiment, a T7 RNA polymerase modified with a mutation at position 639 (from a tyrosine residue to a phenylalanine residue) is used in various transcription reaction conditions which result in the incorporation of modified nucleotides into the oligonucleotides of the invention.

In another embodiment, a T7 RNA polymerase modified with a mutation at position 784 (from a histidine residue to an alanine residue) is used in various transcription reaction conditions which result in the incorporation of modified nucleotides into the aptamers of the invention.

In one embodiment, the present invention provides various transcription reaction mixtures that increase the incorporation of modified nucleotides by the modified enzymes of the invention.

In one embodiment, manganese ions are added to the transcription reaction mixture to increase the incorporation of modified nucleotides by the modified enzymes of the invention.

In another embodiment, 2′-OH GTP is added to the transcription mixture to increase the incorporation of modified nucleotides by the modified enzymes of the invention.

In another embodiment, polyethylene glycol, PEG, is added to the transcription mixture to increase the incorporation of modified nucleotides by the modified enzymes of the invention.

In another embodiment, GMP (or any substituted guanosine) is added to the transcription mixture to increase the incorporation of modified nucleotides by the modified enzymes of the invention.

In one embodiment, a leader sequence incorporated into the 5′ end of the fixed region (preferably 20-25 nucleotides in length) at the 5′ end of a template oligonucleotide is used to increase the incorporation of modified nucleotides by the modified enzymes of the invention. Preferably, the leader sequence is greater than about 10 nucleotides in length.

In one embodiment, a leader sequence that is composed of up to 100% (inclusive) purine nucleotides is used.

In another embodiment, a leader sequence at least 6 nucleotides long that is composed of up to 100% (inclusive) purine nucleotides is used.

In another embodiment, a leader sequence at least 8 nucleotides long that is composed of up to 100% (inclusive) purine nucleotides is used.

In another embodiment, a leader sequence at least 10 nucleotides long that is composed of up to 100% (inclusive) purine nucleotides is used.

In another embodiment, a leader sequence at least 12 nucleotides long that is composed of up to 100% (inclusive) purine nucleotides is used.

In another embodiment, a leader sequence at least 14 nucleotides long that is composed of up to 100% (inclusive) purine nucleotides is used.

In one embodiment, the present invention provides aptamer therapeutics having modified nucleotides incorporated into their sequence.

In one embodiment, the present invention provides for the use of aptamer therapeutics having modified nucleotides incorporated into their sequence.

In one embodiment, the present invention provides various compositions of nucleotides for transcription for the selection of aptamers with the SELEX™ process. In one embodiment, the present invention provides combinations of 2′-OH, 2′-F, 2′-deoxy, and 2′-OMe modifications of the ATP, GTP, CTP, TTP, and UTP nucleotides. In another embodiment, the present invention provides combinations of 2′-OH, 2′-F, 2′-deoxy, 2′-OMe, 2′-NH₂, and 2′-methoxyethyl modifications of the ATP, GTP, CTP, TTP, and UTP nucleotides. In one embodiment, the present invention provides 5⁶ combinations of 2′-OH, 2′-F, 2′-deoxy, 2′-OMe, 2′-NH₂, and 2′-methoxyethyl modifications the ATP, GTP, CTP, TTP, and UTP nucleotides.

The invention relates to a method for identifying nucleic acid ligands to a target molecule, where the ligands include modified nucleotides, by: a) preparing a transcription reaction mixture comprising a mutated polymerase, one or more 2′-modified nucleotide triphosphates (NTPs), magnesium ions and one or more oligonucleotide transcription templates; b) preparing a candidate mixture of single-stranded nucleic acids by transcribing the one or more oligonucleotide transcription templates under conditions whereby the mutated polymerase incorporates at least one of the one or more modified nucleotides into each nucleic acid of the candidate mixture, wherein each nucleic acid of the candidate mixture comprises a 2′-modified nucleotide selected from the group consisting of a 2′-position modified pyrimidine and a 2′-position modified purine; c) contacting the candidate mixture with the target molecule; d) partitioning the nucleic acids having an increased affinity to the target molecule relative to the candidate mixture from the remainder of the candidate mixture; and e) amplifying the increased affinity nucleic acids, in vitro, to yield a ligand-enriched mixture of nucleic acids.

The 2′-position modified pyrimidines and 2′-position modified purines include 2′-OH, 2′-deoxy, 2′-O-methyl, 2′-NH₂,2′-F, and 2′-methoxy ethyl modifications. Preferably, the 2′-modified nucleotides are 2′-O-methyl or 2′-F nucleotides.

In some embodiments, the mutated polymerase is a mutated T7 RNA polymerase, such as a T7 RNA polymerase having a mutation at position 639 from a tyrosine residue to a phenylalanine residue (Y639F); a T7 RNA polymerase having a mutation at position 784 from a histidine residue to an alanine residue (H784A); a T7 RNA polymerase having a mutation at position 639 from a tyrosine residue to a phenylalanine residue and a mutation at position 784 from a histidine residue to an alanine residue (Y639F/H784A).

In some embodiments, the oligonucleotide transcription template includes a leader sequence incorporated into the 5′ end of a fixed region at the 5′ end of the oligonucleotide transcription template. The leader sequence, for example, is an all-purine leader sequence. The leader sequence, for example, can be at least 6 nucleotides long; at least 8 nucleotides long; at least 10 nucleotides long; at least 12 nucleotides long; or at least 14 nucleotides long.

In some embodiments, the transcription reaction mixture also includes manganese ions. For example, the concentration of magnesium ions is between 3.0 and 3.5 times greater than the concentration of manganese ions.

In some embodiments of the transcription reaction mixture, each NTP is present at a concentration of 0.5 mM, the concentration of magnesium ions is 5.0 mM, and the concentration of manganese ions is 1.5 mM. In other embodiments of the transcription reaction mixture each NTP is present at a concentration of 1.0 mM, the concentration of magnesium ions is 6.5 mM, and the concentration of manganese ions is 2.0 mM. In other embodiments of the transcription reaction mixture each NTP is present at a concentration of 2.0 mM, the concentration of magnesium ions is 9.6 mM, and the concentration of manganese ions is 2.9 mM.

In some embodiments, the transcription reaction mixture also includes 2′-OH GTP.

In some embodiments, the transcription reaction mixture also includes a polyalkylene glycol. The polyalkylene glycol can be, e.g., polyethylene glycol (PEG).

In some embodiments, the transcription reaction mixture also includes GMP.

In some embodiments, the method for identifying nucleic acid ligands to a target molecule further includes repeating steps d) partitioning the nucleic acids having an increased affinity to the target molecule relative to the candidate mixture from the remainder of the candidate mixture; and e) amplifying the increased affinity nucleic acids, in vitro, to yield a ligand-enriched mixture of nucleic acids.

In some aspects, the invention relates to a nucleic acid ligand to thrombin which was identified according to the method of the invention.

In some aspects, the invention relates to a nucleic acid ligand to vascular endothelial growth factor (VEGF) which was identified according to the method of the invention.

In some aspects, the invention relates to a nucleic acid ligand to IgE which was identified according to the method of the invention.

In some aspects, the invention relates to a nucleic acid ligand to IL-23 which was identified according to the method of the invention.

In some aspects, the invention relates to a nucleic acid ligand to platelet-derived growth factor-BB (PDGF-BB) which was identified according to the method of the invention.

In some embodiments, the transcription reaction mixture includes 2′-OH adenosine triphosphate (ATP), 2′-OH guanosine triphosphate (GTP), 2′-O-methyl cytidine triphosphate (CTP) and 2′-O-methyl uridine triphosphate (UTP).

In some embodiments, the transcription reaction mixture includes 2′-deoxy purine nucleotide triphosphates and 2′-O-methylpyrimidine nucleotide triphosphates.

In some embodiments, the transcription reaction mixture includes 2′-O-methyl adenosine triphosphate (ATP), 2′-OH guanosine triphosphate (GTP), 2′-O-methyl cytidine triphosphate (CTP) and 2′-O-methyl uridine triphosphate (UTP).

In some embodiments, the transcription reaction mixture includes 2′-O-methyl adenosine triphosphate (ATP), 2′-O-methyl cytidine triphosphate (CTP) and 2′-O-methyl uridine triphosphate (UTP), 2′-O-methyl guanosine triphosphate (GTP) and deoxy guanosine triphosphate (GTP), wherein the deoxy guanosine triphosphate comprises a maximum of 10% of the total guanosine triphosphate population.

In some embodiments, the transcription reaction mixture includes 2′-O-methyl adenosine triphosphate (ATP), 2′-F guanosine triphosphate (GTP), 2′-O-methyl cytidine triphosphate (CTP) and 2′-O-methyl uridine triphosphate (UTP).

In some embodiments, the transcription reaction mixture includes 2′-deoxy adenosine triphosphate (ATP), 2′-O-methyl guanosine triphosphate (GTP), 2′-O-methyl cytidine triphosphate (CTP)and 2′-O-methyl uridine triphosphate (UTP).

The invention also relates to a method of preparing a nucleic acid comprising one or more modified nucleotides by: preparing a transcription reaction mixture comprising a mutated polymerase, one or more 2′-modified nucleotide triphosphates (NTPs), magnesium ions and one or more oligonucleotide transcription templates; and contacting the one or more oligonucleotide transcription templates with the mutated polymerase under conditions whereby the mutated polymerase incorporates the one or more 2′-modified nucleotides into a nucleic acid transcription product.

2′-position modified pyrimidines and 2′-position modified purines include 2′-OH, 2′-deoxy, 2′-O-methyl, 2′-NH₂,2′-F, and 2′-methoxy ethyl modifications. Preferably, the 2′-modified nucleotides are 2′-O-methyl or 2′-F nucleotides.

In some embodiments, the mutated polymerase is a mutated T7 RNA polymerase, such as a T7 RNA polymerase having a mutation at position 639 from a tyrosine residue to a phenylalanine residue (Y639F); a T7 RNA polymerase having a mutation at position 784 from a histidine residue to an alanine residue (H784A); a T7 RNA polymerase having a mutation at position 639 from a tyrosine residue to a phenylalanine residue and a mutation at position 784 from a histidine residue to an alanine residue (Y639F/H784A).

In some embodiments, the oligonucleotide transcription template includes a leader sequence incorporated into the 5′ end of a fixed region at the 5′ end of the oligonucleotide transcription template. The leader sequence, for example, is an all-purine leader sequence. The leader sequence, for example, can be at least 6 nucleotides long; at least 8 nucleotides long; at least 10 nucleotides long; at least 12 nucleotides long; or at least 14 nucleotides long.

In some embodiments, the transcription reaction mixture also includes manganese ions. For example, the concentration of magnesium ions is between 3.0 and 3.5 times greater than the concentration of manganese ions.

In some embodiments of the transcription reaction mixture, each NTP is present at a concentration of 0.5 mM, the concentration of magnesium ions is 5.0 mM, and the concentration of manganese ions is 1.5 mM. In other embodiments of the transcription reaction mixture each NTP is present at a concentration of 1.0 mM, the concentration of magnesium ions is 6.5 mM, and the concentration of manganese ions is 2.0 mM. In other embodiments of the transcription reaction mixture each NTP is present at a concentration of 2.0 mM, the concentration of magnesium ions is 9.6 mM, and the concentration of manganese ions is 2.9 mM.

In some embodiments, the transcription reaction mixture also includes 2′-OH GTP.

In some embodiments, the transcription reaction mixture also includes a polyalkylene glycol. The polyalkylene glycol can be, e.g., polyethylene glycol (PEG).

In some embodiments, the transcription reaction mixture also includes GMP.

The invention also relates to an aptamer composition comprising a sequence where substantially all adenosine nucleotides are 2′-OH adenosine, substantially all guanosine nucleotides are 2′-OH guanosine, substantially all cytidine nucleotides are 2′-O-methyl cytidine, and substantially all uridine nucleotides are 2′-O-methyl uridine. In one embodiment, the aptamer has a sequence composition where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In another embodiment, the aptamer has a sequence composition where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine. In another embodiment, the aptamer has a sequence composition where 100% of all adenosine nucleotides are 2′-OH adenosine, at 100% of all guanosine nucleotides are 2′-OH guanosine, 100% of all cytidine nucleotides are 2′-O-methyl cytidine and 100% of all uridine nucleotides are 2′-O-methyl uridine.

The invention also relates to an aptamer composition comprising a sequence where substantially all purine nucleotides are 2′-deoxy purines and substantially all pyrimidine nucleotides are 2′-O-methyl pyrimidines. In one embodiment, the aptamer has a sequence composition where at least 80% of all purine nucleotides are 2′-deoxy purines and at least 80% of all pyrimidine nucleotides are 2′-O-methyl pyrimidines. In another embodiment, the aptamer has a sequence composition where at least 90% of all purine nucleotides are 2′-deoxy purines and at least 90% of all pyrimidine nucleotides are 2′-O-methyl pyrimidines. In another embodiment, the aptamer has a sequence composition where 100% of all purine nucleotides are 2′-deoxy purines and 100% of all pyrimidine nucleotides are 2′-O-methyl pyrimidines.

The invention also relates to an aptamer composition comprising a sequence where substantially all guanosine nucleotides are 2′-OH guanosine, substantially all cytidine nucleotides are 2′-O-methyl cytidine, substantially all uridine nucleotides are 2′-O-methyl uridine, and substantially all adenosine nucleotides are 2′-O-methyl adenosine. In one embodiment, the aptamer has a sequence composition where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In another embodiment, the aptamer has a sequence composition where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In another embodiment, the aptamer has a sequence composition where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

The invention also relates to an aptamer composition comprising a sequence where substantially all adenosine nucleotides are 2′-O-methyl adenosine, substantially all cytidine nucleotides are 2′-O-methyl cytidine, substantially all guanosine nucleotides are 2′-O-methyl guanosine or deoxy guanosine, substantially all uridine nucleotides are 2′-O-methyl uridine, where less than about 10% of the guanosine nucleotides are deoxy guanosine. In one embodiment, the aptamer has a sequence composition where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are deoxy guanosine. In another embodiment, the aptamer has a sequence composition where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are deoxy guanosine. In another embodiment, the aptamer has a sequence composition where 100% of all adenosine nucleotides are 2′-O-methyl adenosine, 100% of all cytidine nucleotides are 2′-O-methyl cytidine, at least 90% of all guanosine nucleotides are 2′-O-methyl guanosine, 100% of all uridine nucleotides are 2′-O-methyl uridine, and no more than about 10% of all guanosine nucleotides are deoxy guanosine.

The invention also relates to an aptamer composition comprising a sequence where substantially all adenosine nucleotides are 2′-O-methyl adenosine, substantially all uridine nucleotides are 2′-O-methyl uridine, substantially all cytidine nucleotides are 2′-O-methyl cytidine, and substantially all guanosine nucleotides are 2′-F guanosine sequence. In one embodiment, the aptamer has a sequence composition where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In another embodiment, the aptamer has a sequence composition where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. In another embodiment, the aptamer has a sequence composition where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

The invention also relates to an aptamer composition comprising a sequence where substantially all adenosine nucleotides are 2′-deoxy adenosine, substantially all cytidine nucleotides are 2′-O-methyl cytidine, substantially all guanosine nucleotides are 2′-O-methyl guanosine, and substantially all uridine nucleotides are 2′-O-methyl uridine. In one embodiment, the aptamer has a sequence composition where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In another embodiment, the aptamer has a sequence composition where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In another embodiment, the aptamer has a sequence composition where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

The invention also relates to an aptamer composition comprising a sequence where substantially all adenosine nucleotides are 2′-OH adenosine, substantially all guanosine nucleotides are 2′-OH guanosine, substantially all cytidine nucleotides are 2′-OH cytidine, and substantially all uridine nucleotides are 2′-OH uridine. In one embodiment, the aptamer has a sequence composition where at least 80% of all adenosine nucleotides are 2′-OH adenosine, at least 80% of all cytidine nucleotides are 2′-OH cytidine, at least 80% of all guanosine nucleotides are 2′-OH guanosine, and at least 80% of all uridine nucleotides are 2′-OH uridine. In another embodiment, the aptamer has a sequence composition where at least 90% of all adenosine nucleotides are 2′-OH adenosine, at least 90% of all cytidine nucleotides are 2′-OH cytidine, at least 90% of all guanosine nucleotides are 2′-OH guanosine, and at least 90% of all uridine nucleotides are 2′-OH uridine. In another embodiment, the aptamer has a sequence composition where 100% of all adenosine nucleotides are 2′-OH adenosine, 100% of all cytidine nucleotides are 2′-OH cytidine, 100% of all guanosine nucleotides are 2′-OH guanosine, and 100% of all uridine nucleotides are 2′-OH uridine.

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

Modified Nucleotide Transcription

The present invention provides materials and methods to produce stabilized oligonucleotides (including, e.g., aptamers) that contain modified nucleotides (e.g., nucleotides which have a modification at the 2′position) which make the oligonucleotide more stable than the unmodified oligonucleotide. The stabilized oligonucleotides produced by the materials and methods of the present invention are also more stable to enzymatic and chemical degradation as well as thermal and physical degradation.

In order for an aptamer to be suitable for use as a therapeutic, it is preferably inexpensive to synthesize, safe and stable in vivo. Wild-type RNA and DNA aptamers are typically not stable in vivo because of their susceptibility to degradation by nucleases. Resistance to nuclease degradation can be greatly increased by the incorporation of modifying groups at the 2′-position. Fluoro and amino groups have been successfully incorporated into oligonucleotide libraries from which aptamers have been subsequently selected. However, these modifications greatly increase the cost of synthesis of the resultant aptamer, and may introduce safety concerns because of the possibility that the modified nucleotides could be recycled into host DNA, by degradation of the modified oligonucleotides and subsequent use of the nucleotides as substrates for DNA synthesis.

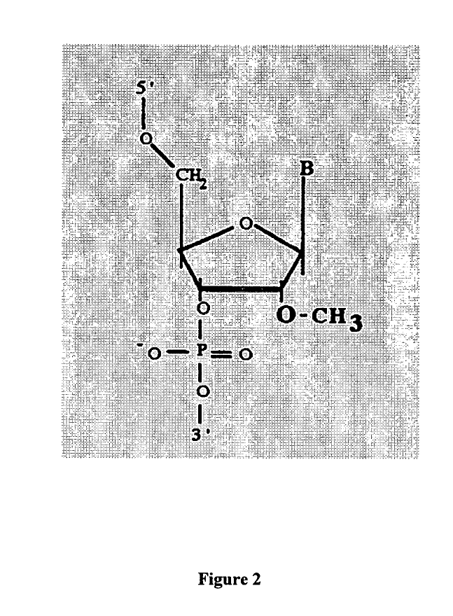

Aptamers that contain 2′-O-methyl (2′-OMe) nucleotides overcome many of these drawbacks. Oligonucleotides containing 2′-O-methyl nucleotides are nuclease-resistant and inexpensive to synthesize. Although 2′-O-methyl nucleotides are ubiquitous in biological systems, natural polymerases do not accept 2′-O-methyl NTPs as substrates under physiological conditions, thus there are no safety concerns over the recycling of 2′-O-methyl nucleotides into host DNA. A generic formula for a 2′-OMe nucleotide is shown in FIG. 2.

There are several examples of 2′-OMe containing aptamers in the literature, see, for example Green et al., Current Biology 2, 683-695, 1995. These were generated by the in vitro selection of libraries of modified transcripts in which the C and U residues were 2′-fluoro (2′-F) substituted and the A and G residues were 2′-OH. Once functional sequences were identified then each A and G residue was tested for tolerance to 2′-OMe substitution, and the aptamer was re-synthesized having all A and G residues which tolerated 2′-OMe substitution as 2′-OMe residues. Most of the A and G residues of aptamers generated in this two-step fashion tolerate substitution with 2′-OMe residues, although, on average, approximately 20% do not. Consequently, aptamers generated using this method tend to contain from two to four 2′-OH residues, and stability and cost of synthesis are compromised as a result. By incorporating modified nucleotides into the transcription reaction which generate stabilized oligonucleotides used in oligonucleotide libraries from which aptamers are selected and enriched by SELEX™ (and/or any of its variations and improvements, including those described below), the methods of the current invention eliminate the need for stabilizing the selected aptamer oligonucleotides (e.g., by resynthesizing the aptamer oligonucleotides with modified nucleotides).

Furthermore, the modified oligonucleotides of the invention can be further stabilized after the selection process has been completed. (See “post-SELEX™ modifications”, including truncating, deleting and modification, below.)

The SELEX™ Method

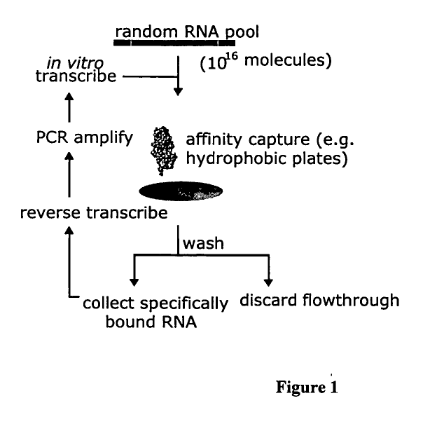

A suitable method for generating an aptamer is with the process entitled “Systematic Evolution of Ligands by EXponential enrichment ” (“SELEX™ ”) depicted generally in FIG. 1. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

SELEX™ relies as a starting point upon a large library of single stranded oligonucleotide templates comprising randomized sequences derived from chemical synthesis on a standard DNA synthesizer. In some examples, a population of 100% random oligonucleotides is screened. In others, each oligonucleotide in the population comprises a random sequence and at least one fixed sequence at its 5′ and/or 3′ end which comprises a sequence shared by all the molecules of the oligonucleotide population. Fixed sequences include sequences such as hybridization sites for PCR primers, promoter sequences for RNA polymerases (e.g., T3, T4, T7, SP6, and the like), restriction sites, or homopolymeric sequences, such as poly A or poly T tracts, catalytic cores, sites for selective binding to affinity columns, and other sequences to facilitate cloning and/or sequencing of an oligonucleotide of interest.

The random sequence portion of the oligonucleotide can be of any length and can comprise ribonucleotides and/or deoxyribonucleotides and can include modified or non-natural nucleotides or nucleotide analogs. See, e.g., U.S. Pat. Nos. 5,958,691; 5,660,985; 5,958,691; 5,698,687; 5,817,635; and 5,672,695, and PCT publication WO 92/07065. Random oligonucleotides can be synthesized from phosphodiester-linked nucleotides using solid phase oligonucleotide synthesis techniques well known in the art (Froehler et al., Nucl. Acid Res. 14:5399-5467 (1986); Froehler et al., Tet. Lett. 27:5575-5578 (1986)). Oligonucleotides can also be synthesized using solution phase methods such as triester synthesis methods (Sood et al., Nucl. Acid Res. 4:2557 (1977); Hirose et al., Tet. Lett., 28:2449 (1978)). Typical syntheses carried out on automated DNA synthesis equipment yield 10¹⁵-10¹⁷ molecules. Sufficiently large regions of random sequence in the sequence design increases the likelihood that each synthesized molecule is likely to represent a unique sequence.

To synthesize randomized sequences, mixtures of all four nucleotides are added at each nucleotide addition step during the synthesis process, allowing for random incorporation of nucleotides. In one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

Template molecules typically contain fixed 5′ and 3′ terminal sequences which flank an internal region of 30-50 random nucleotides. A standard (1 μmole) scale synthesis will yield 10¹⁵-10¹⁶ individual template molecules, sufficient for most SELEX™ experiments. The RNA library is generated from this starting library by in vitro transcription using recombinant T7 RNA polymerase. This library is then mixed with the target under conditions favorable for binding and subjected to step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX™ method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

Within a nucleic acid mixture containing a large number of possible sequences and structures, there is a wide range of binding affinities for a given target. A nucleic acid mixture comprising, for example a 20 nucleotide randomized segment containing only natural unmodified nucleotides can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection and amplification are repeated until a desired goal is achieved. In the most general case, selection/amplification is continued until no significant improvement in binding strength is achieved on repetition of the cycle. The method may be used to sample as many as about 10¹⁸ different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/amplification iterations.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20-50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photoreactive groups capable of binding and/or photocrosslinking to and/or photoinactivating a target molecule. U.S. Pat. No. 5,567,588 and U.S. application Ser. No. 08/792,075, filed Jan. 31, 1997, entitled “Flow Cell SELEX™”, describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules including proteins (including both nucleic acid-binding proteins and proteins not known to bind nucleic acids as part of their biological function) cofactors and other small molecules. For example, see U.S. Pat. No. 5,580,737 which discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of a) preparing a candidate mixture of nucleic acids; b) contacting the candidate mixture with the target, wherein nucleic acids having an increased affinity to the target relative to the candidate mixture may be partitioned from the remainder of the candidate mixture; c) partitioning the increased affinity nucleic acids from the remainder of the candidate mixture; d) contacting the increased affinity nucleic acids with one or more non-target molecules such that nucleic acid ligands with specific affinity for the non-target molecule(s) are removed; and e) amplifying the nucleic acids with specific affinity to the target molecule to yield a mixture of nucleic acids enriched for nucleic acid sequences with a relatively higher affinity and specificity for binding to the target molecule.

One potential problem encountered in the use of nucleic acids as therapeutics and vaccines is that oligonucleotides in their phosphodiester form may be quickly degraded in body fluids by intracellular and/or extracellular enzymes such as endonucleases and exonucleases before the desired effect is manifest. SELEX™ methods therefore encompass the identification of high-affinity nucleic acid ligands which are altered, after selection, to contain modified nucleotides which confer improved characteristics on the ligand, such as improved in vivo stability or improved delivery characteristics. Modifications of nucleic acid ligands include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications include chemical substitutions at the ribose and/or phosphate and/or base positions, such as 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping.

In oligonucleotides which comprise modified sugar groups, for example, one or more of the hydroxyl groups is replaced with halogen, aliphatic groups, or functionalized as ethers or amines. Examples of substitution at the 2′-posititution of the furanose residue include O-alkyl (e.g, O-methyl), O-allyl, S-alkyl, S-allyl, or a halo group. Methods of synthesis of 2′-modified sugars are described in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art.

SELEX™ identified nucleic acid ligands synthesized after selection to contain modified nucleotides are described in U.S. Pat. No. 5,660,985, which describes oligonucleotides containing nucleotide derivatives chemically modified at the 5′ and 2′ positions of pyrimidines. Additionally, U.S. Pat. No. 5,756,703 describes oligonucleotides containing various 2′-modified pyrimidines; and U.S. Pat. No. 5,580,737 describes highly specific nucleic acid ligands containing one or more nucleotides modified with 2′-amino (2′-NH₂), 2′-fluoro (2′-F), and/or 2′-O-methyl (2′-OMe) substituents.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described in U.S. Pat. No. 6,011,020. VEGF nucleic acid ligands that are associated with a lipophilic compound, such as diacyl glycerol or dialkyl glycerol, in a diagnostic or therapeutic complex are described in U.S. Pat. No. 5,859,228.

VEGF nucleic acid ligands that are associated with a lipophilic compound, such as a glycerol lipid, or a non-immunogenic high molecular weight compound, such as polyalkylene glycol are further described in U.S. Pat. No. 6,051,698. VEGF nucleic acid ligands that are associated with a non-immunogenic, high molecular weight compound or a lipophilic compound are further described in PCT Publication No. WO 98/18480. These patents and applications describe the combination of a broad array of oligonucleotide shapes and other properties, and the efficient amplification and replication properties, of oligonucleotides with the desirable properties of other molecules.

The identification of nucleic acid ligands to small, flexible peptides via the SELEX™ method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

To generate oligonucleotide populations which are resistant to nucleases and hydrolysis, modified oligonucleotides can be used and can include one or more substitute internucleotide linkages, altered sugars, altered bases, or combinations thereof. In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotide through an —O—, —N—, or —S— linkage. Not all linkages in the oligonucleotide are required to be identical.

Nucleic acid aptamer molecules are generally selected in a 5 to 20 cycle procedure. In one embodiment, heterogeneity is introduced only in the initial selection stages and does not occur throughout the replicating process.

The starting library of DNA sequences is generated by automated chemical synthesis on a DNA synthesizer. This library of sequences is transcribed in vitro into RNA using T7 RNA polymerase or a modified T7 RNA polymerase, and purified. In one example, the 5′-fixed:random:3′-fixed sequence includes a random sequence having from 30 to 50 nucleotides.

Incorporation of modified nucleotides into the aptamers of the invention is accomplished before (pre-) the selection process (e.g., a pre-SELEX™ process modification). Optionally, aptamers of the invention in which modified nucleotides have been incorporated by pre-SELEX™)ₘ process modification can be further modified by post-SELEX™ process modification (ie., a post-SELEX™ process modification after a pre-SELEX™ modification). Pre-SELEX™ process modifications yield modified nucleic acid ligands with specificity for the SELEX™ target and also improved in vivo stability. Post-SELEX™ process modifications (e.g., modification of previously identified ligands having nucleotides incorporated by pre-SELEX™ process modification) can result in a further improvement of in vivo stability without adversely affecting the binding capacity of the nucleic acid ligand having nucleotides incorporated by pre-SELEX™ process modification.

Modified Polymerases

A single mutant T7 polymerase (Y639F) in which the tyrosine residue at position 639 has been changed to phenylalanine readily utilizes 2′deoxy, 2′amino-, and 2′fluoro-nucleotide triphosphates (NTPs) as substrates and has been widely used to synthesize modified RNAs for a variety of applications. However, this mutant T7 polymerase reportedly can not readily utilize (e.g., incorporate) NTPs with bulkier 2′-substituents, such as 2′-O-methyl (2′-OMe) or 2′-azido (2′-N₃) substituents. For incorporation of bulky 2′ substituents, a double T7 polymerase mutant (Y639F/H784A) having the histidine at position 784 changed to an alanine, or other small amino acid, residue, in addition to the Y639F mutation has been described and has been used to incorporate modified pyrimidine NTPs. A single mutant T7 polymerase (H784A) having the histidine at position 784 changed to an alanine residue has also been described. (Padilla et al., Nucleic Acids Research, 2002, 30: 138). In both the Y639F/H784A double mutant and H784A single mutant T7 polymerases, the change to smaller amino acid residues allows for the incorporation of bulkier nucleotide substrates, e.g., 2′-O methyl substituted nucleotides.

The present invention provides methods and conditions for using these and other modified T7 polymerases having a higher incorporation rate of modified nucleotides having bulky substituents at the furanose 2′ position, than wild-type polymerases. Generally, it has been found that under the conditions disclosed herein, the Y693F single mutant can be used for the incorporation of all 2′-OMe substituted NTPs except GTP and the Y639F/H784A double mutant can be used for the incorporation of all 2′-OMe substituted NTPs including GTP. It is expected that the H784A single mutant possesses similar properties when used under the conditions disclosed herein.

The present invention provides methods and conditions for modified T7 polymerases to enzymatically incorporate modified nucleotides into oligonucleotides. Such oligonucleotides may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or libraries of transcripts are generated using any combination of modifications, for example, ribonucleotides, (2′-OH, “rN”), deoxyribonucleotides (2′-deoxy), 2′-F, and 2′-OMe nucleotides. A mixture containing 2′-OMe C and U and 2′-OH A and G is called “rRmY”; a mixture containing deoxy A and G and 2′-OMe U and C is called “dRmY”; a mixture containing 2′-OMe A, C, and U, and 2′-OH G is called “rGmH”; a mixture alternately containing 2′-OMe A, C, U and G and 2′-OMe A, U and C and 2′-F G is called “toggle”; a mixture containing 2′-OMe A, U, C, and G, where up to 10% of the G's are deoxy is called “r/mGmH”; a mixture containing 2′-O Me A, U, and C, and 2′-F G is called “fGmH”; and a mixture containing deoxy A, and 2′-OMe C, G and U is called “dAmB”.

A preferred embodiment includes any combination of 2′-OH, 2′-deoxy and 2′-OMe nucleotides. A more preferred embodiment includes any combination of 2′-deoxy and 2′-OMe nucleotides. An even more preferred embodiment is with any combination of 2′-deoxy and 2′-OMe nucleotides in which the pyrimidines are 2′-OMe (such as dRmY, mN or dGmH).

2′-Modified SELEX™

The present invention provides methods to generate libraries of 2′-modified (e.g., 2′-OMe) RNA transcripts in conditions under which a polymerase accepts 2′-modified NTPs. Preferably, the polymerase is the Y693F/H784A double mutant or the Y693F single mutant. Other polymerases, particularly those that exhibit a high tolerance for bulky 2′-substituents, may also be used in the present invention. Such polymerases can be screened for this capability by assaying their ability to incorporate modified nucleotides under the transcription conditions disclosed herein. A number of factors have been determined to be crucial for the transcription conditions useful in the methods disclosed herein. For example, great increases in the yields of modified transcript are observed when a leader sequence is incorporated into the 5′ end of a fixed sequence at the 5′ end of the DNA transcription template, such that at least about the first 6 residues of the resultant transcript are all purines.

Another important factor in obtaining transcripts incorporating modified nucleotides is the presence or concentration of 2′-OH GTP. Transcription can be divided into two phases: the first phase is initiation, during which an NTP is added to the 3′-hydroxyl end of GTP (or another substituted guanosine) to yield a dinucleotide which is then extended by about 10-12 nucleotides, the second phase is elongation, during which transcription proceeds beyond the addition of the first about 10-12 nucleotides. It has been found that small amounts of 2′-OH GTP added to a transcription mixture containing an excess of 2′-OMe GTP are sufficient to enable the polymerase to initiate transcription using 2′-OH GTP, but once transcription enters the elongation phase the reduced discrimination between 2′-OMe and 2′-OH GTP, and the excess of 2′-OMe GTP over 2′-OH GTP allows the incorporation of principally the 2′-OMe GTP.

Another important factor in the incorporation of 2′-OMe into transcripts is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions. To obtain the greatest yields of maximally 2′ substituted O-methylated transcripts (i e., all A, C, and U and about 90% of G nucleotides), concentrations of approximately 5 mM magnesium chloride and 1.5 mM manganese chloride are preferred when each NTP is present at a concentration of 0.5 mM. When the concentration of each NTP is 1.0 mM, concentrations of approximately 6.5 mM magnesium chloride and 2.0 mM manganese chloride are preferred. When the concentration of each NTP is 2.0 mM, concentrations of approximately 9.6 mM magnesium chloride and 2.9 mM manganese chloride are preferred. In any case, departures from these concentrations of up to two-fold still give significant amounts of modified transcripts.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

For maximum incorporation of 2′-OMe ATP (100%), UTP(100%), CTP(100%) and GTP (˜90%) (“r/mGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (6.5 mM where the concentration of each 2′-OMe NTP is 1.0 mM), MnCl₂ 1.5 mM (2.0 mM where the concentration of each 2′-OMe NTP is 1.0 mM), 2′-OMe NTP (each) 500 μM (more preferably, 1.0 mM), 2′-OH GTP 30 μM, 2′-OH GMP 500 μM, pH 7.5, Y639F/H784A T7 RNA Polymerase 15 units/ml, inorganic pyrophosphatase 5 units/ml, and an all-purine leader sequence of at least 8 nucleotides long. As used herein, one unit of the Y639F/H784A mutant T7 RNA polymerase, or any other mutant T7 RNA polymerase specified herein) is defined as the amount of enzyme required to incorporate 1 nmole of 2′-OMe NTPs into transcripts under the r/mGmH conditions. As used herein, one unit of inorganic pyrophosphatase is defined as the amount of enzyme that will liberate 1.0 mole of inorganic orthophosphate per minute at pH 7.2 and 25° C.

For maximum incorporation (100%) of 2′-OMe ATP, UTP and CTP (“rGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe UTP and CTP (“rRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F/H784A T7 RNA Polymerase 15 units/ml, inorganic pyrophosphatase 5 units/ml, and an all-purine leader sequence of at least 8 nucleotides long.

For maximum incorporation (100%) of deoxy ATP and GTP and 2′-OMe UTP and CTP (“dRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of 2′-OMe ATP, UTP and CTP and 2′-F GTP (“fGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For maximum incorporation (100%) of deoxy ATP and 2′-OMe UTP, GTP and CTP (“dAmB”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/ml, inorganic pyrophosphatase 5 units/ml, and an all-purine leader sequence of at least 8 nucleotides long.

For each of the above, (1) transcription is preferably performed at a temperature of from about 30° C. to about 45° C. and for a period of at least two hours and (2) 50-300 nM of a double stranded DNA transcription template is used (200 nm template was used for round 1 to increase diversity (300 nm template was used for dRmY transcriptions), and for subsequent rounds approximately 50 nM, a 1/10 dilution of an optimized PCR reaction, using conditions described herein, was used). The preferred DNA transcription templates are described below (where ARC254 and ARC256 transcribe under all 2′-OMe conditions and ARC255 transcribes under rRmY conditions).

| | | |
|---|---|---|
| ARC254: | 5′-CATCGATGCTAGTCGTAACGATCCNNNNNNNNNNNNNNNNNNNNNNNNN | (SEQ ID NO:1) |
| | NNNNNCGAGAACGTTCTCTCCTCTCCCTATAGTGAGTCGTATTA-3′ | |
| ARC255: | 5′-CATGCATCGCGACTGACTAGCCGNNNNNNNNNNNNNNNNNNNNNNNNNN | (SEQ ID NO:2) |
| | NNNNGTAGAACGTTCTCTCCTCTCCCTATAGTGAGTCGTATTA-3′ | |
| ARC256: | 5′-CATCGATCGATCGATCGACAGCGNNNNNNNNNNNNNNNNNNNNNNNNNN | (SEQ ID NO:453) |
| | NNNNGTAGAACGTTCTCTCCTCTCCCTATAGTGAGTCGTATTA-3′ | |

Under rN transcription conditions of the present invention, the transcription reaction mixture comprises 2′-OH adenosine triphosphates (ATP), 2′-OH guanosine triphosphates (GTP), 2′-OH cytidine triphosphates (CTP), and 2′-OH uridine triphosphates (UTP). The modified oligonucleotides produced using the rN transcription mixtures of the present invention comprise substantially all 2′-OH adenosine, 2′-OH guanosine, 2′-OH cytidine, and 2′-OH uridine. In a preferred embodiment of rN transcription, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-OH cytidine, and at least 80% of all uridine nucleotides are 2′-OH uridine. In a more preferred embodiment of rN transcription, the resulting modified oligonucleotides of the present invention comprise a sequence where at least 90% of all adenosine nucleotides are 2′-OH adenosine, at least 90% of all guanosine nucleotides are 2′-OH guanosine, at least 90% of all cytidine nucleotides are 2′-OH cytidine, and at least 90% of all uridine nucleotides are 2′-OH uridine. In a most preferred embodiment of rN transcription, the modified oligonucleotides of the present invention comprise 100% of all adenosine nucleotides are 2′-OH adenosine, of all guanosine nucleotides are 2′-OH guanosine, of all cytidine nucleotides are 2′-OH cytidine, and of all uridine nucleotides are 2′-OH uridine.

Under rRmY transcription conditions of the present invention, the transcription reaction mixture comprises 2′-OH adenosine triphosphates, 2′-OH guanosine triphosphates, 2′-O-methyl cytidine triphosphates, and 2′-O-methyl uridine triphosphates. The modified oligonucleotides produced using the rRmY transcription mixtures of the present invention comprise substantially all 2′-OH adenosine, 2′-OH guanosine, 2′-O-methyl cytidine and 2′-O-methyl uridine. In a preferred embodiment, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In a more preferred embodiment, the resulting modified oligonucleotides comprise a sequence where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine In a most preferred embodiment, the resulting modified oligonucleotides comprise a sequence where 100% of all adenosine nucleotides are 2′-OH adenosine, 100% of all guanosine nucleotides are 2′-OH guanosine, 100% of all cytidine nucleotides are 2′-O-methyl cytidine and 100% of all uridine nucleotides are 2′-O-methyl uridine.

Under dRmY transcription conditions of the present invention, the transcription reaction mixture comprises 2′-deoxy purine triphosphates and 2′-O-methylpyrimidine triphosphates. The modified oligonucleotides produced using the dRmY transcription conditions of the present invention comprise substantially all 2′-deoxy purines and 2′-O-methyl pyrimidines. In a preferred embodiment, the resulting modified oligonucleotides of the present invention comprise a sequence where at least 80% of all purine nucleotides are 2′-deoxy purines and at least 80% of all pyrimidine nucleotides are 2′-O-methyl pyrimidines. In a more preferred embodiment, the resulting modified oligonucleotides of the present invention comprise a sequence where at least 90% of all purine nucleotides are 2′-deoxy purines and at least 90% of all pyrimidine nucleotides are 2′-O-methyl pyrimidines. In a most preferred embodiment, the resulting modified oligonucleotides of the present invention comprise a sequence where 100% of all purine nucleotides are 2′-deoxy purines and 100% of all pyrimidine nucleotides are 2′-O-methyl pyrimidines.

Under rGmH transcription conditions of the present invention, the transcription reaction mixture comprises 2′-OH guanosine triphosphates, 2′-O-methyl cytidine triphosphates, 2′-O-methyl uridine triphosphates, and 2′-O-methyl adenosine triphosphates. The modified oligonucleotides produced using the rGmH transcription mixtures of the present invention comprise substantially all 2′-OH guanosine, 2′-O-methyl cytidine, 2′-O-methyl uridine, and 2′-O-methyl adenosine. In a preferred embodiment, the resulting modified oligonucleotides comprise a sequence where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In a more preferred embodiment, the resulting modified oligonucleotides comprise a sequence where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In a most preferred embodiment, the resulting modified oligonucleotides comprise a sequence where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

Under r/mGmH transcription conditions of the present invention, the transcription reaction mixture comprises 2′-O-methyl adenosine triphosphate, 2′-O-methyl cytidine triphosphate, 2′-O-methyl guanosine triphosphate, 2′-O-methyl uridine triphosphate and deoxy guanosine triphosphate. The resulting modified oligonucleotides produced using the r/mGmH transcription mixtures of the present invention comprise substantially all 2′-O-methyl adenosine, 2′-O-methyl cytidine, 2′-O-methyl guanosine, and 2′-O-methyl uridine, wherein the population of guanosine nucleotides has a maximum of about 10% deoxy guanosine. In a preferred embodiment, the resulting r/mGmH modified oligonucleotides of the present invention comprise a sequence where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are deoxy guanosine. In a more preferred embodiment, the resulting modified oligonucleotides comprise a sequence where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are deoxy guanosine. In a most preferred embodiment, the resulting modified oligonucleotides comprise a sequence where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine, and no more than about 10% of all guanosine nucleotides are deoxy guanosine.

Under fGmH transcription conditions of the present invention, the transcription reaction mixture comprises 2′-O-methyl adenosine triphosphates (ATP), 2′-O-methyl uridine triphosphates (UTP), 2′-O-methyl cytidine triphosphates (CTP), and 2′-F guanosine triphosphates. The modified oligonucleotides produced using the fGmH transcription conditions of the present invention comprise substantially all 2′-O-methyl adenosine, 2′-O-methyl uridine, 2′-O-methyl cytidine, and 2′-F guanosine. In a preferred embodiment, the resulting modified oligonucleotides comprise a sequence where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In a more preferred embodiment, the resulting modified oligonucleotides comprise a sequence where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. The resulting modified oligonucleotides comprise a sequence where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

Under dAmB transcription conditions of the present invention, the transcription reaction mixture comprises 2′-deoxy adenosine triphosphates (dATP), 2′-O-methyl cytidine triphosphates (CTP), 2′-O-methyl guanosine triphosphates (GTP), and 2′-O-methyl uridine triphosphates (UTP). The modified oligonucleotides produced using the dAmB transcription mixtures of the present invention comprise substantially all 2′-deoxy adenosine, 2′-O-methyl cytidine, 2′-O-methyl guanosine, and 2′-O-methyl uridine. In a preferred embodiment, the resulting modified oligonucleotides comprise a sequence where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In a more preferred embodiment, the resulting modified oligonucleotides comprise a sequence where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

In each case, the transcription products can then be used as the library in the SELEX™ process to identify aptamers and/or to determine a conserved motif of sequences that have binding specificity to a given target. The resulting sequences are already stabilized, eliminating this step from the process to arrive at a stabilized aptamer sequence and giving a more highly stabilized aptamer as a result. Another advantage of the 2′-OMe SELEX™ process is that the resulting sequences are likely to have fewer 2′-OH nucleotides required in the sequence, possibly none.

As described below, lower but still useful yields of transcripts fully incorporating 2′-OMe substituted nucleotides can be obtained under conditions other than the optimized conditions described above. For example, variations to the above transcription conditions include:

The HEPES buffer concentration can range from 0 to 1 M. The present invention also contemplates the use of other buffering agents having a pKa between 5 and 10, for example without limitation, Tris(hydroxymethyl)aminomethane.

The DTT concentration can range from 0 to 400 mM. The methods of the present invention also provide for the use of other reducing agents, for example without limitation, mercaptoethanol.

The spermidine and/or spermine concentration can range from 0 to 20 mM.

The PEG-8000 concentration can range from 0 to 50% (w/v). The methods of the present invention also provide for the use of other hydrophilic polymer, for example without limitation, other molecular weight PEG or other polyalkylene glycols.

The Triton X-100 concentration can range from 0 to 0.1% (w/v). The methods of the present invention also provide for the use of other non-ionic detergents, for example without limitation, other detergents, including other Triton-X detergents.

The MgCl₂ concentration can range from 0.5 mM to 50 mM. The MnCl₂ concentration can range from 0.15 mM to 15 mM. Both MgCl₂ and MnCl₂ must be present within the ranges described and in a preferred embodiment are present in about a 10 to about 3 ratio of MgCl₂:MnCl₂, preferably, the ratio is about 3-5, more preferably, the ratio is about 3 to about 4.

The 2′-OMe NTP concentration (each NTP) can range from 5 μM to 5 mM.

The 2′-OH GTP concentration can range from 0 μM to 300 μM.

The 2′-OH GMP concentration can range from 0 to 5 mM.

The pH can range from pH 6 to pH 9. The methods of the present invention can be practiced within the pH range of activity of most polymerases that incorporate modified nucleotides.

In addition, the methods of the present invention provide for the optional use of chelating agents in the transcription reaction condition, for example without limitation, EDTA, EGTA, and DTT.

Pharmaceutical Compositions

The invention also includes pharmaceutical compositions containing the aptamer molecules described herein. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. Compositions of the invention are useful for administration to a subject suffering from, or predisposed to, a disease or disorder which is related to or derived from a target to which the aptamers specifically bind.

For example, the target is a protein involved with a pathology, for example, the target protein causes the pathology.

Compositions of the invention can be used in a method for treating a patient having a pathology. The method involves administering to the patient a composition comprising aptamers that bind a target (e.g., a protein) involved with the pathology, so that binding of the composition to the target alters the biological function of the target, thereby treating the pathology.

The patient having a pathology, e.g. the patient treated by the methods of this invention can be a mammal, or more particularly, a human.

In practice, the compounds or their pharmaceutically acceptable salts, are administered in amounts which will be sufficient to exert their desired biological activity.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixirs, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine, oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 1000 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Effective plasma levels of the compounds of the present invention range from 0.002 mg to 50 mg per kg of body weight per day.

Compounds of the present invention may be administered in a single daily dose, or the total daily dosage may be administered in divided doses of two, three or four times daily.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same.

The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### 2′-OMe SELEX™ Against Thrombin and VEGF targets

A library of approximately 3×10¹⁴ unique transcription templates, each containing a random region of thirty contiguous nucleotides, was synthesized as described below, and PCR amplified. Cloning and sequencing of this library demonstrated that the composition of the random region in this library was approximately 25% of each nucleotide. The DNA library was purified away from unincorporated dNTPs by gel-filtration and ethanol-precipitation. Modified transcripts were then generated from a mixture containing 500 uM of each of the four 2′-OMe NTPs, i.e., A, C, U and G, and 30 uM 2′-OH GTP (“r/mGmH”). In addition, modified transcripts were generated from mixtures containing part modified nucleotides and part ribonucleotides or all ribonucleotides namely, a mixture containing all 2′-OH nucleotides (rN); a mixture containing 2′-OMe C and U and 2′-OH A and G (rRmY); a mixture containing 2′-OMe A, C, and U, and 2′-OH G (“rGmH”); and a mixture alternately containing 2′-OMe A, C, U and G and 2′-OMe A, U and C and 2′-F G (“toggle”). These modified transcripts were then used in SELEX™ against targets—e.g., VEGF and thrombin.

Generally, after gel-purification and DNase-treatment these modified transcripts were dissolved in PBS for VEGF or 1×ASB (150 mM KCl, 20 mM HEPES, 10 mM MgCl₂, 1 mM DTT, 0.05% Tween20, pH 7.4) for thrombin, and incubated for one hour in an empty well on a hydrophobic multiwell plate to subtract plastic-binding sequences. The supernatant was then transferred to a well that had previously been incubated for one hour at room temperature in PBS for VEGF or in ASBND (150 mM KCl, 20 mM HEPES, 10 mM MgCl₂, 1 mM DTT, pH 7.4) for thrombin. After a one hour incubation the well was washed and bound sequences were reverse-transcribed in situ using thermoscript reverse transcriptase (Invitrogen) at 65° C. for one hour. The resultant cDNA was then PCR-amplified, separated from dNTPs by gel-filtration, and used to generate modified transcripts for input into the next round of selection. After 10 rounds of selection and amplification the ability of the resultant library to bind to VEGF or thrombin was assessed by Dot-Blot. At this point, the library was cloned, sequenced and individual clones were assayed for their ability to bind VEGF or thrombin. Using this combination of sequence and clonal binding data, sequence motifs were identified.

One VEGF aptamer motif, exemplified by ARC224, which was common to both the r/mGmH and toggle selections, was used to design smaller synthetic constructs which were also assayed for binding to VEGF and ultimately minimized aptamers to VEGF were identified, ARC245 and ARC259, both of which are 23 nucleotides long. Another VEGF aptamer motif, exemplified by ARC226, which was common to all 2′-OMe selections, was also identified. The ARC224 aptamer produced by the methods of the present invention has the sequence 5′-mCmGmAmUmAmUmGmCmAmGmUmUmUmGmAmGmAmAmGmUmCmGmCmGmC mAmUmUmCmG-3T (SEQ ID No. 184) where “m” represents a 2′-O-methyl substitution.

The ARC226 aptamer has the sequence: 5-mGmAmUmCmAmUmGmCmAmUGmUmGmGmAmUmCmGmCmGmGmAmUmC-[3T]-3′ (SEQ ID No. 186).

The ARC245 aptamer has sequence:

| | |
|---|---|
| 5′-mAmUmGmCmAmGmUmUmUmGmAmGmAmAmGmUmCmGmCmGmCmAmU-[3T]-3′. | (SEQ ID No. 187) |

The ARC259 aptamer has the sequence:

| | |
|---|---|
| 5′-mAmCmGmCmAmGmUmUmUmGmAmGmAmAmGmUmCmGmCmGmCmGMu-[3T]-3′. | (SEQ ID No. 188) |

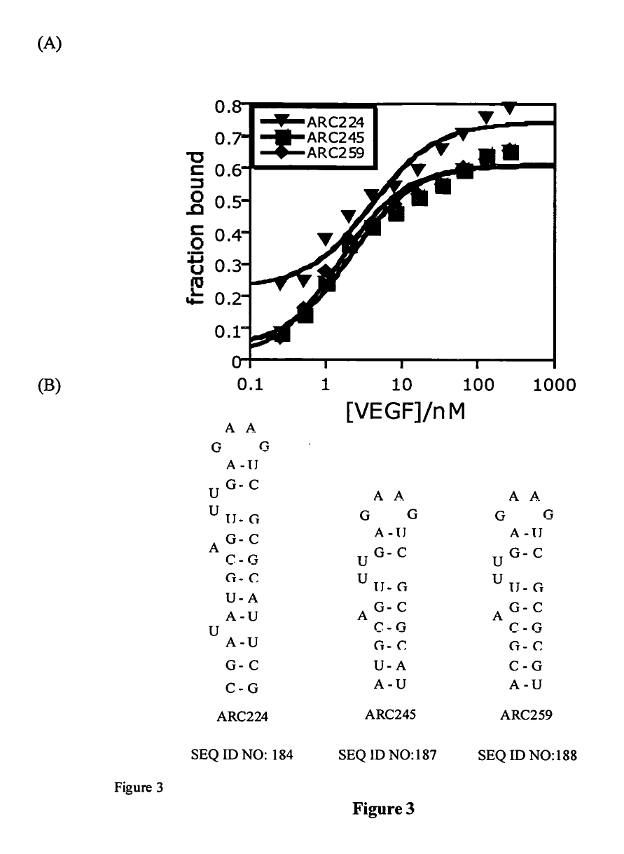

FIG. 3A is a graph of VEGF binding by ARC224, ARC245 and ARC259. A schematic representation of the secondary structure of these aptamers is presented in FIG. 3B.

All residues in ARC224, ARC226 and ARC245 are 2′-OMe and all constructs (initially identified by SELEX™) were generated by solid-phase chemical synthesis. The K_{D} values of these aptamers, determined by dot-blot in PBS, are as follows: ARC224 3.9 nM, ARC245 2.1 nM, ARC259 1.4 nM.

Reagents. All reagents were acquired from Sigma (St. Louis, Mo.) except where otherwise stated.

Oligonucleotide synthesis. DNA syntheses were undertaken according to standard protocols using an Expedite 8909 DNA synthesizer (Applied Biosystems, Foster City, Calif.). The DNA library used in this study had the following sequence: ARC254: 5′-CATCGATGCTAGTCGTAACGATCCNNNNNNNNNNNNNNNNNNNNNNNNNNNNN NCGAGAACGTTCTCTCCTCTCCCTATAGTGAGTCGTATTA-3′ (SEQ ID NO: 1) in which each N has an equal probability of being each of the four nucleotides. 2′-OMe RNA syntheses, including those containing 2′-OH nucleotides, were undertaken according to standard protocols using a 3900 DNA Synthesizer (Applied Biosystems, Foster City, Calif.). All oligonucleotides were purified by denaturing PAGE except PCR and RT primers.

2′-OMe Library Generation. The synthetic DNA library (1.5 nmol) was amplified by PCR under standard conditions with the following primers: 3′-primer

| | |
|---|---|
| 5′-CATCGATGCTAGTCGTAACGATCC-3′ | (SEQ ID NO:454) and 5′-primer |
| 5′-TAATACGACTCACTATAGGGAGAGGAGAGAAACGTTCTCG-3′. | (SEQ ID NO:455) |

The resultant library of double-stranded transcription templates was precipitated and separated from unincorporated nucleotides by gel-filtration. At no point was the library denatured, either by thermal means or by exposure to low-salt conditions. r/mGmH transcription was performed under the following conditions to produce template for the first round of selection: double-stranded DNA template 200 nM, HEPES 200 mM, DTT 40 mM, Triton X-100 0.01%, Spermidine 2 mM, 2′-O-methyl ATP, CTP, GTP and UTP 500 μM each, 2′-OH GTP 30 μM, GMP 500 μM, MgCl₂ 5.0 mM, MnCl₂ 1.5 mM, inorganic pyrophosphatase 0.5 units per 100 μL reaction, Y639F/H784A T7 RNA polymerase 1.5 units per 100 μl reaction pH 7.5 and 10% w/v PEG and were incubated at 37° C. overnight. The resultant transcripts were purified by denaturing 10% PAGE, eluted from the gel, incubated with RQ1 DNase (Promega, Madison Wis.), phenol-extracted, chloroform-extracted, precipitated and taken up in PBS. For the initiation of selection transcripts were additionally generated by the direct chemical synthesis of 2′-OMe RNA, these were purified by denaturing 10% polyacrylamide gel electrophoresis, eluted from the gel and taken up in PBS.

For the rN, rRmY and rGmH transcriptions, the transcription conditions were as follows, where 1×Tc buffer is: 200 mM HEPES, 40 mM DTT, 2 mM Spermidine, 0.01% Triton X-100, pH 7.5.

When 2′-OH A, C, U and G (rN) conditions were used, the transcription reaction conditions were MgCl₂ 25 mM, each NTP 5 mM, 1×Tc buffer, 10% w/v PEG, T7 RNA polymerase 1.5 units, and 50-200 nM double stranded template (200 nM of template was used in Round 1 to increase diversity and for subsequent rounds approximately 50 nM, a 1/10 dilution of an optimized PCR reaction using conditions described herein, was used).

When 2′-OMe C and U and 2′-OH A and G (rRmY) conditions were used, the transcription reaction conditions were 1× Tc buffer, 50-200 nM double stranded template (200 nM of template was used in Round 1 to increase diversity and for subsequent rounds approximately 50 nM, a 1/10 dilution of an optimized PCR reaction using conditions described herein, was used), 5.0 mM MgCl₂, 1.5 mM MnCl₂, 0.5 mM each base, 10% PEG-8000, 0.25 units inorganic pyrophosphatase, and 1.5 units Y639F/H784A T7 RNA polymerase.

When 2′-OMe A, C, and U and 2′-OH G (rGmH) conditions were used, the transcription reaction conditions were 1× Tc buffer, 50-200 nM double stranded DNA template (200 nM of template was used in Round 1 to increase diversity for subsequent rounds approximately 50 nM, a 1/10 dilution of an optimized PCR reaction using conditions described herein, was used), 5.0 mM MgCl₂, 1.5 mM MnCl₂, 0.5 mM each base, 10% PEG-8000, 0.25 units inorganic pyrophosphatase, and 1.5 units Y639F single mutant T7 RNA polymerase in 100 μl volume.

When 2′-OMe A, C, U and 2′-F G conditions were used, the transcription reaction conditions were as for rGmH, except 0.5 mM 2′-F GTP is used instead of 2′-OH GTP.

Reverse Transcription. The reverse transcription conditions used during SELEX™ are as follows (100 μL reaction volume): 1× Thermo buffer (Invitrogen), 4 μM primer, 10 mM DTT, 0.2 mM each DNTP, 200 μM Vanadate nucleotide inhibitor, 10 μg/ml tRNA, Thermoscript RT enzyme 1.5 units (Invitrogen). Reverse transcriptase reaction yields are lower for 2′-OMe templates. PCR reaction conditions are as follows 1× ThermoPol buffer (NEB), 0.5 μM 5′ primer, 0.5 μM 3′ primer 0.2 mM each DHTP, Taq DNA Polymerase 5 units (NEB).

2′-OMe SELEX™ Protocol. As noted above, SELEX™ was performed with the modified transcripts against each of two targets (VEGF and Thrombin) using 5 kinds of transcripts for a total of 10 selections. The five kinds of transcripts were: “rN” (all 2′-OH), “rRmY” (2′-OH A, G, 2′-OMe C, U), “rGmH” (2′-OH G, 2′-OMe C, U, A), “r/mGmH” (2′-OMe A, U, G, C 500 uM, 2′-OH G 30 uM), “toggle” (alternately “r/mGmH” and 2′-OMe A, U, C, 2′-F G).

All of the selections directed against VEGF generated VEGF specific aptamers while only the rN and rRmY selections against thrombin generated thrombin specific aptamers. The aptamer sequences identified in these selections are set forth in Tables 1 through 5 (VEGF) and Tables 6 through 10 (thrombin) below.

The sequences are from SELEX™ round 11 except for Thrombin “rGmH”, “r/mGmH” and “toggle” which are from round 5, VEGF “r/mGmH” which is from round 10 and VEGF “toggle” which is from round 8.

The selection was performed by initially immobilizing the protein by hydrophobic absorption to “NUNC MAXY” plates, washing away the protein that didn't bind, incubating the library of 2′-OMe-substituted transcripts with the immobilized protein, washing away the transcripts that didn't bind, performing RT directly in the plate, then PCR, and then transcribing the resultant double-stranded DNA template under the appropriate transcription conditions.

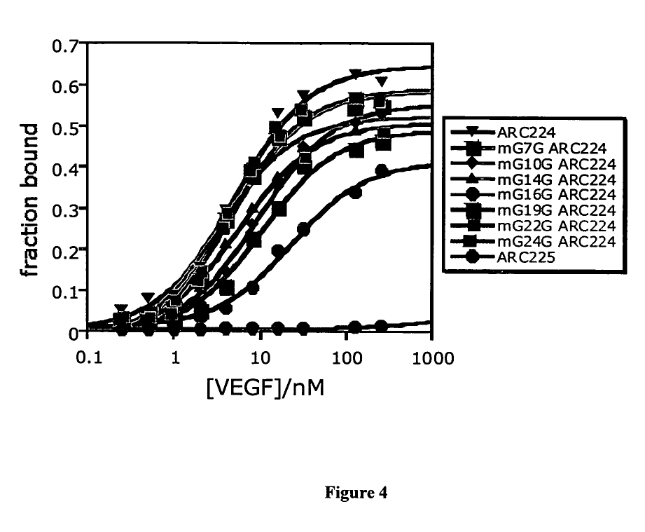

Binding assays were performed with trace ³²P-body-labelled transcripts that were incubated with various protein concentrations in silanized wells, these were then passed through a sandwich of a nitrocellulose membrane over a nylon membrane. Protein-bound RNA is visualized on the NC membrane, unbound RNA on the nylon membrane. The proportion binding is then used to calculate affinity (see FIGS. 4, 5, and 6). For example, the binding characteristics of various 2′-OH G variants of ARC224 (all 2-OMe) are shown in FIG. 4. The nomenclature “mGXG” indicates a substitution of 2′-OH G for 2′-OMe G at position “X”, as numbered sequentially from the 5′-terminus. Thus, mG7G ARC224 is ARC224 with a 2′-OH at position 7. ARC225 is ARC224 with 2′-OMe to 2′-OH substitutions at positions 7, 10, 14, 16, 19, 22 and 24. All constructs (initially identified by SELEX™) were generated by solid-phase chemical synthesis. These data were generated by dot-blot in PBS. The fully 2′-OMe aptamer, ARC224, has superior VEGF-binding characteristics when compared to any of the 2′-OH substituted variants studied.

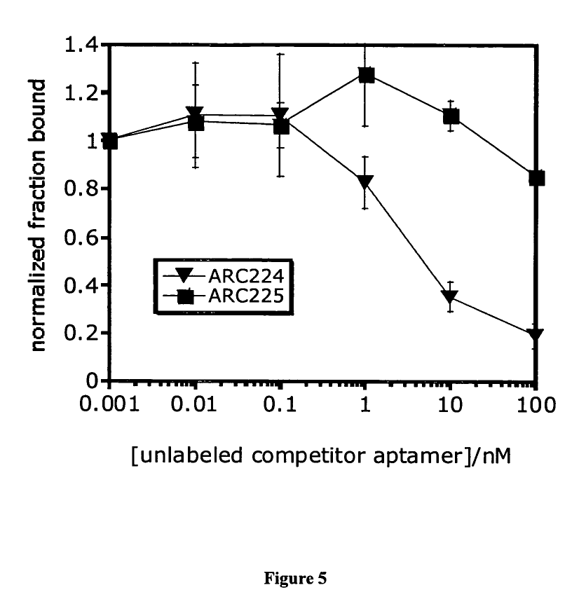

FIG. 5 is a plot of ARC224 and ARC225 binding to VEGF. This graph indicates that ARC224 binds VEGF in a manner which inhibits the biological function of VEGF. ¹²⁵I-labeled VEGF was incubated with the aptamer and this mixture was then incubated with human umbilical cord vascular endothelial cells (HUVEC). The supernatant was removed, the cells were washed, and bound VEGF was counted in a scintillation counter. ARC225 has the same sequence as ARC224 and 2′-OMe to 2′-OH substitutions at positions 7, 10, 14, 16, 19, 22 and 24 numbered from the 5′-terminus. These data indicate that the IC₅₀ of ARC224 is approximately 2 nM.

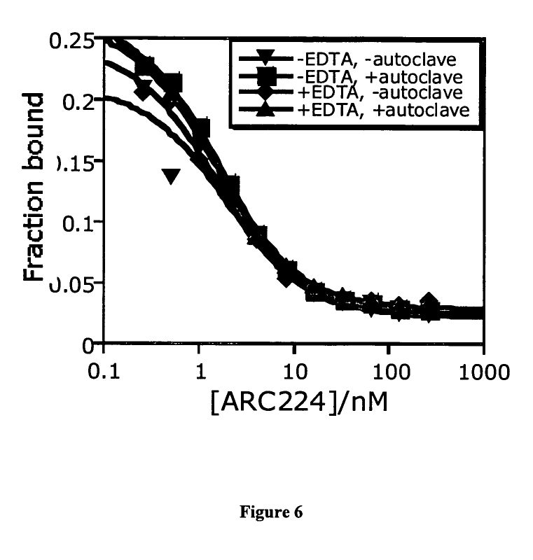

FIG. 6 is a binding curve plot of ARC224 binding to VEGF before and after autoclaving, with or without EDTA. FIG. 6 shows both the proportion of aptamer that is functional and the IC₅₀ for binding to VEGF before and after autoclaving for 25 minutes with a peak temperature of 125° C. These data were determined by the inhibition by unlabeled ARC224 of the binding of 5′-labeled ARC224 to 1 nM VEGF in PBS as measured by dot-blot in PBS. Where indicated, samples contained 1 mM EDTA. All constructs (initially identified by SELEX™) were generated by solid-phase chemical synthesis. No degradation of ARC224 was observed within the limitations of this assay.

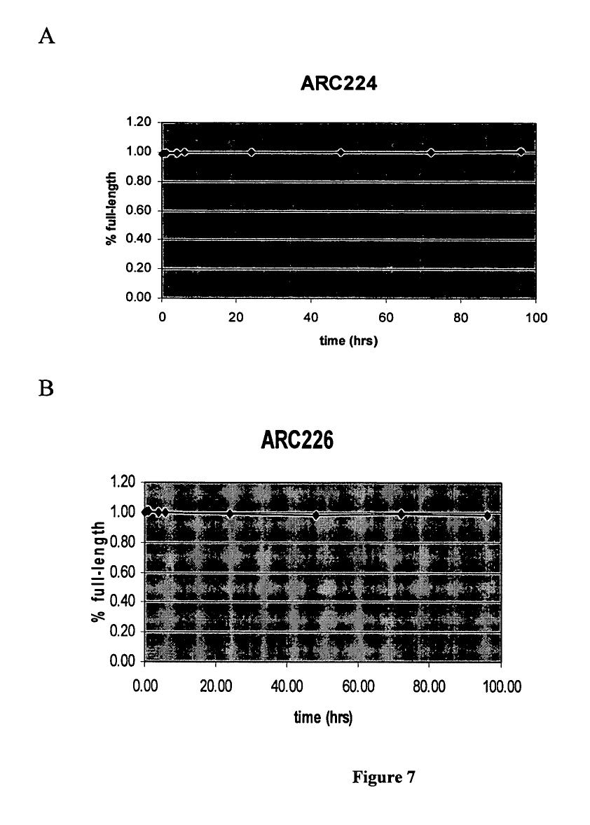

Degradation studies show that incubation in plasma at 37° C. over 4 days induces so little degradation that measuring a half-life is not possible, but is at least in excess of 4 days (see, e.g., FIG. 7). FIGS. 7A and 7B are plots of the stability of ARC224 and ARC226, respectively, when incubated at 37° C. in rat plasma. As indicated in the figure, both ARC224 and ACR226 showed no detectable degradation after for 4 days in rat plasma. In these experiments, 5′-labeled ARC224 and ARC226 were incubated in rat plasma at 37° C. and analyzed by denaturing PAGE. All constructs (initially identified by SELEX™) were generated by solid-phase chemical synthesis. The half-life appears to be in excess of 100 hours.

Tables 1 through Table 10 below show the DNA sequences of aptamers corresponding to the transcribed aptamers isolated from the various libraries, i e. rN, rRmY, rGmH, and r/mGmH, as indicated. The sequence of the aptamers will have uridine residues instead of thymidine residues in the DNA sequences shown. Table 11 shows the stabilized aptamer sequences obtained by the methods of the present invention. As used herein, “3T”, refers to an inverted thymidine nucleotide attached to the oligonucleotide phosphodiester backbone at the 5′ position, the resulting oligo having two 5′-OH ends and is thus resistant to 3′ nucleases.

Unless noted otherwise, individual sequences listed in the various tables represent the cDNA clones of the aptamers that were selected under the SELEX™ conditions provided. The actual aptamers provided in the invention are those corresponding sequences comprising the rN, in N, rRmY, rGmH, r/mGmH, dRmY and toggle combinations of residues, as indicated in the text.

### 2′-OMe SELEX™ Results.

TABLE 1
Corresponding cDNAs of the VEGF Aptamer Sequences-all 2′-OH (rN)

| | |
|---|---|
| SEQ ID No. 3 | > PB.97.126.F_43-H1 |

GGGAGAGGAGAGAACGTTCTCGAAATGATGCATGTTCGTAAAATGGCAGTATTGGATCGTTACAACTAGCATCGATG

| | |
|---|---|
| SEQ ID No. 4 | > PB.97.126.F_43-A2 |

GGGAGAGGAGAGAACGTTCTCGTGCCGAGGTCCGGAACCTTGATGATTGGCGGGATCGTTACGACTAGCATCGATG

| | |
|---|---|
| SEQ ID No. 5 | > PB.97.126.F_48-A1 |

GGGAGAGGAGAGAACGTTCTCGCATTTGGGCTAGTTGTGAAATGGCAGTATTGGATCGTTACGACTAGCATCGATG

| | |
|---|---|
| SEQ ID No. 6 | > PB.97.126.F_48-B1 |

GGGAGAGGAGAGAACGTTCTCGAATCGTAGATAGTCGTGAAATGGCAGTATTGGATCGTTACGACTAGCATCGATG

| | |
|---|---|
| SEQ ID No. 7 | > PB.97.126.F_48-C1 |

GGGAGAGGAGAGAACGTTCTCGTTCTAGTCGGTACGATATGTTGACGAATCCGGATCGTTACGACTAGCATCGATG

| | |
|---|---|
| SEQ ID No. 8 | > PB.97.126.F_48-D1 |

GGGAGAGGAGAGAACGTTCTCGTTTGATGAGGCGGACATAATCCGTGCCGAGCGGGATCGTTACGACTAGCATCGATG

| | |
|---|---|
| SEQ ID No. 9 | > PB.97.126.F_48-E1 |

GGGAGAGGAGAGAACGTTCTCGAAGGAAAAGAGTTTAGTATTGGCCGTCCGTGGGATCGTTACGACTAGCATCGATG

| | |
|---|---|
| SEQ ID No. 10 | > PB.97.126.F_48-F1 |

GGGAGAGGAGAGAACGTTCTCGTGCCGAGGTCCGGAACCTTGATGATTGGCGGGATCGTTACGACTAGCATCGATG

| | |
|---|---|
| SEQ ID No. 11 | > PB.97.126.F_48-G1 |

GGGAGAGGAGAGAACGTTCTCGTACGGTCCATTGAGTTTGAGATGTCGCCATGGATCGTTACGACTAGCATCGATG

| | |
|---|---|
| SEQ ID No. 12 | > PB.97.126.F_48-B2 |

GGGAGAGGAGAGAACGTTCTCGAGTTAGTGGTAACTGATATGTTGAATTGTCCGGATCGTTACGACTAGCATCGATG

| | |
|---|---|
| SEQ ID No. 13 | > PB.97.126.F_48-C2 |

GGGAGAGGAGAGAACGTTCTCGCACGGATGGCGAGAACAGAGATTGCTAGGTGGATCGTTACGACTAGCATCGATG

| | |
|---|---|
| SEQ ID No. 14 | > PB.97.126.F_48-D2 |

GGGAGAGGAGAGAACGTTCTCGNTANCGNTNCGCCNTGCTAACGCNTANTTGGGATCGTTACGACTAGCATCGATG

| | |
|---|---|
| SEQ ID No. 15 | > PB.97.126.F_48-E2 |

GGGAGAGGAGAGAACGTTCTCGAAGATGAGTTTTGTCGTGAAATGGCAGTATTGGATCGTTACGACTAGCATCGATG

| | |
|---|---|
| SEQ ID No. 16 | > PB.97.126.F_48-F2 |

GGGAGAGGAGAGAACGTTCTCGGGATGCCGGATTGATTTCTGATGGGTACTGGGATCGTTACGACTAGCATCGATG

| | |
|---|---|
| SEQ ID No. 17 | > PB.97.126.F_48-G2 |

GGGAGAGGAGAGAACGTTCTCGAATGGAATGCATGTCCATCGCTAGCATTTGGGATCGTTACGACTAGCATCGATG

| | |
|---|---|
| SEQ ID No. 18 | > PB.97.126.F_48-H2 |

GGGAGAGGAGAGAACGTTCTCGTGCTGAGGTCCGGAACCTTGATGATTGGCGGGATCGTTNCNACTAGCATCGATG

| | |
|---|---|
| SEQ ID No. 19 | > PB.97.126.F_48-A3 |

GGGAGAGGAGAGAACGTTCTCGCTAATTGCTGAGTCGTGAAGTGGCAGTATTGGATCGTTACGACTAGCATCGATG

| | |
|---|---|
| SEQ ID No. 20 | > PB.97.126.F_48-B3 |

GGGAGAGGAGAGAACGTTCTCGTAACGATGTCCGGGGCGAAAGGCTAGCATGGGATCGTTACGACTAGCATCGATG

| | |
|---|---|
| SEQ ID No. 21 | > PB.97.126.F_48-C3 |

GGGAGAGGAGAGAACGTTCTCGATGCGATTGTCGAGATTTGTAAGATAGCTGTGGATCGTTACGACTAGCATCGATG

TABLE 2
Corresponding cDNAs of the VEGF Aptamer Sequences-2′-OH AG, 2′-OMe CU (rRmY)

| | |
|---|---|
| SEQ ID No. 22 | > PB.97.126.G_43-D3 |

GGGAGAGGAGAGAACGTTCTCGCAGAAAACATCTTTGCGGTTGAATACATGTGGATCGTTACGACTAGCATCGATG

| | |
|---|---|
| SEQ ID No. 23 | > PB.97.126.G_43-G3 |

GGGAGAGGAGAGAACGTTCTCGAAAAAAGANANCNNCCTTCNGAATACATGCGGATCGTTACGACTAGCATCGATG

| | |
|---|---|
| SEQ ID No. 24 | > PB.97.126.G_48-E3 |

GGGAGAGGAGAGAACGTTCTCGAGAGTGATTCGATGCTTCANGAATACATGTGGATCGTTACGACTAGCATCGATG

| | |
|---|---|
| SEQ ID No. 25 | > PB.97.126.G_48-F3 |

GGGAGAGGAGAGAACGTTCTCGACANNNCNTNGCTNGGTTGANTACATGTGNNTNTCNNNANCNNTNNTCTNTNANAG
GGG

| | |
|---|---|
| SEQ ID No. 26 | > PB.97.126.G_48-H3 |

GGGAGAGGAGAGAACGTTCTCGAAGAAGGAAAGCTGCAAGTCGAATACACGCGGATCGTTACGACTAGCATCGATG

| | |
|---|---|
| SEQ ID No. 27 | > PB.97.126.G_48-A4 |

GGGAGAGGAGAGAACGTTCTCGCAACATCGATTACAGTTGAGTACATGTGGATCGTTACGACTAGCATTCGATG

| | |
|---|---|
| SEQ ID No. 28 | > PB.97.126.G_48-B4 |

GGGAGAGGAGAGAACGTTCTCGAGACATCATTGCTCGTTGAATACATGTGGATCGTTACGACTAGCATCGATG

| | |
|---|---|
| SEQ ID No. 29 | > PB.97.126.G_48-C4 |

GGGAGAGGAGAGAACGTTCTCGCCAAAGTAGCTTCGACAGTCGAATACATGTGGATCGTTACGACTAGCATCGATG

| | |
|---|---|
| SEQ ID No. 30 | > PB.97.126.G_48-D4 |

GGGAGAGGAGAGAACGTTCTCGAAAATCAGTACTGTGCAGTCGAATACATGCGGATCGTTACGACTAGCATCGATG

| | |
|---|---|
| SEQ ID No. 31 | > PB.97.126.G_48-E4 |

GGAGAGGAGAGAACGTTCTCGTAATGACATCAATGCTTCTTGAATACAGGTGGATCGTTACGACTAGCATCGATG

| | |
|---|---|
| SEQ ID No. 32 | > PB.97.126.G_48-F4 |

GGGAGAGGAGAGAACGTTCTCGAGAAAAACGATCTGTGACGTGTAATCCGCGGATCGTTACGACTAGCATCGATG

| | |
|---|---|
| SEQ ID No. 33 | > PB.97.126.G_48-G4 |

GGGAGAGGAGAGAACGTTCTCGCAACAAACGTCGACGCTTCTGAATACATGTGGATCGTTACGACTAGCATCGATG

| | |
|---|---|
| SEQ ID No. 34 | > PB.97.126.G_48-H4 |

GGGAGAGGAGAGAACGTTCTCGTGATCATAGAAATGCTAGCTGAATACATGTGGATCGTTACGACTAGCATCGATG

| | |
|---|---|
| SEQ ID No. 35 | > PB.97.126.G_48-A5 |

GGGAGAGGAGAGAACGTTCTCGCAGCGTAAAATGCTTTTCGAAGTACATGTGGATCGTTACGACTAGCATCGATG

| | |
|---|---|
| SEQ ID No. 36 | > PB.97.126.G_48-B5 |

GGGAGAGGAGAGAACGTTCTCGCCAAGAATCAATCGCTTGTCGAATACATGCGGATCGTTACGACTAGCATCGATG

| | |
|---|---|
| SEQ ID No. 37 | > PB.97.126.G_48-C5 |

GGGAGAGGAGAGAACGTTCTCGTGATCATAGAAATGCTAGCTGAGTACATGTGGATCGTTACGACTAGCATCGATG

| | |
|---|---|
| SEQ ID No. 38 | > PB.97.126.G_48-D5 |

GGGAGAGGAGAGAACGTTCTCGCAGAAAACATCTTTGCGGTTGAATACATGTGGATCGTTACGACTAGCATCGATG

| | |
|---|---|
| SEQ ID No. 39 | > PB.97.126.G_48-E5 |

GGGAGAGGAGAGAACGTTCTCGNAAACANNCATCTATTGNAGTTGAATACATGTGGATCGTTACGACTAGCATCGATG

| | |
|---|---|
| SEQ ID No. 40 | > PB.97.126.G_48-F5 |

GGGAGAGGAGAGAACGTTCTCGCTAAAGATTCGCTGCTTGCCGAATACATGTGGATCGTTACGACTAGCATCGATG

TABLE 3
Corresponding cDNAs of the VEGF Aptamer Sequences-2′-OH G, 2′-OMe CUA (rGmH)

| | |
|---|---|
| SEQ ID No. 41 | > PB.97.126.H_43-H6 |

GGGAGAGGAGAGAACGTTCTCGGGTTTTGTCTGCGTTTGTGCGTTGAACCCGGGATCGTTACGACTAGCATCGATG

| | |
|---|---|
| SEQ ID No. 42 | > PB.97.126.H_43-F7 |

GGGAGAGGAGAGAACGTTCTCGTGATTACGTGATGAGGATCCGCGTTTTCTCGGGATCGTTACGACTAGCATCGATG

| | |
|---|---|
| SEQ ID No. 43 | > PB.97.126.H_43-H7 |

GGGAGAGGAGAGAACGTTCTCGTTAGTGAAAACGATCATGCATGTGGATCGCGGATCGTTACGACTAGCATCGATG

| | |
|---|---|
| SEQ ID No. 44 | > PB.97.126.H_48-H5 |

GGGAGAGGAGAGAACGTTCTCGTGTTCATTCGTTTGCTTATCGTTGCATGTGGATCGTTACGACTAGCATCGATG

| | |
|---|---|
| SEQ ID No. 45 | > PB.97.126.H_48-A6 |

AGGAGAGGAGAGAACGTTCTCGGCAGAGTGTGATGTGCATCCGCACGTGCCGGGATCGTTACGACTAGCATCGATG

| | |
|---|---|
| SEQ ID No. 46 | > PB.97.126.H_48-B6 |

GGAGAGGAGAGAACGTTCTCGTTAGTATACGATCGTGCATGTGGATCGCGGATCGTTACGACTAGCATCGATG

| | |
|---|---|
| SEQ ID No. 47 | > PB.97.126.H_48-C6 |

GGGAGAGGAGAGAACGCCCCCCTGATTNCGTGAAGAGGATCCGCANTTTCNCGGGATCGTTACGACTAGCATCGATG

| | |
|---|---|
| SEQ ID No. 48 | > PB.97.126.H_48-D6 |

GGAGAGGAGAGAACGTTCTCGTGGCTTTGGAACGGGTACGGATTTGGCACGGGATCGTTACGACTAGCATCGATG

| | |
|---|---|
| SEQ ID No. 49 | > PB.97.126.H_48-E6 |

GGGAGAGGAGAGAACGTTCTCGTGATTACGTGATGAGGATCCGCGTTTTCTCGGGATCGTTACGACTAGCATCGATG

| | |
|---|---|
| SEQ ID No. 50 | > PB.97.126.H_48-F6 |

GGGAGAGGAGAGAACGTTCTCGTCATTGGTGACNGCGTTGCATGTGGATCGCGGATCGTTACGACTAGCATCGATG

| | |
|---|---|
| SEQ ID No. 51 | > PB.97.126.H_48-G6 |

GGGAGAGGAGAGAACGTTCTCGNTGGTNNAANGCTTTTGTNGGGNTANNTGTGGATCGTTACGACTAGCATCGATG

| | |
|---|---|
| SEQ ID No. 52 | > PB.97.126.H_48-A7 |

GGGAGAGGAGAGAACGTTCTCGTGGCTTTGGAACGAATTCGGATTTGGCACGGGATCGTTACGACTAGCATCGATG

| | |
|---|---|
| SEQ ID No. 53 | > PB.97.126.H_48-B7 |

GGGAGAGGAGAGAACGTTCTCGTGCGATGTCGTGGATTTCCGTTTCGCAAGGGATCGTTACGACTAGCATCGATG

| | |
|---|---|
| SEQ ID No. 54 | > PB.97.126.H_48-C7 |

GGGAGAGGAGAGAACGTTCTCGTGAAGCAGATGTCGTTGGCGACTTAGAGGGGGATCGTTACGACTAGCATCGATG

| | |
|---|---|
| SEQ ID No. 55 | > PB.97.126.H_48-D7 |

GGGAGAGGAGAGAACGTTCTCGTGATTTCGTGATGAGGATCCGCGTTTTCTCGGGATCGTTACGACTAGCATCGATG

| | |
|---|---|
| SEQ ID No. 56 | > PB.97.126.H_48-E7 |

GGGAGAGGAGAGAACGTTCTCGCTAGTAACGATGACTTGATGAGCATCCGAGGATCGTTACGACTAGCATCGATG

| | |
|---|---|
| SEQ ID No. 57 | > PB.97.126.H_48-G7 |

GGAGAGGAGAGAACGTTCTCGTCATAAGTAACGACGTTGCATGTGGATCGCGGATCGTTACGACTAGCATCGATG

| | |
|---|---|
| SEQ ID No. 58 | > PB.97.126.H_48-A8 |

GGGAGAGGAGAGAACGTTCTCGCAAGGAGATGGTTGCTAGCTGAGTACATGTGGATCGTTACGACTAGCATCGATG

TABLE 4

Corresponding cDNAs of the VEGF Aptamer Sequences-2′-OMe AUGC (r/mGmH,
each G has a 90% probability of having a 2′-OMe group incorporated
therein)

| | |
|---|---|
| SEQ ID No. 59 | PB.97.126.I_43-B8 |

GGGAGAGGAGAGAACGTTCTCGCGATATGCAGTTTGAGAAGTCGCGCATTCGGGGGATCGTTACGACTAGCATCGATG

| | |
|---|---|
| SEQ ID No. 60 | > PB.97.126.I_48-C8 |

GGGAGAGGAGAGAACGTTCTCGTGCGACGGGCTTCTTGTGTCATTCGCATGGGATCGTTACGACTAGCATCGATG

| | |
|---|---|
| SEQ ID No. 61 | > PB.97.126.I_48-D8 |

GGGAGAGGAGAGAACGTTCTCGGCATTGCAGTTGATAGGTCGCGCAGTGCTGGGATCGTTACGACTAGCATCGATG

| | |
|---|---|
| SEQ ID No. 62 | > PB.97.126.I_48-E8 |

GGGAGAGGAGAGAACGTTCTCGCGATATGCAGTCTGAGAAGTCGCGCATTCGAGGGATCGTTACGACTAGCATCGATG

| | |
|---|---|
| SEQ ID No. 63 | > PB.97.126.I_48-F8 |

GGGAGAGGAGAGAACGTTCTCGTGTAGCAAGCATGTGGATCGCGACTGCACGGGATCGTTACGACTAGCATCGATG

| | |
|---|---|
| SEQ ID No. 64 | > PB.97.126.I_48-G8 |

GGGAGAGGAGAGAACGTTCTCGGATAAGCAGTTGAGATGTCGCGCTTTGACGGGATCGTTACGACTAGCATCGATG

| | |
|---|---|
| SEQ ID No. 65 | > PB.97.126.I_48-H8 |

GGGAGAGGAGAGAACGTTCTCGATGANCANTTTGAGAAGTCGCGCTTGTCGGGATCGTTACGACTAGCATCGATG

| | |
|---|---|
| SEQ ID No. 66 | > PB.97.126.I_48-A9 |

GGGAGAGGAGAGAACGTTCTCGAGTAATGCAGTGGAAGTCGCGCATTACCTGGGATCGTTACGACTAGCATCATG

| | |
|---|---|
| SEQ ID No. 67 | > PB.97.126.I_48-B9 |

GGGAGAGGAGAGAACGTTCTCGCGATATGCAGTTTGAGAAGTCGCGCATTCGGGGGATCGTTACGACTAGCATCGATG

| | |
|---|---|
| SEQ ID No. 68 | > PB.97.126.I_48-C9 |

GGGAGAGGAGAGAACGTTCTCGTGATNCAGTTGANAAGTCNCGCATACAGGATCGTTACGACTAGCATCGATG

| | |
|---|---|
| SEQ ID No. 69 | > PB.97.126.I_48-D9 |

GGGAGAGGAGAGAACGTTCTCGAGTAATGCTGTGGAAGTCGCGCATTTCCTGGGATCGTTACGACTAGCATCGATG

| | |
|---|---|
| SEQ ID No. 70 | > PB.97.126.I_48-D8 |

GGGAGAGGAGAGAACGTTCTCGGCATTGCAGTTGATAGGTCGCGCAGTGCTGGGATCGTTACGACTAGCATCGATG

| | |
|---|---|
| SEQ ID No. 71 | > PB.97.126.I_48-F9 |

GGGAGAGGAGAGAACGTTCTCGCGATATGCAGTTTGGGAAGTCGCGCATTCGAGGGATCGTTACGACTAGCATCGATG

| | |
|---|---|
| SEQ ID No. 72 | > PB.97.126.I_48-G9 |

GGGAGAGGAGAGAACGTTCTCGCNATATGCTGTTTGANAANTCGCGCATTCGGGGGATCGTTACGACTAGCATCGATG

| | |
|---|---|
| SEQ ID No. 73 | > PB.97.126.I_48-H9 |

GGGAGAGGAGAGAACGTTCTCGCGTAGATTGGGCTGAATGGGATATCTTTAGCGGGATCGTTACGACTAGCATCGATG

| | |
|---|---|
| SEQ ID No. 74 | > PB.97.126.I_48-B10 |

GGGAGAGGAGAGAACGTTCTCGCGATATGCAGTTTGAGAAGTCGCGCTTTCGAGGGATCGTTACGACTAGCATCGATG

| | |
|---|---|
| SEQ ID No. 75 | > PB.97.126.I_48-D10 |

GGGAGAGGAGAGAACGTTCTCGTCAATCTGATGTAGCCTCACGTGGGCGGAGTCGGATCGTTACGACTAGCATCGATG

TABLE 5

Corresponding cDNAs of the VEGF Aptamer Sequences-alternately “r/mGmH” and
2′-OMe AUC, 2′-F G (toggle)

| | |
|---|---|
| SEQ ID No. 76 | > PB.97.126.J_48-F10 |

GGGAGAGGAGAGAACGTTCTCGGATCGTTACGACTAGCATCGATG

| | |
|---|---|
| SEQ ID No. 77 | > PB.97.126.J_48-G10 |

GGGAGAGGAGAGAACGTTCTCGGATCGTTACGACTAGCATCGATG

| | |
|---|---|
| SEQ ID No. 78 | > PB.97.126.J_48-H10 |

GGGAGAGGAGAGAACGTTCTCGGTGGTGTTGCTGAACTGTCGCGTTTCGCCGGGATCGTTACGACTAGCATCGATG

| | |
|---|---|
| SEQ ID No. 79 | > PB.97.126.J_48-A11 |

GGGAGAGGAGAGAACGTTCTCGTCGCGATTGCATATTTTCCGCCTTGCTGTGAGGATCGTTACGACTAGCATCGATG

| | |
|---|---|
| SEQ ID No. 80 | > PB.97.126.J_48-B11 |

GGGAGAGGAGAGAACGTTCTCGCGATTTGCAGTTTGAGATGTCGCGCATTCGAGGGATCGTTACGACTAGCATCGATG

| | |
|---|---|
| SEQ ID No. 81 | > PB.97.126.J_48-C11 |

GGGAGAGGAGAGAACGTTCTCGCGATATGCAGTTTGAGAAGTCGCGCATTCGGGGGATCGTTACGACTAGCATCGATG

| | |
|---|---|
| SEQ ID No. 82 | > PB.97.126.J_48-D11 |

GGGAGAGGAGAGAACGTTCTCGTTGGTGCAGTTTGAGATGTCGCGCACCTTGGGATCGTTACGACTAGCATCGATG

| | |
|---|---|
| SEQ ID No. 83 | > PB.97.126.J_48-E11 |

GGGAGAGGAGAGAACGTTCTCGGTATTGGTTCCATTAAGCTGGACACTCTGCTCCGGGATCGTTACGACTAGCATCGATG

| | |
|---|---|
| SEQ ID No. 84 | > PB.97.126.J_48-F11 |

GGGAGAGGAGAGAACGTTCTCGTTGGTGCAGTTTGAGATGTCGCGCGCCTTGGGATCGTTACGACTAGCATCGATG

| | |
|---|---|
| SEQ ID No. 85 | > PB.97.126.J_48-G11 |

GGGAGAGGAGAGAACGTTCTCGCGATATGCAGTTTGAGAAGTCGCGCATTCGAGGGATCGTTACNACTAGCATCGATG

| | |
|---|---|
| SEQ ID No. 86 | > PB.97.126.J_48-A12 |

GGGAGAGGAGAGAACGTTCTCGCGATATGCAGTTTGAGAAGTCGCGCATTCGGGGGATCGTTACGACTAGCATCGATG

| | |
|---|---|
| SEQ ID No. 87 | > PB.97.126.J_48-B12 |

GGGAGAGGAGAGAACGCTCTCGGGGACNNAAANNCGAATTGNCGCGTGNGTCCGGGGGAGCGCCCGACTAGTCATCGATG

| | |
|---|---|
| SEQ ID No. 88 | > PB.97.126.J_48-C12 |

GGGAGAGGAGAGAACGTTCTCGCGATATGNANTTTGAGAAGTCGCGCATTCGGGGGATCGTTACGACTAGCATCGATG

| | |
|---|---|
| SEQ ID No. 89 | > PB.97.126.J_48-D12 |

GGGAGAGGAGAGAACGTTCTCGGTGTACAGCTTGAGATGTCGCGTACTCCGGGATCGTTACGACTAGCATCGATG

| | |
|---|---|
| SEQ ID No. 90 | > PB.97.126.J_48-E12 |

GGGAGAGGAGAGAACGTTCTCGCGATATGCAGTTTGAGAAGTCGCGCATTCGGGGGATCGTTACGACTAGCATCGATG

| | |
|---|---|
| SEQ ID No. 91 | > PB.97.126.J_48-F12 |

GGGAGAGGAGAGAACGTTCTCGAGTAAGAAAGCTGAATGGTCGCACTTCTCGGGATCGTTACGACTAGCATCGATG

| | |
|---|---|
| SEQ ID No. 92 | > PB.97.126.J_48-G12 |

AGGGAGAGGAAGAACGTTCTCGCGATGTGCAGTTTGAGAAGTCGCGCATTCGAGGGATCGTTACGACTAGCATCGATG

| | |
|---|---|
| SEQ ID No. 93 | > PB.97.126.J_48-H12 |

GGGAGAGGAGAGAACGTTCTCGAAAGAATCAGCATGCGGATCGCGGCTTTCGGGATCGTTACGACTAGCATCGATG

TABLE 6
Corresponding cDNAs of the Thrombin Aptamer Sequences-all 2′-OH (rN)

| | |
|---|---|
| SEQ ID No. 94 | > PB.97.126.A_44-A1 |

GGGAGAGGAGAGAACGTTCTCGANTCCANTNTNCNTGGAGGAGTAAGTACCTGAGGGATCGTTACGACTAGCATCGATG

| | |
|---|---|
| SEQ ID No. 95 | > PB.97.126.A_44-B1 |

GGGAGAGGAGAGAACGTTCTCGGGAAACAAGGAACTTAGAGTTANTTGACCGGGATCGTTACGACTAGCATCGATG

| | |
|---|---|
| SEQ ID No. 96 | > PB.97.126.A_44-C1 |

GGGAGAGGAGAGAACGTTCTCGTACCATGCAAGGAACATAATAGTTAGCGTGGGATCGTTACGACTAGCATCGATG

| | |
|---|---|
| SEQ ID No. 97 | > PB.97.126.A_44-D1 |

GGGAGAGGAGAGAACGTTCTCGGGACACAAGGAACACAATAGTTAGTGTACGGGATCGTTACGACTAGCATCGATG

| | |
|---|---|
| SEQ ID No. 98 | > PB.97.126.A_44-E1 |

GGGAGAGGAGAGAACGTTCTCGTCTGCAAGGAACACAATAGTTAGCATTGCGGGATCGTTACGACTAGCATCGATG

| | |
|---|---|
| SEQ ID No. 99 | > PB.97.126.A_44-F1 |

GGGAGAGGAGAGAACGTTCTCGCGCCAACAAAGCTGGAGTACTTAGAGCGCGGGATCGTTACGACTAGCATCGATG

| | |
|---|---|
| SEQ ID No. 100 | > PB.97.126.A_44-G1 |

GGGAGAGGAGAGAACGTTCTCGATTGCAAAATAGCTGTAGAACTAAGCAATCGGATCGTTACGACTAGCATCGATG

| | |
|---|---|
| SEQ ID No. 101 | > PB.97.126.A_44-H1 |

GGGAGAGGAGAGAACGTTCTCGTGAGATGACTATGTTAAGATGACGCTGTTGGGATCGTTACGACTAGCATCGATG

| | |
|---|---|
| SEQ ID No. 102 | > PB.97.126.A_44-A2 |

GGGAGAGGAGAGAACGTTCTCGGGANACAAGGAACNCAATATTTAGTGAACGGGATCGTTACGACTAGCATCGATG

| | |
|---|---|
| SEQ ID No. 103 | > PB.97.126.A_44-B2 |

GGGAGAGGAGAGAACGTTCTCGCCAAGGAACACAATAGTTAGGTGAGAATCGGGATCGTTACGACTAGCATCGATG

| | |
|---|---|
| SEQ ID No. 104 | > PB.97.126.A_44-C2 |

GGGAGAGGAGAGAACGTTCTCGGTACAAGGAACACAATAGTTAGTGCCGTGGGATCGTTACGACTAGCATCGATG

| | |
|---|---|
| SEQ ID No. 105 | > PB.97.126.A_44-D2 |

GGGAGAGGAGAGAACGTTCTCGATTCAACGGTCCAAAAAAGCTGTAGTACTTAGGATCGTTACGACTAGCATCGATG

| | |
|---|---|
| SEQ ID No. 106 | > PB.97.126.A_44-E2 |

GGGAGAGGAGAGAACGTTCTCGCAATGCAAGGAACACAATAGTTAGCAGCCGGGATCGTTACGACTAGCATCGATG

| | |
|---|---|
| SEQ ID No. 107 | > PB.97.126.A_44-F2 |

GGGAGAGGAGAGAACGTTCTCGAAAGGAGAAAGCTGAAGTACTTACTATGCGGGATCGTTACGACTAGCATCGATG

| | |
|---|---|
| SEQ ID No. 108 | > PB.97.126.A_44-G2 |

GGGAGAGGAGAGAACGTTCTCGCACAAGGAACACAATAGTTAGTGCAAGACGGGATCGTTACGACTAGCATCGATG

| | |
|---|---|
| SEQ ID No. 109 | > PB.97.126.A_44-A3 |

GGGAGAGGAGAGAACGTTCTCGCACAAGGAACTACGAGTTAGTGTGGGAGTGGGATCGTTACGACTAGCATCGATG

| | |
|---|---|
| SEQ ID No. 110 | > PB.97.126.A_44-B3 |

GGGAGAGGAGAGAACGTTCTCGCACAAGGAACACAATAGTTAGTGCAAGACGGGATCGTTACGACTAGCATCGATA

| | |
|---|---|
| SEQ ID No. 111 | > PB.97.126.A_44-C3 |

GGGAGAGGAGAGAACGTTCTCGGCGGGAAAATAGCTGTAGTACTAACCCACGGATCGTTACGACTAGCATCGATG

TABLE 7

Corresponding cDNAs of the Thrombin Aptamer Sequences-2′-OH AG,
2′-OMe CU (rRmY)

| | |
|---|---|
| SEQ ID No. 112 | > PB.97.126.B_44-E3 |

GGGAGAGGAGAGAACGTTCTCGGCCTCAAGGAAAAGAAAATTTAGAGGCCCGGGATCGTTACGACTAGCATCGATG

| | |
|---|---|
| SEQ ID No. 113 | > PB.97.126.B_44-F3 |

GGGAGAGGAGAGAACGTTCTCGGAACAAGATAGCTGAAGGACTAAGTTTACGGGATCGTTACGACTAGCATCGATG

| | |
|---|---|
| SEQ ID No. 114 | > PB.97.126.B_44-G3 |

GGGAGAGGAGAGAACGTTCTCGGAACAAGATAGCTGAAGGACTAAGTTTACGGGATCGTTACGACTAGCATCGATG

| | |
|---|---|
| SEQ ID No. 115 | > PB.97.126.B_44-H3 |

GGGAGAGGAGAGAACGTTCTCGGAGCCAAGGAAACGAAGATTTAGGCTCATTGGGATCGTTACGACTAGCATCGATG

| | |
|---|---|
| SEQ ID No. 116 | > PB.97.126.B_44-A4 |

GGGAGAGGAGAGAACGTTCTCGATCACAAGAAATGTGGGANGGTAGTGATNCNNNTCGTTNCGACTAGCATCGATG

| | |
|---|---|
| SEQ ID No. 117 | > PB.97.126.B_44-B4 |

GGGAGAGGAGAGAACGTTCTCGTCGAAAGGGAGCTTTGTCTCGGGACAGAACGGATCGTTACGACTAGCATCGATG

| | |
|---|---|
| SEQ ID No. 118 | > PB.97.126.B_44-C4 |

GGGAGAGGAGAGAACGNTCTCGTGCAAAGATAGCTGGAGGACTAATGCGGCGGGATCGTTACGACTAGCATCGATG

| | |
|---|---|
| SEQ ID No. 119 | > PB.97.126.B_44-D4 |

GGGAGAGGAGAGAACGTTCTCGTCGAAAGGGAGCTTTGTCTCGGGACAGAACGGATCGTTACGACTAGCATCGATG

| | |
|---|---|
| SEQ ID No. 120 | > PB.97.126.B_44-E4 |

GGGAGAGGAGAGAACGTTCTCGNCNAAGGNGAGCTTTGTCCCNGGACANAANGNATCGTTACAACTAGCATCGATG

| | |
|---|---|
| SEQ ID No. 121 | > PB.97.126.B_44-F4 |

GGGAGAGGAGAGAACGTTCTCGGAACAAGATAGCTGAAGGACTAAGTTTACGGGATCGTTACGACTAGCATCGATG

| | |
|---|---|
| SEQ ID No. 122 | > PB.97.126.B_44-G4 |

GGGAGAGGAGAGAACGTTCTCGGAACAAGATAGCTGAAGGACTAAGTTTACGGGATCGTTACGACTAGCATCGATG

| | |
|---|---|
| SEQ ID No. 123 | > PB.97.126.B_44-H4 |

GGGAGAGGAGAGAACGTTCTCGGCGCAAAAAAAGCTGGAGTACTTAGTGTCGAGGGATCGTTACGACTAGCATCGATG

| | |
|---|---|
| SEQ ID No. 124 | > PB.97.126.B_44-A5 |

GGGAGAGGAGAGAACGTTCTCGTCGAAAGGGAGCTTTGTCTCGGGACAGAACGGATCGTTACGACTAGCATCGATG

| | |
|---|---|
| SEQ ID No. 125 | > PB.97.126.B_44-B5 |

GGGAGAGGAGAGAACGTTCTCGACACAAGAAAGCTGCAGAACTTAGGGTCGTGGATCGTTACGACTAGCATCGATG

| | |
|---|---|
| SEQ ID No. 126 | > PB.97.126.B_44-C5 |

GGGAGAGGAGAGAACGTTCTCGGAACNGGATTGTTGAAGGACTAANTTTACGGGATCGTTACGACTAGCATCGATG

| | |
|---|---|
| SEQ ID No. 127 | > PB.97.126.B_44-D5 |

GGGAGAGGAGAGAACGTTCTCGGCCTCAAGGGAAAGAAAATTTAGAGGCCCGGGATCGTTACGACTAGCATCGATG

| | |
|---|---|
| SEQ ID No. 128 | > PB.97.126.B_44-E5 |

GGGAGAGGAGAGAACGTTCTCGGAAACAAGCTTAGAAATTCGCACCCTTGCCGGGATCGTTACGACTAGCATCGATG

| | |
|---|---|
| SEQ ID No. 129 | > PB.97.126.B_44-F5 |

GGGAGAGGAGAGAACGTTCTCGAAAGAAAAAAGCTGGAGAACTTACTTCCGGGATCGTTACGACTAGCATCGATG

| | |
|---|---|
| SEQ ID No. 130 | > PB.97.126.B_44-G5 |

GGGAGAGGAGAGAACGTTCTCGGTGATTGTACTCACATAGAAATGGCAACACTGGGATCGTTACGACTAGCATCGATG

TABLE 8

Corresponding cDNAs of the Thrombin Aptamer Sequences-2′-OH G,
2′-OMe CUA (rGmH)

| | |
|---|---|
| SEQ ID No. 131 | > PB.97.126.C_44-H5 |

GGAGAGGAGAGAACGTTCTCGGGTTCAAGGAACATGATAGTTAGAACCCGCGGATCGTTACGACTAGCATCGATG

| | |
|---|---|
| SEQ ID No. 132 | > PB.97.126.C_44-A6 |

GGGAGAGGAGAGAACGTTCTCGTTCCGAAAGGAACACAATAGTTATCGGATTGGGATCGTTACGACTAGCATCGATG

| | |
|---|---|
| SEQ ID No. 133 | > PB.97.126.C_44-B6 |

GGGAGAGGAGAGAACGTTCTCGTCTGCAAGGAACACAATAGTTAGCATTGCGGGATCGTTACGACTAGCATCGATG

| | |
|---|---|
| SEQ ID No. 134 | > PB.97.126.C_44-C6 |

GGGAGAGGAGAGAACGTTCTCGGTACAAGGAACACAATAGTTAGTGCCGGGGATCGTTACGACTAGCATCGATG

| | |
|---|---|
| SEQ ID No. 135 | > PB.97.126.C_44-D6 |

GGGAGAGGAGAGAACGTTCTCGGAACTCAGAGATCCTATGTGGACCAGAGAGGATCGTTACGACTAGCATCGATG

| | |
|---|---|
| SEQ ID No. 136 | > PB.97.126.C_44-E6 |

GGGAGAGGAGAGAACGTTCTCGCTGAGCAAGGAACGTAATAGTTAGCCTGCGGGATCGTTACGACTAGCATCGATG

| | |
|---|---|
| SEQ ID No. 137 | > PB.97.126.C_44-F6 |

GGGAGAGGAGAGAACGTTCTCGNANNNATAAATGATGGATCNCTTATTGTNNAGGATCGTTACGACTAGCATCGATG

| | |
|---|---|
| SEQ ID No. 138 | > PB.97.126.C_44-G6 |

GGGAGAGGAGAGAACGTTCTCGGCTTGGAAAAATAGCTTTTGGGCATCCGGGATCGTTACGACTAGCATCGATG

| | |
|---|---|
| SEQ ID No. 139 | > PB.97.126.C_44-H6 |

GGGAGAGGAGAGAACGTTCTCGGGTTCAAGGAACATGATAGCTAGAACCCGCGGATCGTTACGACTAGCATCGATG

| | |
|---|---|
| SEQ ID No. 140 | > PB.97.126.C_44-A7 |

GGGAGAGGAGAGAACGTTCTCGGGTTCAAGGAACATGATAGTTAGAACCCGCGGATCGTTACGACTAGCATCGATG

| | |
|---|---|
| SEQ ID No. 141 | > PB.97.126.C_44-B7 |

GGGAGAGGAGAGAACGTTCTCGTGGGCAGGGAACACAATAGTTAGCCTACGCGGATCGTTACGACTAGCATCGATG

| | |
|---|---|
| SEQ ID No. 142 | > PB.97.126.C_44-C7 |

GGGAGAGGAGAGAACGTTCTCGCGTGAAAGGAACACAATAGTTATCGTGCGGGATCGTTACGACTAGCATCGATG

| | |
|---|---|
| SEQ ID No. 143 | > PB.97.126.C_44-D7 |

GGGAGAGGAGAGAACGTTCTCGCGAGGTTTATCCTAGACGACTAACCGCCTGGGGATCGTTACGACTAGCATCGATG

| | |
|---|---|
| SEQ ID No. 144 | > PB.97.126.C_44-F7 |

GGGAGAGGAGAGAACGTTCTCGTCTGCTAGGAACACAATAGTTAGCATTGCGGGATCGTTACGACTAGCATCGATG

| | |
|---|---|
| SEQ ID No. 145 | > PB.97.126.C_44-G7 |

GGGAGAGGAGAGAACGTTCTCGCACAAGGAACTACGAGTTAGTGTGGGAGTGGGGATCGTTACGACTAGCATCGATG

| | |
|---|---|
| SEQ ID No. 146 | > PB.97.126.C_44-H7 |

GGGAGAGGAGAGAACGTTCTCGTGACACGAGGAACTTAGAGTTAGTAGCACGAGGATCGTTACGACTAGCATCGATG

| | |
|---|---|
| SEQ ID No. 147 | > PB.97.126.C_44-A8 |

GGGAGAGGAGAGAACGTTCTCGGCGGCGAAGGAACACAATAGTTACGTCCCGGGATCGTTACGACTAGCATCGATG

| | |
|---|---|
| SEQ ID No. 148 | > PB.97.126.C_44-B8 |

GGGAGAGGAGAGAACGTTCTCGAGCCCAAAAAAGCTGAAGTACTTTGGGCAGGGATCGTTACGACTAGCATCGATG

TABLE 9

Corresponding cDNAs of the Thrombin Aptamer Sequences-2′-OMe AUGC (r/mGmH,
each G has a 90% probability of having a 2′-OMe group incorporated therein)

| | |
|---|---|
| SEQ ID No. 149 | > PB.97.126.D_44-D8 |

GGGAGAGGAGAGAACGTTCTCGGTACAAGGAACACAATAGTTAGTGCCGTGGGATCGTTACGACTAGCATCGATG

| | |
|---|---|
| SEQ ID No. 150 | > PB.97.126.D_44-E8 |

GGGAGAGGAGAGAACGTTCTCGGATCGTTACGACTAGCATCGATG

| | |
|---|---|
| SEQ ID No. 151 | > PB.97.126.D_44-G8 |

GGGAGAGGAGAGAACGTTCTCGTGCGCAAGGAACACAATAGTTAGGGCGCGAGGATCGTTACGACTAGCATTGATG

| | |
|---|---|
| SEQ ID No. 152 | > PB.97.126.D_44-H8 |

GGGAGAGGAGAGAACGTTCTCGGAATGGAAGGAACACAATAGTTACCAGACGGGATCGTTACGACTAGCATCGATG

| | |
|---|---|
| SEQ ID No. 153 | > PB.97.126.D_44-A9 |

GGGAGAGGAGAGAACGTTCTCGTCTGCAAGGAACACAATAGTTAGCATTGCGGGATCGTTACGACTAGCATCGATG

| | |
|---|---|
| SEQ ID No. 154 | > PB.97.126.D_44-B9 |

GGGAGAGGAGAGAACGTTCTCGAGACAAGACAGCTGGAGGACTAAGTCACGAGGATCGTTACGACTAGCATCGATG

| | |
|---|---|
| SEQ ID No. 155 | > PB.97.126.D_44-C9 |

GGGAGAGGAGAGAACGTTCTCGATGCCCGCAAAGGAACACGATAGTTATGCGGGATCGTTACGACTAGCATCGATG

| | |
|---|---|
| SEQ ID No. 156 | > PB.97.126.D_44-D9 |

GGGAGAGGAGAGAACGTTCTCGTCTGNNAGGAACACAATATTTAGCATTGCGGGATCGTTACGACTAGCATCGATG

| | |
|---|---|
| SEQ ID No. 157 | > PB.97.126.D_44-E9 |

GGGAGAGGAGAGAACGTTCTCGAATGTGCGGAGCAGTATTGGTACACTTTCGGGATCGTTACGACTAGCATCGATG

| | |
|---|---|
| SEQ ID No. 158 | > PB.97.126.D_44-F9 |

GGGAGAGGAGAGAACGTTCTCGCCAAGGAACACAATAGTTAGGTGAGAATCGGGATCGTTACGACTAGCATCGATG

| | |
|---|---|
| SEQ ID No. 159 | > PB.97.126.D_44-G9 |

GGGAGAGGAGAGAACGTTCTCGCCAAGGAACACAATAGTTAGGTGAGAATCGGGATCGTTACGACTAGCATCGATG

| | |
|---|---|
| SEQ ID No. 160 | > PB.97.126.D_44-H9 |

GGGAGAGGAGAGAACGTTCTCGGGAAGCAAGGAACTTAGAGTTAGTTGACCGGGATCGTTACGACTAGCATCGATG

| | |
|---|---|
| SEQ ID No. 161 | > PB.97.126.D_44-A10 |

GGGAGAGGAGAGAACGTTCTCGTGGGCAAGGAACACAATAGTTAGCCTACGCGGATCGTTACGACTAGCATCGATG

| | |
|---|---|
| SEQ ID No. 162 | > PB.97.126.D_44-B10 |

GGGAGAGGAGAGAACGTTCTCGTCGGGCATGGAACACAATAGTTAGACCGCGGGATCGTTACGACTAGCATCGATG

| | |
|---|---|
| SEQ ID No. 163 | > PB.97.126.D_44-C10 |

GGGAGAGGAGAGAACGTTCTCGGTCGCAAGGAACATAATAGTTAGCGGAGGGGATCGTTACGACTAGCATCGATG

| | |
|---|---|
| SEQ ID No. 164 | > PB.97.126.D_44-D10 |

GGGAGAGGAGAGAACGTTCTCGTCTGCAAGGAACACAATAGTTAGCATTGCGGGATCGTTACGACTAGCATCGATG

| | |
|---|---|
| SEQ ID No. 165 | > PB.97.126.D_44-E10 |

GGGAGAGGAGAGAACGTTCTCGCCGACAATCAGCTCGGATCGTGTGCTACGCTGGATCGTTACGACTAGCATCGATG

TABLE 10

Corresponding cDNAs of the Thrombin Aptamer Sequences-alternately “r/mGmH”
and 2′-OMe AUC, 2′-F G (toggle).

| | |
|---|---|
| SEQ ID No. 166 | > PB.97.126.E_44-F10 |

GGGAGAGGAGAGAACGTTCTCGAGACAAGATAGCTGAAGGACTAAGTCACGAGGGATCGTTACGACTAGCATCGATG

| | |
|---|---|
| SEQ ID No. 167 | > PB.97.126.E_44-G10 |

GGGAGAGGAGAGAACGTTCTCGGAACAAGATAGCTGAAGGACTAAGTTTGCGGGATCGTTACGACTAGCATCGATG

| | |
|---|---|
| SEQ ID No. 168 | > PB.97.126.E_44-H10 |

GGGAGAGGAGAGAACGTTCTCGGAGNCAAGGAAACNAATATTTAGGCTCANTGGNNNCNTTNCANCTAGCNNCNNTA

| | |
|---|---|
| SEQ ID No. 169 | > PB.97.126.E_44-A11 |

GGGAGAGGAGAGAACGTTCTCGTCTGCAAGGAACACAATAGTTAGCATTGCGGGATCGTTACGACTAGCATCGATG

| | |
|---|---|
| SEQ ID No. 170 | > PB.97.126.E_44-B11 |

GGGAGAGGAGAGAACGTTCTCGGAACAAGATAGCTGAAGGACTAAGTTTACGGGATCGTTACGACTAGCATCGATG

| | |
|---|---|
| SEQ ID No. 171 | > PB.97.126.E_44-C11 |

GGGAGAGGAGAGAACGTTCTCGGATCGTTACGACTAGCATCGATG

| | |
|---|---|
| SEQ ID No. 172 | > PB.97.126.E_44-D11 |

GGGAGAGGAGAGAACGTTCTCGGTGATAGTACTCACATAGAATGGCTACACTGGGATCGTTACGACTAGCATCGATG

| | |
|---|---|
| SEQ ID No. 173 | > PB.97.126.E_44-E11 |

GGGAGAGGAGAGAACGTTCTCGCCTGGGCAAGGAACAGAAAAGTTAGCGCCAGGATCGTTACGACTAGCATCGATG

| | |
|---|---|
| SEQ ID No. 174 | > PB.97.126.E_44-F11 |

GGGAGAGGAGAGAACGTTCTCGTAACGGACAAAAGGAACCGGGAAGTTATCTGGATCGTTACGACTAGCATCGATG

| | |
|---|---|
| SEQ ID No. 175 | > PB.97.126.E_44-G11 |

GGGAGAGGAGAGAACGTTCTCGCGCACAAGATAGAGAAGACTAAGTCCGCGGGGATCGTTACGACTAGCATCGATG

| | |
|---|---|
| SEQ ID No. 176 | > PB.97.126.E_44-H11 |

GGGAGAGGAGAGAACGTTCTCGCGCACAAGATAGAGAAGACTAAGTTCGCGGGGATCGTTACGACTAGCATCGATG

| | |
|---|---|
| SEQ ID No. 177 | > PB.97.126.E_44-A12 |

GGGAGAGGAGAGAACGTTCTCGCGCCAATAAAGCTGGAGTACTTAGAGCGCGGGATCGTTACGACTAGCATCGATG

| | |
|---|---|
| SEQ ID No. 178 | > PB.97.126.E_44-B12 |

GGGAGAGGAGAGAACGTTCTCGGGAAACAAGGAACTTAGAGTTAGTTGACCGGGATCGTTACGACTAGCATCGATG

| | |
|---|---|
| SEQ ID No. 179 | > PB.97.126.E_44-C12 |

GGGAGAGGAGAGAACGTTCTCGCTAGCAAGATAGGTGGGACTAAGCTAGTGAGGATCGTTACGACTAGCATCGATG

| | |
|---|---|
| SEQ ID No. 180 | > PB.97.126.E_44-D12 |

GGGAGAGGAGAGAACGTTCTCGTCGAAGGGGAGCTTTGTCTCGGGACAGAACGGATCGTTACGACTAGCATCGATG

| | |
|---|---|
| SEQ ID No. 181 | > PB.97.126.E_44-E12 |

GGGAGAGGAGAGAACGTTCTCGGAACAAGATAGCTGAAGGACTAAGTTTACGGGATCGTTACGACTAGCATCGATG

| | |
|---|---|
| SEQ ID No. 182 | > PB.97.126.E_44-G12 |

GGGAGAGGAGAGAACGTTCTCGGAACAAGATAGCTGAAGGACTAAGTTTGCGGGATCGTTACGACTAGCATCGATG

| | |
|---|---|
| SEQ ID No. 183 | > PB.97.126.E_44-H12 |

GGGAGAGGAGANNTCCCCNCNCGGAAAAANAAAAAAGAAGAANTANGTTNGGGGGATCGTTACGACTAGCATCGATG

TABLE 11

Stabilized Aptamer Sequences (each G residue has 90% probability of being
substituted with a 2′-OMe group, “3T” refers to an inverted thymidine
nucleotide attached to the phosphodiester backbone at the 5′ position,
the resulting oligo having two 5′-OH ends and is thus resistant to 3′
nucleases).

SEQ ID No. 184 ARC224-Stabilized VEGF Aptamer
5′ mCmGmAmUmAmUmGmCmAmGmUmUmUmGmAmGmAmAmGmUmCmGmCmGmCmAmUmUmCmG-3 T
SEQ ID No. 185 ARC225-Stabilized VEGF Aptamer
5′ mCmGmAmUmAmUGmCmAGmUmUmUGmAGmAmAGmUmCGmCGmCmAmUmUmCmG-3T
SEQ ID No. 186 ARC226 Single-hydroxy VEGF aptamer
5′ mGmAmUmCmAmUmGmCmAmUGmUmGmGmAmUmCmGmCmGmGmAmUmC-3T
SEQ ID No. 187 ARC245 VEGF Aptamer
5′ mAmUmGmCmAmGmUmUmUmGmAmGmAmAmGmUmCmGmCmGmCmAmU-3T
SEQ ID No, 188 ARC259 hVEGF Aptamer-C-G base pair swap of ARC245 (2nd base
pair in) which has improved binding over ARC245.
5′ mAmCmGmCmAmGmUmUmUmGmAmGmAmAmGmUmCmGmCmGmCmGmU-3′

### Example 2

### 2′-OMe SELEX™

Libraries of transcription templates were used to generate pools of RNA oligonucleotides incorporating 2′-O-methyl NTPs under various transcription conditions. The transcription template (ARC256) and the transcription conditions are described below as rRmY (SEQ ID NO:456), rGmH (SEQ ID NO:462), r/mGmH (SEQ ID NO:463), and dRmY (SEQ ID NO:464). The unmodified RNA transcript is represented by SEQ ID NO:468.

ARC256: DNA Transcription Template

| | |
|---|---|
| 5′-CATCGATCGATCGATCGACAGCGNNNNNNNNNNNNNNNNNNNNNNNNNN | (SEQ ID NO:453) |
| NNNNGTAGAACGTTCTCTCCTCTCCCTATAGTGAGTCGTATTA-3′ | |

The ARC256 RNA Transcription Product is:

| | |
|---|---|
| 5′-GGGAGAGGAGAGAACGUUCUACNNNNNNNNNNNNNNNNNNNNNNNNNN | (SEQ ID NO:468) |
| NNNNCGCUGUCGAUCGAUCGAUCGAUG-3′ | |

The transcription conditions were varied as follows where 1× Tc buffer is 200 mM HEPES, 40 mM DTT, 2 mM Spermidine, 0.01% Triton X-100, pH 7.5.

When 2′-OMe C and U and 2′-OH A and G (rRmY) conditions were used, the transcription reaction conditions were 1× Tc buffer, 50-200 nM double stranded template (200 nm template was used for round 1, and for subsequent rounds approximately 50 nM, a 1/10 dilution of an optimized PCR reaction, using conditions described herein, was used), 9.6 mM MgCl₂, 2.9 mM MnCl₂, 2 mM each base, 10% PEG-8000, 0.25 units inorganic pyrophosphatase, and 1.5 units Y639F/H784A T7 RNA polymerase. One unit of the Y639F/H784A mutant T7 RNA polymerase is defined as the amount of enzyme required to incorporate 1 nmole of 2′-OMe NTPs into transcripts under the r/mGmH conditions. One unit of inorganic pyrophosphatase is defined as the amount of enzyme that will liberate 1.0 mole of inorganic orthophosphate per minute at pH 7.2 and 25° C.

When 2′-OMe A, C, and U and 2′-OH G (rGmH) conditions were used, the transcription reaction conditions were 1× Tc buffer, 50-200 nM double stranded DNA template (200 nm template was used for round 1, and for subsequent rounds approximately 50 nM, a 1/10 dilution of an optimized PCR reaction, using conditions described herein was used), 9.6 mM MgCl₂, 2.9 mM MnCl₂, 2 mM each base, 10% PEG-8000, 0.25 units inorganic pyrophosphatase, and 1.5 units Y639F single mutant T7 RNA polymerase. One unit of the Y639F mutant T7 RNA polymerase is defined as the amount of enzyme required to incorporate 1 nmole of 2′-OMe NTPs into transcripts under the r/mGmH conditions.

When all 2′-OMe nucleotides (r/mGmH) conditions were used, the reaction conditions were 1× Tc buffer, 50-200 nM double stranded template (200 nm template was used for round 1, and for subsequent rounds approximately 50 nM, a 1/10 dilution of an optimized PCR reaction, using conditions described herein was used), 6.5 mM MgCl₂, 2 mM MnCl₂, 1 mM each base, 30 μM GTP, 1 mM GMP, 10% PEG-8000, 0.25 units inorganic pyrophosphatase, and 1.5 units Y639F/H784A T7 RNA polymerase.

When deoxy purines, A and G, and 2′-OMe pyrimidines (dRmY) conditions were used, the reaction conditions were 1× Tc buffer, 50-300 nM double stranded template (300 nm template was used for round 1, and for subsequent rounds approximately 50 nM, a 1/10 dilution of an optimized PCR reaction, using conditions described herein was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

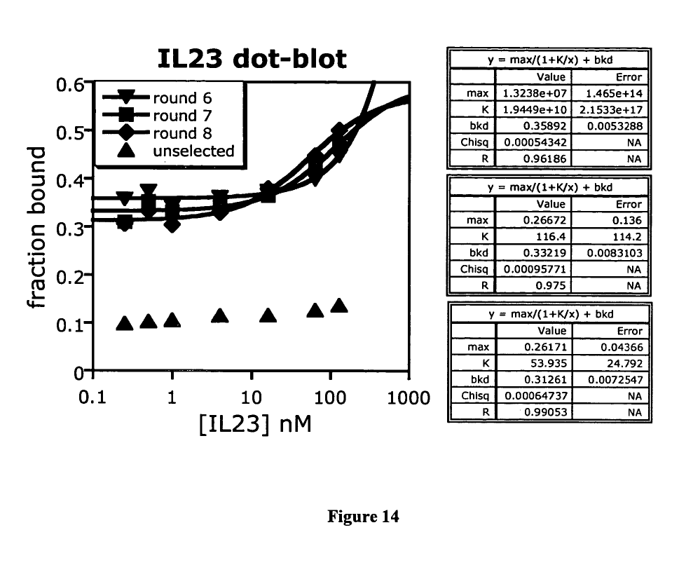

These pools were then used in SELEX™ to select for aptamers against the following targets: IgE, IL-23, PDGF-BB, thrombin and VEGF. A plot of dRmY Round 6, 7, 8, and unselected sequences binding to target IL-23 is shown in FIG. 14, and a plot of dRmY Round 6, 7, and unselected sequences binding to target PDGF-BB is shown in FIG. 14.

### Example 3

### dRmY SELEX™ of Aptamers Against IgE

While fully 2′-OMe substituted oligonucleotides are the most stable modified aptamers, substituting the purines with deoxy purine nucleotides also results in stable transcripts. When dRmY (deoxy purines, A and G, and 2′-OMe pyrimidines) transcription conditions are used, the products are very DNase-resistant and useful as stable therapeutics. This result is surprising since the composition of the dRmY transcripts is approximately 50% DNA, which is notoriously easily degraded by nucleases. Also, when dRmY transcription conditions are used, there is no requirement for a 2′-OH GTP spike. Studies have shown that approximately the same amount of dRmY transcripts having modified nucleotides are produced with 2′-OH GTP doping as without 2′-OH GTP doping. Accordingly, under dRmY transcription conditions, 2′-OH GTP doping is optional. Libraries of transcription templates were used to generate pools of oligonucleotides incorporating 2′-O-methylpyrimidine NTPs (U and C) and deoxy purines (A and G) NTPs under various transcription conditions. The transcription template (ARC256) and the transcription conditions are described below as dRmY.

ARC256: DNA Transcription Template

| | |
|---|---|
| 5′-CATCGATCGATCGATCGACAGCGNNNNNNNNNNNNNNNNNNNNNNNNNN | (SEQ ID NO:453) |
| NNNNGTAGAACGTTCTCTCCTCTCCCTATAGTGAGTCGTATTA-3′ | |

The ARC256 dRmY RNA Transcription Product is:

| | |
|---|---|
| 5′-GGGAGAGGAGAGAACGCGUUCUACNNNNNNNNNNNNNNNNNNNNNNNNNN | (SEQ ID NO:464) |
| NNNNCGCUGUCGAUCGAUCGAUCGAUG-3′ | |

When deoxy purines, A and G, and 2′-OMe pyrimidines (dRmY) conditions were used, the reaction conditions were 1× Tc buffer, 50-300 nM double stranded template (300 nm template was used for round 1, and for subsequent rounds approximately 50 nM, a 1/10 dilution of an optimized PCR reaction, using conditions described herein, was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

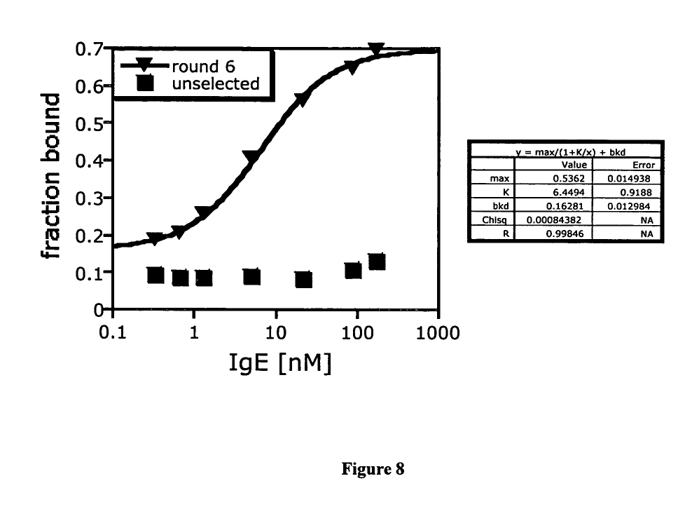

These pools were then used in SELEX™ to select for aptamers against IgE as a target. The sequences obtained after round 6 of SELEX™ as described above are listed in Table 12 below. A plot of Round 6 sequences bound with increasing target IgE concentration is shown in FIG. 8.

Table 12-Corresponding cDNAs of the Round 6 sequences of dRmY SELEX™ against IgE.

| | | |
|---|---|---|
| SEQ ID No. 190 | IgE A5 | GGGAGAGGAGAGAACGTTCTACAGGCAGTTCTGGGGACCCATGGGGGAAGTGCGCTGTCGATCGATCGATCGATG |
| SEQ ID No. 191 | IgE A6 | GGGAGAGGAGAGAACGTTCTACGATTAGCAGGGAGGAGTGCGAAGAGGACGCTGTCGATCGATCGATCGATG |
| SEQ ID No. 192 | IgE A7 | GGGAGAGGAGGACGTTCTACACTCTGGGGACCCGTGGGGGAGTGCAGCAACGCTGTCGATCGATCGATCGATG |
| SEQ ID No. 193 | IgE A8 | GGGAGAGGAGAGAACGTTCTACAAGCAGTTCTGGGGACCCATGGGGGAAGTGCGCTGTCGATCGATCGATCGATG |
| SEQ ID No. 194 | IgE B5 | GGGAGAGGAGAGAACGTTCTACGAGGTGAGGGTCTACAATGGAGGGATGGTCGCTGTCGATCGATCGATCGATG |
| SEQ ID No. 195 | IgE B6 | GGGAGAGGAGAGAACGTTCTACCCGCAGCATAGCCTGNGGACCCATGNGGGGCGCTGTCGATCGATCGATCGATG |
| SEQ ID No. 196 | IgE B7 | GGGAGAGGAGAGAACGTTCTACTGGGGGGCGTGTTCATTAGCAGCGTCGTGTCGCTGTCGATCGATCGATCGATG |
| SEQ ID No. 197 | IgE B8 | GGGAGAGGAGAGAACGTTCTACAGGCAGTTCTGGGGACCCATGGGGGAAGTGCGCTGTCGATCGATCGATCGATG |
| SEQ ID No. 198 | IgE C5 | GGGAGAGGAGAGAACGTTCTACGCAGCGCATCTGGGGACCCAAGAGGGGATTCGCTGTCGATCGATCGATCGATG |
| SEQ ID No. 199 | IgE C6 | GGGAGAGGAGAGAACGTTCTACAGGCAGTTCTGGGGACCCATGGGGGAAGTGCGCTGTCGATCGATCGATCGATG |
| SEQ ID No. 200 | IgE C7 | GGGAGAGGAGAGAACGTTCTACGGGATGGGTAGTTGGATGGAAATGGGAACGCTGTCGATCGATCGATCGATG |
| SEQ ID No. 201 | IgE C8 | GGGAGAGGAGAGAACGTTCTACGAGGTGTAGGGATAGAGGGGTGTAGGTAACGCTGTCGATCGATCGATCGATG |
| SEQ ID No. 202 | IgE D5 | GGGAGAGGAGAGAACGTTCTACAGGAGTGGAGCTACAGAGAGGGTTAGGGGTCGCTGTCGATCGATCGATCGATG |
| SEQ ID No. 203 | IgE D6 | GGGAGAGGAGAGAACGTTCTACGGATGTTGGGAGTGATAGAAGGAAGGGGAGCGCTGTCGATCGATCGATCGATG |
| SEQ ID No. 204 | IgE D7 | GGGAGAGGAGAGAACGTTCTACAGGCAGTTCTGGGGACCCATGGGGGAAGTGCGCTGTCGATCGATCGATCGATG |
| SEQ ID No. 205 | IgE D8 | GGGAGAGGAGAGAACGTTCTACAGGCAGTTCTGGGGACCCATGGGGGAAGTGCGCTGTCGATCGATCGATCGATG |
| SEQ ID No. 206 | IgE E5 | GGGAGAGGAGAGAACGTTCTACAGGCAGTTCTGGGGACCCATGGGGGAAGTGCGCTGTCGATCGATCGATCGATG |
| SEQ ID No. 207 | IgE E6 | GGGAGAGGAGAGAACGTTCTACTTGGGGTGGAAGGAGTAAGGGAGGTGCTGATCGCTGTCGATCGATCGATCGATG |
| SEQ ID No. 208 | IgE E7 | GGGAGAGGAGAGAACGTTCTACGTATTAGGGGGGAAGGGGACGAATAGATCACGCTGTCGATCGATCGATCGATG |
| SEQ ID No. 209 | IgE E8 | GGGAGAGGAGAGAACGTTCTACAGGGAGAGAGTGTTGAGTGAAGAGGAGGAGTCGCTGTCGATCGATCGATCGATG |
| SEQ ID No. 210 | IgE F5 | GGGAGAGAGAGAACGTTCTACATTGTGCTCCTGGGGCCCAGTGGGGAGCCACGCTGTCGATCGATCGATCGATG |
| SEQ ID No. 211 | IgE F6 | GGGAGAGGAGAGAACGTTCTACGAGCAGCCCTGGGGCCCGGAGGGGGATGGTCGCTGTCGATCGATCGATCGATG |
| SEQ ID No. 212 | IgE F7 | GGGAGAGGAGAGAACGTTCTACAGGCAGTTCTGGGGACCCATGGGGGAAGTGCGCTGTCGATCGATCGATCGATG |
| SEQ ID No. 213 | IgE F8 | GGGAGAGGAGAGAACGTTCTACCAACGGCATCCTGGGCCCCGGGGATGTCGCTGTCGATCGATCGATCGATG |
| SEQ ID No. 214 | IgE G5 | GGGAGAGGAGAGAACGTTCTACGAGTGGATAGGGAAGAAGGGGAGTAGTCACGCTGTCGATCGATCGATCGATG |
| SEQ ID No. 215 | IgE G6 | GGGAGAGGAGAGAACGTTCTACCCGCAGCATAGCCTGGGGACCCATGGGGCGCTGTCGATCGATCGATCGATG |
| SEQ ID No. 216 | IgE G7 | GGGAGAGGAGAGAACGTTCTACGGTCGCGTGTGGGGGACGGATGGGTATTGGTCGCTGTCNATCGATCGATCNATG |
| SEQ ID No. 217 | IgE G8 | GGGAGAGGAGAGAACGTTCTACCCGCAGCATAGCCTGGGGACCCATGGGGGGCGCTGTCGATCGATCGATCGATG |
| SEQ ID No. 218 | IgE H5 | GGGAGAGGAGAGAACGTTCTACCCGCAGCATAGCCTGGGGACCCATGGGGGGCGCTGTCGATCGATCGATCGATG |
| SEQ ID No. 219 | IgE H6 | GGGAGAGGAGAGAACGTTCTACGGGGTTACGTCGCACGATACATGCATTCATCGCTGTCGATCGATCGATCGATG |
| SEQ ID No. 220 | IgE H7 | GGGAGAGGAGAGAACGTTCTACTAGCGAGGAGGGGTTTTCTATTTTTGCGATCGCTGTCGATCGATCGATCGATG |

### Example 4

### dRmY SELEX™ of Aptamers against Thrombin

While fully 2′-OMe substituted oligonucleotides are the most stable modified aptamers, substituting the purines with deoxy purine nucleotides also results in stable transcripts. When dRmY (deoxy purines, A and G, and 2′-OMe pyrimidines) transcription conditions are used, the products are very DNase-resistant and useful as stable therapeutics. This result is surprising since the composition of the dRmY transcripts is approximately 50% DNA, which is notoriously easily degraded by nucleases. Also, when dRmY transcription conditions are used, there is no requirement for a 2′-OH GTP spike. Libraries of transcription templates were used to generate pools of oligonucleotides incorporating 2′-O-methylpyrimidine NTPs (U and C) and deoxy purines (A and G) NTPs under various transcription conditions. The transcription template (ARC256) and the transcription conditions are described below as dRmY.

ARC256: DNA Transcription Template

| | |
|---|---|
| 5′-dCATCGATCGATCGATCGACAGCGNNNNNNNNNNNNNNNNNNNNNNNNN | (SEQ ID NO:453) |
| NNNNNGTAGAACGTTCTCTCCTCTCCCTATAGTGAGTCGTATTA-3′ | |

The ARC256 dRmY RNA transcription product is:

| | |
|---|---|
| 5′-GGGAGAGGAGAGAACGUUCUACNNNNNNNNNNNNNNNNNNNNNNNNNN | (SEQ ID NO:464) |
| NNNNCGCUGUCGAUCGAUCGAUCGAUG-3′ | |

When deoxy purines, A and G, and 2′-OMe pyrimidines (dRmY) conditions were used, the reaction conditions were 1× Tc buffer, 50-300 nM double stranded template (300 nm template was used for round 1, and for subsequent rounds a 1/10 dilution of an optimized PCR reaction, using conditions described herein, was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

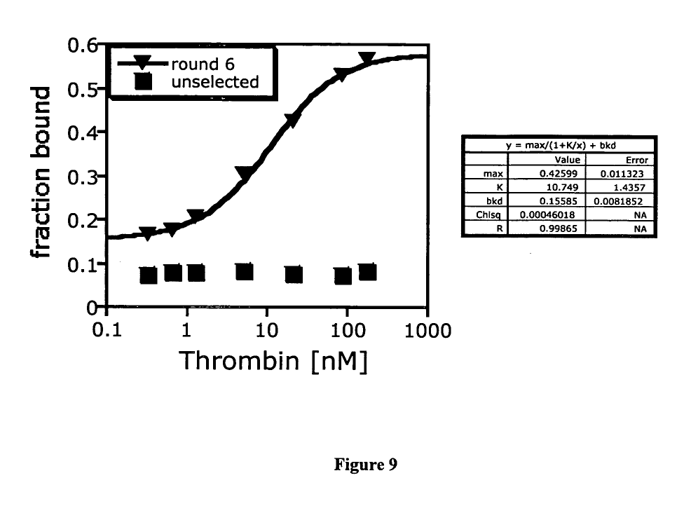

These pools were then used in SELEX™ to select for aptamers against thrombin as a target. The sequences obtained after round 6 of SELEX™ as described above are listed in Table 13 below. A plot of Round 6 sequences bound to target thrombin is shown in FIG. 9.

TABLE 13
Corresponding cDNAs of the Round 6 sequences of dRmY SELEX™ against thrombin.

| | |
|---|---|
| SEQ ID No. 221 | Thrombin A1 |

GGGAGAGGAGAGAACGTTCTACGTGTGATGGGTGAGAGGATGAGTTAGTGACGCTGTCGATCGATCGATCGATG

| | |
|---|---|
| SEQ ID No. 222 | Thrombin A2 |

GGGAGAGGAGAGAACGTTCTACAATGGGAGGGTAATAGTGATGAGGAGAGGCGCTGTCGATCGATCGATGATG

| | |
|---|---|
| SEQ ID No. 223 | Thrombin A3 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 224 | Thrombin A4 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 225 | Thrombin B1 |

GGGAGAGGAGAGAACGTTCTACAGGTAGCGTGAGGGGGTGTTAATAGAGGGGCGCTGTCGATCGATCGATCGATG

| | |
|---|---|
| SEQ ID No. 226 | Thrombin B2 |

GGGAGAGGAGAGAACGTTCTACGATAGGATGGGTGGGACAGGAGAGGGAGTGCGCTGTCGATCGATCGATCGATG

| | |
|---|---|
| SEQ ID No. 227 | Thrombin B3 |

GGGAGAGGAGAGAACGTTCTACCAGTGAGGGCAGTGTCAGATTGAGAGGAGGCGCTGTCGATCGATCGATCGATG

| | |
|---|---|
| SEQ ID No. 228 | Thrombin B4 |

GGGAGAGGAGAGAACGTTCTACCTTGCCTAACAGGAGGTGGAGTATTGGACCCGCTGTCGATCGATCGATCGATG

| | |
|---|---|
| SEQ ID No. 229 | Thrombin C1 |

GGGAGAGGAGAGAACGTTCTACCTTGCCTAACAGGAGGTGGAGTATTGGACCCGCTGTCGATCGATCGATCGATG

| | |
|---|---|
| SEQ ID No. 230 | Thrombin C2 |

GGGAGAGGAGAGAACGTTCTACGTCGTGAGTAATGGCTCGTAGATGAGGTCGCTGTCGATCGATCGATCGATG

| | |
|---|---|
| SEQ ID No. 231 | Thrombin C3 |

GGGAGAGGAGAGAACGTTCTACGGGATTAAGAGGGGAGAGGAGCAGTTGAGCGCTGTCGATCGATCGATCGATG

| | |
|---|---|
| SEQ ID No. 232 | Thrombin C4 |

GGGAGAGGAGAGAACGTTCTACTCCGGTTGGGGTATCAGGTCTACGGACTGACGCTGTCGATCGATCGATCGATG

| | |
|---|---|
| SEQ ID No. 233 | Thrombin D1 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 234 | Thrombin D2 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 235 | Thrombin D3 |

GGGAGAGGAGAGAACGTTCTACATGACAAGAGGGGGTTGTGTGGGATGGCAGCGCTGTCGATCGATCGATCGATG

| | |
|---|---|
| SEQ ID No. 236 | Thrombin D4 |

GGGAGAGGAGAGAACGTTCTACACAGGGAGGGGAGCGGAGAGGAGAGAGGGTACGCTGTCGATCGATCGATCGATG

| | |
|---|---|
| SEQ ID No. 237 | Thrombin E1 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 238 | Thrombin E2 |

GGGAGAGGAGAGAACGTTCTACGTCGTGAGTAATGGCTCGTAGATGAGGTCGCTGTCGATCGATCGATCGATG

| | |
|---|---|
| SEQ ID No. 239 | Thrombin E4 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 240 | Thrombin F1 |

GGGAGAGGAGAGAACGTTCTACGGGTCGTGAGATAATGGCTCCCGTATTCAGCGCTGTCGATCGATCGATCCGTG

| | |
|---|---|
| SEQ ID No. 241 | Thrombin F2 |

GGGAGAGGAGAGAACGTTCTACCTTGCTAACAGGAGGTGGAGTATTGGACCCGCTGTCGATCGATCGATCGATG

| | |
|---|---|
| SEQ ID No. 242 | Thrombin F3 |

GGGAGAGGAGAGAACGTTCTACGGCTATGCGTCGTGAGTCAATGGCCCGCATCGCTGTCGATCGATCGATCGATG
SEQ ID No. 243 Thrombin F4
GGGAGAGGAGAGAACGTTCTACGGGTCGTGAGATAGTGGCTCCCGTATTCAGCGCTGTCGATCGATCGATCGATG

| | |
|---|---|
| SEQ ID No. 244 | Thrombin G1 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 245 | Thrombin G2 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 246 | Thrombin G3 |

GGGAGAGGAGAGAACGTTCTACCTTGTCTAACAGGAGGTGGAGTATTGGACCCGCTGTCGATCGATCGATCGATG

| | |
|---|---|
| SEQ ID No. 247 | Thrombin G4 |

GGGAGAGGAGAGAACGTTCTACGACTTTGAGGGTGGTGAGAGTGGAAGAGAGCGCTGTCGATCGATCGATCGATG

| | |
|---|---|
| SEQ ID No. 248 | Thrombin H1 |

GGGAGAGGAGAGAACGTTCTACGGTAGGGTATGACCAGGGAGGTATTGGAGGCGCTGTCGATCGATCGATCGATG

| | |
|---|---|
| SEQ ID No. 249 | Thrombin H2 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 250 | Thrombin H3 |

GGGAGAGGAGAGAACGTTCTACGGGTCGTGAGATAATGGCTCCCGTATTCAGCGCTGTCGATCGATCGATCGATG

| | |
|---|---|
| SEQ ID No. 251 | Thrombin H4 |

GGGAGAGGAGAGAACGTTCTACGTTATGCATGTGGAGAGTGAGAGAGGGCGCTGTCGATCGATCGATCGATG

### Example 5

### dRmY SELEX™ of Aptamers against VEGF

While fully 2′-OMe substituted oligonucleotides are the most stable modified aptamers, substituting the purines with deoxy purine nucleotides also results in stable transcripts. When dRmY (deoxy purines, A and G, and 2′-OMe pyrimidines) transcription conditions are used, the products are very DNase-resistant and useful as stable therapeutics. This result is surprising since the composition of the dRmY transcripts is approximately 50% DNA RNA, which is notoriously easily degraded by nucleases. Also, when dRmY transcription conditions are used, there is no requirement for a 2′-OH GTP spike. Libraries of transcription templates were used to generate pools of oligonucleotides incorporating 2′-O-methylpyrimidine NTPs (U and C) and deoxy purines (A and G) NTPs under various transcription conditions. The transcription template (ARC256) and the transcription conditions are described below as dRmY.

ARC256: DNA Transcription Template

| | |
|---|---|
| 5′-CATCGATCGATCGATCGACAGCGNNNNNNNNNNNNNNNNNNNNNNNNNN | (SEQ ID NO:453) |
| NNNNGTAGAACGTTCTCTCCTCTCCCTATAGTGAGTCGTATTA-3′ | |

ARC256 dRmY Transcription Product is:

| | |
|---|---|
| 5′-GGGAGAGGAGAGAACGUUCUACNNNNNNNNNNNNNNNNNNNNNNNNNN | (SEQ ID NO:464) |
| NNNNCGCUGUCGAUCGAUCGAUCGAUG-3′ | |

When deoxy purines, A and G, and 2′-OMe pyrimidines (dRmY) conditions were used, the reaction conditions were 1× Tc buffer, 50-300 nM double stranded template (300 nm template was used for round 1, and for subsequent rounds a 1/10 dilution of an optimized PCR reaction, using conditions described herein, was used), 9.6 mM MgCl₂, 2.9 mM MnCl₂, 2 mM each base, 30 μM GTP, 2 mM Spermine, 10% PEG-8000, 0.25 units inorganic pyrophosphatase, and 1.5 units Y639F single mutant RNA polymerase.

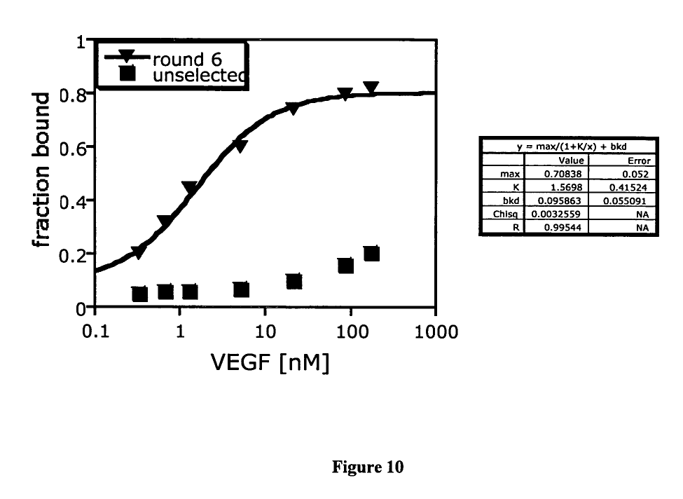

These pools were then used in SELEX™ to select for aptamers against VEGF as a target. The sequences obtained after round 6 of SELEX™ as described above are listed in an alignment show in Table 14 below. A plot of Round 6 sequences bound to target VEGF is shown in FIG. 10.

Table 14—Corresponding cDNAs of the Round 6 sequences of dRmY SELEX™ against VEGF.

TABLE 13
Corresponding cDNAs of the Round 6 sequences of dRmY SELEX™ against VEGF.

| | |
|---|---|
| SEQ ID No. 252 | VEGF A9 |

GGGAGAGGAGAGAACGTTCTACCATGTCTGCGGGAGGTGAGTAGTGATCCTGCGCTGTCGATCGATCGATCGATG

| | |
|---|---|
| SEQ ID No. 253 | VEGF A10 |

GGGAGAGGAGAGAACGTTCTACAGAGTGGGAGGGATGTGTGACACAGGTAGGCGCTGTCGATCGATCGATCGATG

| | |
|---|---|
| SEQ ID No. 254 | VEGF A11 |

GGGAGAGGAGAGAACGTTCTACGCTCCATGACAGTGAGGTGAGTAGTGATCGCTGTCGATCGATCGATCGATG

| | |
|---|---|
| SEQ ID No. 255 | VEGF A12 |

GGGAGAGGAGAGAACGTTCT CGATGCTGACAGGGTGTGTTCAGTAATGGCTCGCTGTCGATCGATCGATCGATG

| | |
|---|---|
| SEQ ID No. 256 | VEGF B9 |

GGGAGAGGAGAGAACGTTCTACCAGCAAACAGGGTCAGGTGAGTAGTGATGACGCTGTCGATCGATCGATCGATG

| | |
|---|---|
| SEQ ID No. 257 | VEGF B10 |

GGGAGAGGAGAGAACGTTCTACGACAAGCCGGGGGTGTTCAGTAGTGGCAACCGCTGTCGATCGATCGATCGATG

| | |
|---|---|
| SEQ ID No. 258 | VEGF B11 |

GGGAGAGGAGAGAACGTTCTACATATGGCGCTGGAGGTGAGTAATGATCGTGCGCTGTCGATCGATCGATCGATG

| | |
|---|---|
| SEQ ID No. 259 | VEGF B12 |

GGGAGAGGAGAGAACGTTCTACGGGGCGATAGCGTTCAGTAGTGGCGCCGGTCGCTGTCGATCGATCGATCGATG

| | |
|---|---|
| SEQ ID No. 260 | VEGF C9 |

GGGAGAGGAGAGAACGTTCTACATAGCGGACTGGGTGCATGGAGCGGCGCACGGCTGTCGATCGATCGATCGATG

| | |
|---|---|
| SEQ ID No. 261 | VEGF C10 |

GGGAGAGGAGAGAACGTTCTACGGGTCAACAGGGGCGTTCAGTAGTGGCGGCGCTGTCGATCGATCGATCGATG

| | |
|---|---|
| SEQ ID No. 262 | VEGF C11 |

GGGAGAGGAGAGAACGTTCTACGCATGCGAGCTGAGGTGAGTAGTGATCAGTCGCTGTCGATCGATCGATCGATG

| | |
|---|---|
| SEQ ID No. 263 | VEGF C12 |

GGGAGAGGAGAGAACGTTCTACATGCGACAGGGGAGTGTTCAGTAGTGGCACGCTGTCGATCGATCGATCGATG

| | |
|---|---|
| SEQ ID No. 264 | VEGF D9 |

GGGAGAGGAGAGAACGTTCTACCCCATCGTATGGAGTGCGGAACGGGGCATACGCTGTCGATCGATCGATCGATG

| | |
|---|---|
| SEQ ID No. 265 | VEGF D10 |

GGGAGAGGAGAGAACGTTCTACAGTGAGGCGGGAGCGTTTCAGTAATGGCGCTGTCGATCGATCGATCGATG

| | |
|---|---|
| SEQ ID No. 266 | VEGF D12 |

GGGAGAGGAGAGAACGTTCTACACAGCGTCGGGTGTTCAGTAATGGCGCAGCGCTGTCGATCGATCGATCGATG

| | |
|---|---|
| SEQ ID No. 267 | VEGF E9 |

GGGAGAGGAGAGAACGTTCTACGGTGTTCAGTAGTGGCACAGGAGGAAGGGATGCTGTCGATCGATCGATCGATG

| | |
|---|---|
| SEQ ID No. 268 | VEGF E10 |

GGGAGAGGAGAGAACGTTCTACAGTTCAGGCGTTAGGCATGGGTGTCGCTTTCGCTGTCGATCGATCGATCGATG

| | |
|---|---|
| SEQ ID No. 269 | VEGF E11 |

GGGAGAGGAGAGAACGTTCTACATGCGACATGCGAGTGTTCAGTAGCGGCAGCGCTGTCGATCGATCGATCGATG

| | |
|---|---|
| SEQ ID No. 270 | VEGF E12 |

GGGAGAGGAGAGAACGTTCTACCTATGGCGTTACAGCGAGGTGAGTAGTGATCGCTGTCGATCGATCGATCGATG

| | |
|---|---|
| SEQ ID No. 271 | VEGF F9 |

GGGAGAGGAGAGAACGTTCTACCAGCCGATCCAGCCAGGCGTTCAGTAGTGGCGCTGTCGATCGATCGATCGATG

| | |
|---|---|
| SEQ ID No. 272 | VEGF F10 |

GGGAGAGGAGAGAACGTTCTACGGCACAGGCACGGCGAGGTGAGTAATGATCGCTGTCGATCGATCGATCGATG

| | |
|---|---|
| SEQ ID No. 273 | VEGF G9 |

GGGAGAGGAGAGAACGTTCTACTGTGGACAGCGGGAGTGCGGAACGGGGTCGCTGTCGATCGATCGATCGATG

| | |
|---|---|
| SEQ ID No. 274 | VEGF G10 |

GGGAGAGGAGAGAACGTTCTACTGATGCTGCGAGTGCATGGGGCAGGCGCTTCGCTGTCGATCGATCGATCGATG

| | |
|---|---|
| SEQ ID No. 275 | VEGF G11 |

GGGAGAGGAGAGAACGTTCTACGGTACAATGGGAATGACAGTGATGGGTAGCCGCTGTCGATCGATCGATCGATG

| | |
|---|---|
| SEQ ID No. 276 | VEGF G12 |

GGGAGAGGAGAGAACGTTCTACATGGACAGCGAAGCATGGGGGAGGCGCACGCTGTCGATCGATCGATCGATG

| | |
|---|---|
| SEQ ID No. 277 | VEGF H9 |

GGGAGAGGAGAGAACGTTCTACTGGGAGCGACAGTGAGCATGGGGTAGGCGCCGCTGTCGATCGATCGATCGATG

| | |
|---|---|
| SEQ ID No. 278 | VEGF H11 |

GGGAGAGGAGAGAACGTTCTACCGGCGAGCAGGTGTTCAGTAGTGGCTTTGCGCTGTCGATCGATCGATCGATG

| | |
|---|---|
| SEQ ID No. 279 | VEGF H12 |

GGGAGAGGAGAGAACGTTCTACGATCAGTGAGGGAGTGCAGTAGTGGCTCGTCGCTGTCGATCGATCGATCGATG

### Example 6

### Plasma Stability of 2′-OMe NTPs (mN) and dRmY oligonucleotides

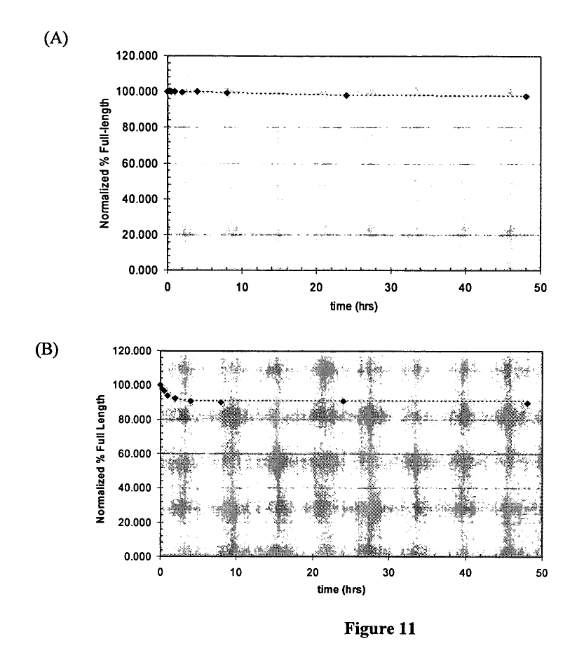

An oligonucleotide of two sequences linked by a polyethylene glycol polymer (PEG) was synthesized in two versions: (1) with all 2′-OMe NTPs (mN): 5′-GGAGCAGCACC-3′ (SEQ ID NO:457)-[PEG]-GGUGCCAAGUCGUUGCUCC-3′ (SEQ ID NO:458) and (2) with 2′-OH purine NTPs and 2′-OMe pyrimidines (dRmY) GGAGCAGCACC-3′ (SEQ ID NO:465)-[PEG]-GGUGCCAAGUCGUUGCUCC-3′ (SEQ ID NO:466). These oligonucleotides were evaluated for full length stability. FIG. 11A shows a degradation plot of the all 2′-OMe oligonucleotide with 3′idT and FIG. 11B shows a degradation plot of the dRmY oligonucleotide. The oligonucleotides were incubated at 50 nM in 95% rat plasma at 37° C. and show a plasma half-life of much greater than 48 hours for each, and that they have very similar plasma stability profiles.

### Example 7

### rRmY and rGmH 2′-OMe SELEX™ Against Human IL-23

Selections were performed to identify aptamers containing 2′-OMe C, U and 2′-OH G, A (rRmY), and 2′-O-Methyl A, C, and U and 2′-OH G (rGmH). All selections were direct selections against human IL-23 protein target which had been immobilized on a hydrophobic plate. Selections yielded pools significantly enriched for h-IL-23 binding versus naïve, unselected pool. Individual clone sequences for h-IL-23 are reported herein, but h-IL-23 binding data for the individual clones are not shown.

Pool Preparation. A DNA template with the sequence 5′-GGGAGAGGAGAGAACGTTCTACNNNNNNNNNNNNNNNNNNNNNNNNNNNNNNCG CTGTCGATCGATCGATCGATG-3′ (SEQ ID NO:459) was synthesized using an ABI EXPEDITE™ DNA synthesizer, and deprotected by standard methods. The templates were amplified with the primers PB.118.95.G: 5′-GGGAGAGGAGAGAACGTTCTAC-3′ (SEQ ID NO:460) and STC.104.102.A (5′-CATCGATCGATCGATCGACAGC-3′ (SEQ ID NO:461) and then used as a template (200 nm template was used for round 1, and for subsequent rounds approximately 50 nM, a 1/10 dilution of an optimized PCR reaction, using conditions described herein, was used) for in vitro transcription with Y639F single mutant T7 RNA polymerase. Transcriptions were done using 200 mM HEPES, 40 mM DTT, 2 mM spermidine, 0.01% TritonX-100, 10% PEG-8000, 5 mM MgCl₂, 1.5 mM MnCl₂, 500 μM NTPs, 500 μM GMP, 0.01 units/μl inorganic pyrophosphatase, and Y639F single mutant T7 polymerase. Two different compositions were transcribed rRmY and rGmH.

Selection. Each round of selection was initiated by immobilizing 20 pmoles of h-IL-23 to the surface Nunc Maxisorp hydrophobic plates for 2 hours at room temperature in 100 μL of 1× Dulbecco's PBS. The supernatant was then removed and the wells were washed 4 times with 120 μL wash buffer (1×DPBS, 0.2% BSA, and 0.05% Tween-20). Pool RNA was heated to 90° C. for 3 minutes and cooled to room temperature for 10 minutes to refold. In round 1, a positive selection step was conducted. Briefly, 1×10¹⁴ molecules (0.2 nmoles) of pool RNA were incubated in 100 μL binding buffer (1× DPBS and 0.05% Tween-20) in the wells with immobilized protein target for 1 hour. The supernatant was then removed and the wells were washed 4× with 120 μL wash buffer. In subsequent rounds a negative selection step was included. The pool RNA was also incubated for 30 minutes at room temperature in empty wells to remove any plastic binding sequences from the pool before the positive selection step. The number of washes was increased after round 4 to increase stringency. In all cases, the pool RNA bound to immobilized h-IL-23 was reverse transcribed directly in the selection plate after by the addition of RT mix (3′ primer, STC.104.102.A, and Thermoscript RT, Invitrogen) followed by incubated at 65° C. for 1 hour. The resulting cDNA was used as a template for PCR (Taq polymerase. New England Biolabs) “Hot start” PCR conditions coupled with a 60° C. annealing temperature were used to minimize primer-dimer formation. Amplified pool template DNA was desalted with a Centrisep column according to the manufacturer's recommended conditions and used to program transcription of the pool RNA for the next round of selection. The transcribed pool was gel purified on a 10% polyacrylamide gel every round. Table 15 shows the RNA pool concentrations used per round of selection.

TABLE 15
RNA pool concentrations per round of selection.

| pmoles | rRmY | | | | rGmH | | | |
|---|---|---|---|---|---|---|---|---|
| Pool used | 3OMe | | | | 3OMe | | | |
| Round | IL23 | hIgE | mIgE | PDGF-BB | IL23 | hIgE | mIgE | PDGF-BB |
| 1 | 200 | 200 | 200 | 200 | 200 | 200 | 200 | 200 |
| 2 | 110 | 140 | 130 | 135 | 40 | 50 | 40 | 60 |
| 3 | 65 | 115 | 60 | 160 | 100 | 190 | 90 | 160 |
| 4 | 50 | 40 | 40 | 30 | 170 | 120 | 40 | 240 |
| 5 | 80 | 130 | 130 | 110 | 100 | 60 | 40 | 70 |
| 6 | 100 | 80 | 90 | 39 | 110 | 140 | 90 | 90 |
| 7 | 50 | 90 | 130 | 170 | 70 | 80 | 130 | 90 |
| 8 | 120 | | 190 | 150 | 60 | 90 | 110 | 130 |
| 9 | 120 | | 210 | 170 | 80 | 80 | 100 | 100 |
| 10 | 130 | | 210 | 180 | | | | |
| 11 | 110 | | | 210 | | | | |

The selection progress was monitored using a sandwich filter binding assay. The 5′³²P-labeled pool RNA was refolded at 90° C. for 3 minutes and cooled to room temperature for 10 minutes. Next, pool RNA (trace concentration) was incubated with h-IL-23 DPBS plus 0.1 mg/ml tRNA for 30 minutes at room temperature and then applied to a nitrocellulose and nylon filter sandwich in a dot blot apparatus (Schleicher and Schuell). The percentage of pool RNA bound to the nitrocellulose was calculated and monitored approximately every 3 rounds with a single point screen (+/−250 nM h-IL-23). Pool K_{D} measurements were measured using a titration of protein and the dot blot apparatus as described above.

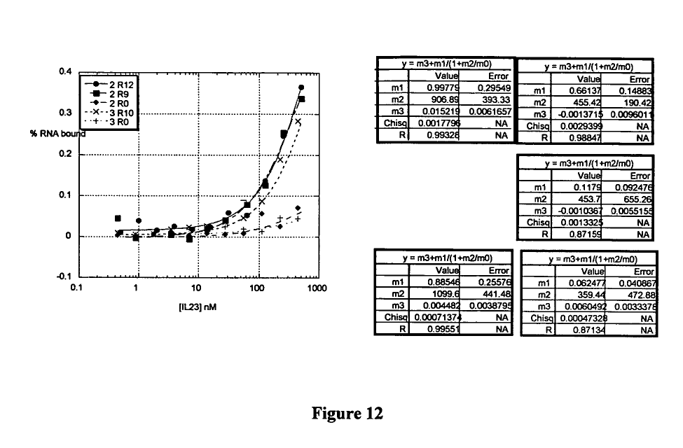

Selection. The rRmY h-IL-23 selection was enriched for h-IL-23 binding vs. the naïve pool after 4 rounds of selection. The selection stringency was increased and the selection was continued for 8 more rounds. At round 9 the pool K_{D} was approximately 500 nM or higher. The rGmH selection was enriched over the naïve pool binding at round 10. The pool KD is also approximately 500 nM or higher. The pools were cloned using TOPO TA cloning kit (Invitrogen) and individual sequences were generated. FIG. 12 shows pool binding data to h-IL-23 for the rGmH round 10 and rRmY round 12 pools. Dissociation constants were estimated fitting data to the equation: fraction RNA bound=amplitude*K_{D}/(K_{D}+[h-IL-23]). Table 16 shows the individual clone sequences for round 12 of the rRmY selection. There is one group of 6 duplicate sequences and 4 pairs of 2 duplicate sequences out of 48 clones. All 48 clones will be labeled and tested for binding to 200 mM h-IL-23. Table 17 shows the individual clone sequences for round 10 of the rGmH selection. Binding data is shown in FIG. 14.

TABLE 16

Corresponding cDNAs of the Individual Clone Sequences for Round 12 of
the rRmY Selection.

| | |
|---|---|
| SEQ ID No. 280 | ARX34P2.G01 |

GGGAGAGGAGAGAACGTTCTACAAATGAGAGCAGGCCGAAAAGGAGTCGCTCGCTGTCGATCGATCGATCGATGAAGGGCG

| | |
|---|---|
| SEQ ID No. 281 | ARX34P2.A06 |

GGGAGAGGAGAGAACGTTCTACAAAGGATCAATCTTTCGGCGTATGTGTGAGCGCTGTCGATCGATCGATCGATGAAGGGCG

| | |
|---|---|
| SEQ ID No. 282 | ARX34P2.E02 |

GGGAGAGGAGAGAACGTTCTACGGTAAGCAGGCTGACTGAAAAGGTTGAAGTCGCTGTCGATCGATCGATCGATGAAGGGCG

| | |
|---|---|
| SEQ ID No. 283 | ARX34P2.H05 |

GGGAGAGGAGAGAACGTTCTACAGGTTAAAAGCAGGCTCAGGAATGGAAGTCGCTGTCGATCGATCGATCGATGAAGGGCG

| | |
|---|---|
| SEQ ID No. 284 | ARX34P2.G04 |

GGGAGAGGAGAGAACGTTCTACAACAAAGCAGGCTCATAGTAATATGGAAGTCGCTGTCGATCGATCGATCGATGAAGGGCG

| | |
|---|---|
| SEQ ID No. 285 | ARX34P2.G03 |

GGGAGAGGAGAGAACGTTCTACAAAAGAGAGCAGGCCGAAAAGGAGTCGCTCGCTGTCGATCGATCGATCGATGAAGGGCG

| | |
|---|---|
| SEQ ID No. 286 | ARX34P2.H06 |

GGGAGAGGAGAGAACGTTCTACAAAAGGCAGGCTCAGGGGATCACTGGAAGTCGCTGTCGATCGATCGATCGATGAAGGGCG

| | |
|---|---|
| SEQ ID No. 287 | ARX34P2.B01 |

GGGAGAGGAGAGAACGTTCTACAAAAAGCAGGCCGTATGGATATAAGGGAGTCGCTGTCGATCGATCGATCGATGAAGGGCG

| | |
|---|---|
| SEQ ID No. 288 | ARX34P2.B03 |

GGGAGAGGAGAGAACGTTCTACAAAAGTGCAGGCTGCAGACATATGCGAAGTCGCTGTCGATCGATCGATCGATGAAGGGCG

| | |
|---|---|
| SEQ ID No. 289 | ARX34P2.D05 |

GGGAGAGGAGAGAACGTTCTACAAAGGAGAGCAGGCCGAAAAGGAGTCGCTCGCTGTCGATCGATCGATCGATGAAGGGCG

| | |
|---|---|
| SEQ ID No. 290 | ARX34P2.C05 |

GGGAGAGGAGAGAACGTTCTACAAGATATAATTAAGGATAAGTGCAGGAGACGCTGTCGATCGATCGATCGATGAAGGGCG

| | |
|---|---|
| SEQ ID No. 291 | ARX34P2.C04 |

GGGAGAGGAGAGAACGTTCTACAGACAACAGCNAGAGGGAATCNCANACAAAGACGCTGTCGATCGATCGATCGATGAAGGGCG

| | |
|---|---|
| SEQ ID No. 292 | ARX34P2.E06 |

GGGAGAGGAGAGAACGTTCTACAGATTCTAAGCGCAGGAATAAGTCACCAGACGCTGTCGATCGATCGATCGATGAAGGGCG

| | |
|---|---|
| SEQ ID No. 293 | ARX34P2.A01 |

GGGAGAGGAGAGAACGTTCTACGAATGAGCATGGAAGTGGGAGTACGTGCCGCTGTCGATCGATCGATCGATGAAGGGCG

| | |
|---|---|
| SEQ ID No. 294 | ARX34P2.C06 |

GGGAGAGGAGAGAACGTTCTACGAAAGAGGCGCCGGAAGTGAGAGTAAGTGCGCTGTCGATCGATCGATCGATGAAGGGCG

| | |
|---|---|
| SEQ ID No. 295 | ARX34P2.B04 |

GGGAGAGGAGAGAACGTTCTACGAAGTGAGTTTCCGAAGTGAGAGTACGAAACGCTGTCGATCGATCGATCGATGAAGGGCG

| | |
|---|---|
| SEQ ID No. 296 | ARX34P2.E04 |

GGGAGAGGAGAGAACGTTCTACGAATGAGAGCAGGCCGAAAAGGAGTCGCTCGCTGTCGATCGATCGATCGATGAAGGGCG

| | |
|---|---|
| SEQ ID No. 297 | ARX34P2.H04 |

GGGAGAGGAGAGAACGTTCTACGAGAGGCAAGAGAGAGTCGCATAAAAAAGACGCTGTCGATCGATCGATCGATGAAGGGCG

| | |
|---|---|
| SEQ ID No. 298 | ARX34P2.B06 |

GGGAGAGGAGAGAACGTTCTACGCAGGCTGTCGTAGACAAACGATGAAGTCGCGCTGTCGATCGATCGATCGATGAAGGGCG

| | |
|---|---|
| SEQ ID No. 299 | ARX34P2.F05 |

GGGAGAGGAGAGAACGTTCTACGGAAAAAGATATGAAAGAAAGGATTAAGAGACGCTGTCGATCGATCGATCGATGAAGGGCG

| | |
|---|---|
| SEQ ID No. 300 | ARX34P2.H02 |

GGGAGAGGAGAGAACGTTCTACGGAAGGNAACAANAGCACTGTTTGTGCAGGCGCTGTCGATCNATCNATCNATGAAGGGCG

| | |
|---|---|
| SEQ ID No. 301 | ARX34P2.C03 |

GGGAGAGGAGAGAACGTCTACGGAGCATANGGCNTGAAACTGAGANAGTAACGCTGTCGATCGATCGATCGATGAAGGGCG

| | |
|---|---|
| SEQ ID No. 302 | ARX34P2.D01 |

GGGAGAGGAGAGAACGTTCTACGAAAAAGGATATGAGAGAAAGGATTAAGAGACGCTGTCGATCGATCGATCGATGAAGGGCG

| | |
|---|---|
| SEQ ID No. 303 | ARX34P2.A03 |

GGGAGAGGAGAGAACGTTCTACATACATAGGCGCCGCGAATGGGAAAGAAAGCGCTGTCGATCGATCGATCGATGAAGGGCG

| | |
|---|---|
| SEQ ID No. 304 | ARX34P2.B02 |

GGGAGAGGAGAGAACGTTCTACTCATGAAGCCATGGTTGTAATTCTGTTTGGCGCTGTCGATCGATCGATCGATGAAGGGCG

| | |
|---|---|
| SEQ ID No. 305 | ARX34P2.C01 |

GGGAGAGGAGAGAACGTTCTACTAATGCAGGCTCAGTTACTACTGGAAGTCGCTGTCGATCGATCGATCGATGAAGGGCG

| | |
|---|---|
| SEQ ID No. 306 | ARX34P2.D06 |

GGGAGAGGAGAGAACGTTCTACTTTCATAGGCGGGATTATGGAGGAGTATTCGCTGTCGATCGATCGATCGATGAAGGGCG

| | |
|---|---|
| SEQ ID No. 307 | ARX34P2.G05 |

AGGAGAGGAGAGAACGTTCTACTAGAAGCAGGCTCGAATACAATTCGGAAGTCGCTGTCGATCGATCGATCGATGAAGGGCG

| | |
|---|---|
| SEQ ID No. 308 | ARX34P2.F06 |

GGGAGAGGAGAGAACGTTCTACTTAGCGATGTGGAAGAGAGAGTACGAGGACGCTGTCGATCGATCGATCGATGAAGGGCG

| | |
|---|---|
| SEQ ID No. 309 | ARX34P2.F02 |

GGGAGAGGAGAGAACGTTCTACTTGCGAAGACCGTGGAAGAGGAGTACTGGTCGCTGTCGATCGATCGATCGATGAAGGGCG

| | |
|---|---|
| SEQ ID No. 310 | ARX34P2.B05 |

GGGAGAGGAGAGAACGTTCTACTTTTGGTGAAGGTGTAAGAGTGGCACTACACGCTGTCGATCGATCGATCGATGAAGGGCG

| | |
|---|---|
| SEQ ID No. 311 | ARX34P2.A05 |

GGGAGAGGAGAGAACGTTCTACCATCAGTTGTGGCGATTATGTGGGAGTATGCGCTGTCGATCGATCGATCGATGAAGGGCG

| | |
|---|---|
| SEQ ID No. 312 | ARX34P2.E03 |

GGGAGAGGAGAGAACGTTCTACANAANAACATGCGATTAAAGATCATGAACAGCGCTGTCGATCGATCGATCGATGAAGGGCG

| | |
|---|---|
| SEQ ID No. 313 | ARX34P2.F04 |

GGGAGAGGAGAGAACGTTCTACATAAGCAGGCTCCGATAGTATTCGGGAAGTCGCTGTCGATCGATCGATCGATGAAGGGCG

TABLE 17

Corresponding cDNAs of the Individual Clone Sequences for Round 10 of
the rGmH Selection.

| | |
|---|---|
| SEQ ID No. 314 | ARX34P2.E10 |

GGGAGAGGAGAGAACGTTCTACTTTCGGAATGCGATGGGGGTGATTCGTGGTCGCTGTCGATCGATCGATCGATGAAGGGCG

| | |
|---|---|
| SEQ ID No. 315 | ARX34P2.H09 |

GGGAGAGGAGAGAACGTTCTACCTGTTGAGGCTAAGTGGATGATTGAGGGGCTGTCGATCGATCGATCGATGAAGGGCG

| | |
|---|---|
| SEQ ID No. 316 | ARX34P2.A07 |

GGGAGAGGAGAGAACGTTCTACCTGGGTCGGTGCGTTGGAGATGTCGTTGCGCTGTCGATCGATCGATCGATGAAGGGCG

| | |
|---|---|
| SEQ ID No. 317 | ARX34P2.A12 |

GGGAGAGGAGAGAACGTTCTACCTGATGTCGTTGTTTGGAGATTATCTGACNCTGTCNATCGATCGATCGATGAAGGGCG

| | |
|---|---|
| SEQ ID No. 318 | ARX34P2.A08 |

GGGAGAGGAGAGAACGTTCTACCTCGCGCGACGAGCGAATTTCCGGATGCGGCGCTGTCGATCGATCGATCGATGAAGGGCG

| | |
|---|---|
| SEQ ID No. 319 | ARX34P2.D12 |

GGGAGAGGAGAGAACGTTCTACCATGAATGATTGCGATCGTTGTTCGTGTGGCGCTGTCGATCGATCGATCGATGAAGGGCG

| | |
|---|---|
| SEQ ID No. 320 | ARX34P2.E11 |

GGGAGAGGAGAGACGTTCTACTCCGACCACGCCTGGGTGATTCCTACNACGACGCTGTCGATCGATCGATCGATGAAGGGCG

| | |
|---|---|
| SEQ ID No. 321 | ARX34P2.E12 |

GGGAGAGGAGAGAACGTTCTACTACTTTTGGGGATTCACTCCGCGCTGATGTCGCTGTCGATCGATCGATCGATGAAGGGCG

| | |
|---|---|
| SEQ ID No. 322 | ARX34P2.D08 |

GGGAGAGGAGANAACGTTCTANTAGTGCTTGCGAGATAGTGTAGGATTATACTGCTGTCGATCGATCGATCGATGAAGGGCG

| | |
|---|---|
| SEQ ID No. 323 | ARX34P2.F07 |

GGGAGAGGAGAGAACGTTCTACTAGTGTCCTTCTCCACGTGGTTGTAATTGTCGCTGTCGATCGATCGATCGATGAAGGGCG

| | |
|---|---|
| SEQ ID No. 324 | ARX34P2.B11 |

GGGAGAGGAGAGAACGTTCTACTATTGTGGCGCTTGTTGGACTAACTGACTACGCTGTCGATCGATCGATCGATGAAGGGCG

| | |
|---|---|
| SEQ ID No. 325 | ARX34P2.F12 |

GGGAGAGGAGGACGTCCTACTTCGATTGTGATCTTGTGCGGCCTGTGAGCGCTGTCGATCGATCGATCGATCGATGAAGGGCG

| | |
|---|---|
| SEQ ID No. 326 | ARX34P2.A09 |

GGGAGAGGAGAGAACGTTCTACTTGGCGATGTCGGAAGAGAGAGTCACGAGGGCGCTGTCGATCGATCGATCGATGAAGGGCG

| | |
|---|---|
| SEQ ID No. 467 | ARX34P2.B07 |

GGGAGAGGAGAGAACGTTCTACTTGCTGTGACGGACGGGCTTGAGAGGCTCGCTGTCGATCGATCGATCGATGAAGGGCG

| | |
|---|---|
| SEQ ID No. 327 | ARX34P2.D07 |

GGGAGAGGAGAGAACGTTCTACTTGAANCTGCGTGAATTGANAGTAACGAAGCGCTGTCAATCGATCNATCAATNAAGGGCG

| | |
|---|---|
| SEQ ID No. 328 | ARX34P2.H10 |

GGGAGAGGAGAGAACGTTCTACTCGAGAGGACATGTGGATCCGGTTCGCGTGCGCTGTCGATCGATCGATCGATGAAGGGCG

| | |
|---|---|
| SEQ ID No. 329 | ARX34P2.H07 |

GGGAGAGGAGAGAACGTTCTACTGTGATGCGGTTTGCGTCGACCGGATTCGTCGCTGTCGATCGATCGATCGATGAAGGGCG

| | |
|---|---|
| SEQ ID No. 330 | ARX34P2.F11 |

GGGAGAGGAGAGAACGTTCTACTGTGTGATTGGGCGCATGTCGAGGCGACACGCTGTCGATCGATCGATCGATGAAGGGCG

| | |
|---|---|
| SEQ ID No. 331 | ARX34P2.G07 |

GGGAGAGGAGAGAACGTTCTACTGATTAAGATGCGCTGGTAGAGCGGTGGGCGCTGTCGATCGATCGATCGATGAAGGGCG

| | |
|---|---|
| SEQ ID No. 332 | ARX34P2.A10 |

GGGAGAGGAGAGAACGTTCTACTGGTTAATTTGCATGCGCGANTAACNTGNTCGCTGTCGATCGATCGATCGATGAAGGGCG

| | |
|---|---|
| SEQ ID No. 333 | ARX34P2.G10 |

GGGAGAGGAGAGAACGTTCTACTGGGAAGCGGTAACTTGGATTGACCGATCCGCTGTCGATCGATCGATCGATGAAGGGCG

| | |
|---|---|
| SEQ ID No. 334 | ARX34P2.H11 |

GGGAGAGGAGAGAACGTTCTACTGTTACGGAGATGATGGGTTTGGCTGTTGGCGCTGTCGATCGATCGATCGATGAAGGGCG

| | |
|---|---|
| SEQ ID No. 335 | ARX34P2.C07 |

GGGAGAGGAGAGAACGTTCTACTTGTGGACTGAGATACGATTCGGAGCTGGCGCTGTCGATCGATCGATCGATGAAGGGCG

| | |
|---|---|
| SEQ ID No. 336 | ARX34P2.E08 |

GGGAGAGGAGAGAACGTTCTACTTGTGAGTTTCCTTGGGCCTTGAGCGTGGGCGCTGTCGATCGATCGATCGATGAAGGGCG

| | |
|---|---|
| SEQ ID No. 337 | ARX34P2.A11 |

GGGAGAGGAGAGAACGTTCTACAGGTGATGTGAGCCGATTGTGAAGTTTTGTCGCTGTCGATCGATCGATCGATGAAGGGCG

| | |
|---|---|
| SEQ ID No. 338 | ARX34P2.B08 |

GGGAGAGGAGAGAACGTTCTACAGCGGATGTTTGGGGGTGTGTGTTGGTGTCGCTGTCGATCGATCGATCGATGAAGGGCG

| | |
|---|---|
| SEQ ID No. 339 | ARX34P2.B09 |

GGGAGAGGAGAGAACGTTCTACATGCGGTGGTGGTCTTTCGATGGGTGGAAGTCGCTGTCGATCGATCGATCGATGAAGGGCG

| | |
|---|---|
| SEQ ID No. 340 | ARX34P2.B12 |

GGGAGAGGAGAGAACGTTCTACATTGGAGGGGCGCATGTGGTCTGTTTATGCGCTGTCGATCGATCGATCGATGAAGGGCG

| | |
|---|---|
| SEQ ID No. 341 | ARX34P2.F10 |

GGGAGAGGAGAGAACGTTCTACGTGTTTCGCGGATTTGAAGAGGAGTAAAATCGCTGTCGATCGATCGATCGATGAAGGGCG

| | |
|---|---|
| SEQ ID No. 342 | ARX34P2.B10 |

GGGAGAGGAGAGAACGTTCTACGTGTGCGTGTTCGGGAAGGGAGAGTGCCGAGGCTGTCGATCGATCGATCGATGAAGGGCG

| | |
|---|---|
| SEQ ID No. 343 | ARX34P2.G08 |

GGGAGAGGAGAGAACGTTCTACGTGTGTGGTGTGCGATGCTTGGCTGTTTGTCGCTGTCGATCGATCGATCGATGAAGGGCG

| | |
|---|---|
| SEQ ID No. 344 | ARX34P2.C08 |

GGGAGAGGAGAGAACGTTCTACGGTTTGTGTGGCTTGGATCTGAAGACTAAGCGCTGTCGATCGATCGATCGATGAAGGGCG

| | |
|---|---|
| SEQ ID No. 345 | ARX34P2.F09 |

GGGAGAGGAGAGAACGTTCTACGGTTCTGGGCTTGTGTGTGAGGATTGACGGCGCTGTCGATCGATCGATCGATGAAGGGCG

| | |
|---|---|
| SEQ ID No. 346 | ARX34P2.C10 |

GGGAGAGGAGAGAACGTTCTACGATGATGAAGGCGAAAAGACGAGGCTGTCGATCGATCGATCGATGAAGGGCG

| | |
|---|---|
| SEQ ID No. 347 | ARX34P2.C11 |

GGGAGAGGAGAGAACGTTCTACGAGTGCTGATGCGTGTCCTGGGATGGAATTCGCTGTCGATCGATCGATCGATGAAGGGCG

| | |
|---|---|
| SEQ ID No. 348 | ARX34P2.D09 |

GGGAGAGGAGAGAACGTTCTACGCGTTTATAGCGATCGATGATGATATAGGCCGCTGTCGATCGATCGATCGATGAAGGGCG

| | |
|---|---|
| SEQ ID No. 349 | ARX34P2.D10 |

GGGAGAGGAGAGAACGTTCTACGCGTTCAAATGGGATAGAATTGGCTGCGGGCGCTGTCGATCGATCGATCGATGAAGGGCG

| | |
|---|---|
| SEQ ID No. 350 | ARX34P2.D11 |

GGGAGAGGAGAGAACGTTCTACGAAATTGTGCGTCAGTGTGAGGCGGTTTGCTGTCGATCGATCGATCGATGAAGGGCG

| | |
|---|---|
| SEQ ID No. 351 | ARX34P2.E07 |

GGGAGAGGAGAGAACGTTCTACGGTCGAAATGAGGGTCTGGAGTTCCGACGACGCTGTCGATCGATCGATCGATGAAGGGCGG

| | |
|---|---|
| SEQ ID No. 352 | ARX34P2.E09 |

GGGAGAGGAGAGAACGTTCTACGAATTTGGTAATCTGGGTGACTTAGGATGTCGCTGTCGATCGATCGATCGATGAAGGGCG

| | |
|---|---|
| SEQ ID No. 353 | ARX34P2.G12 |

GGGAGAGGAGAGAACGTTCTACGATTTTTTGTGCCGAAGTAAGAGTACGCGCGCTGTCGATCGATCGATCGATGAAGGGCG

| | |
|---|---|
| SEQ ID No. 354 | ARX34P2.H08 |

AGGAGAGGAGAGAACGTTCTACGGAGTGTGCGCGGATGAAAACAGAAGTTGTCGCTGTCNATCGATCNATCAATGAAGGGCG

### Example 8

### rRmY 2′-OMe SELEX™ Against Human IgE

Selections were performed to identify aptamers containing 2′-OMe C, U and 2′-OH G, A (rRmY). All selections were direct selections against human IgE protein target which had been immobilized on a hydrophobic plate. Selections yielded pools significantly enriched for h-IgE binding versus naïve, unselected pool. Individual clone sequences for h-IgE are reported herein, but h-IgE binding data for the individual clones are not shown.

Pool Preparation. A DNA template with the sequence 5′-GGGAGAGGAGAGAACGTTCTACNNNNNNNNNNNNNNNNNNNNNNNNNNNNNNCG CTGTCGATCGATCGATCGATG-3′ (SEQ ID NO:459) was synthesized using an ABI EXPEDITE™ DNA synthesizer, and deprotected by standard methods. The templates were amplified with the primers PB. 118.95.G 5′-GGGAGAGGAGAGAACGTTCTAC-3′(SEQ ID NO:460) and STC.104.102.A 5′-CATCGATCGATCGATCGACAGC-3′(SEQ ID NO:461) and then used as a template (200 nm template was used for round 1, and for subsequent rounds approximately 50 nM, a 1/10 dilution of an optimized PCR reaction, using conditions described herein, was used) for in vitro transcription with Y639F single mutant T7 RNA polymerase. Transcriptions were done using 200 mM HEPES, 40 mM DTT, 2 mM spermidine, 0.01% TritonX-100, 10% PEG-8000, 5 mM MgCl₂, 1.5 mM MnCl₂, 500 μM NTPs, 500 μM GMP, 0.01 units/μl inorganic pyrophosphatase, and Y639F single mutant T7 polymerase.

Selection. Each round of selection was initiated by immobilizing 20 pmoles of h-IgE to the surface Nunc Maxisorp hydrophobic plates for 2 hours at room temperature in 100 μL of 1× Dulbecco's PBS. The supernatant was then removed and the wells were washed 4 times with 120 μL wash buffer (1× DPBS, 0.2% BSA, and 0.05% Tween-20). Pool RNA was heated to 90° C. for 3 minutes and cooled to room temperature for 10 minutes to refold. In round 1, a positive selection step was conducted. Briefly, 1×10¹⁴ molecules (0.2 nmoles) of pool RNA were incubated in 100 μL binding buffer (1× DPBS and 0.05% Tween-20) in the wells with immobilized protein target for 1 hour. The supernatant was then removed and the wells were washed 4× with 120 μL wash buffer. In subsequent rounds a negative selection step was included. The pool RNA was also incubated for 30 minutes at room temperature in empty wells to remove any plastic binding sequences from the pool before the positive selection step. The number of washes was increased after round 4 to increase stringency. In all cases, the pool RNA bound to immobilized h-IgE was reverse transcribed directly in the selection plate after by the addition of RT mix (3′ primer, STC. 104.102.A, and Thermoscript RT, Invitrogen) followed by incubated at 65° C. for 1 hour. The resulting cDNA was used as a template for PCR (Taq polymerase, New England Biolabs) “Hot start” PCR conditions coupled with a 60° C. annealing temperature were used to minimize primer-dimer formation. Amplified pool template DNA was desalted with a Centrisep column according to the manufacturer's recommended conditions and used to program transcription of the pool RNA for the next round of selection. The transcribed pool was gel purified on a 10% polyacrylamide gel every round.

rRmY pool selection against h-IgE was enriched after 4 rounds over the naïve pool. The selection stringency was increased and the selection was continued for 2 more rounds. At round 6 the pool K_{D} is approximately 500 nM or higher. The pools were cloned using TOPO TA cloning kit (Invitrogen) and submitted for sequencing. The pool contained one dominant clone (AMX(123).A1)—which made up 71% of the clones sequenced. Three additional clones were tested and showed a higher extent of binding than the dominant clone. The K_{D}s for the pools were calculated to be approximately 500 nM. The dissociations constants were also calculated as described above. Table 18 shows the rRmY pool clones after Round 6 of selection to h-IgE where the dominant clone was AMX(123).A1 making up 40% of the 96 clones, along with 8 other sequence families.

TABLE 18

Corresponding cDNAs of the Individual Clone Sequence of rRmY Pool Clones
After Round 6 of Selection to h-IgE.

| | |
|---|---|
| SEQ ID No. 355 | AMX(123).A1 |

GGGAGAGGAGAGAACGTTCTACGATCTGGGCGAGCCAGTCTGACTGAGGAAGCGCTGTCGATCGATCGATCGATGAAGGGCG

| | |
|---|---|
| SEQ ID No. 356 | ARX34P1.B07 |

GGGAGAGGAGAGAACGTTCTACGAAGAAGATATGAGAGAAAGGATTAAGAGACGCTGTCGATCGATCGATCGATGAAGGGCG

| | |
|---|---|
| SEQ ID No. 357 | ARX34P1.A07 |

GGGAGAGGAGAGAACGTTCTACGAAAAAGATATGAGAGAAAGGATTAAGAGACGCTGTCGASTCGATCGATCGATGAAGGGCG

| | |
|---|---|
| SEQ ID No. 358 | ARX34P1.A01 |

GGGAGAGGAGAGAACGTTCTACGAAAAAGATATGAGAGAAAGGATTAAGAGGCGCTGTCGATCGATCGATCGATGAAGGGCG

| | |
|---|---|
| SEQ ID No. 359 | ARX34P1.G05 |

GGGAGAGGAGAGAACGTTCTACGAAAAAGACATGAGAGAAAGGATTAAGAGACGCTGTCGATCGATCGATCGATGAAGGGCG

| | |
|---|---|
| SEQ ID No. 360 | ARX34P1.F09 |

GGGAGAGGAGAGAACGTTCTACNAAAAAGTATATGAGAGAAAGGATTAANAGACGCTGTCGATCGATCGATCGATGAAGGGCG

| | |
|---|---|
| SEQ ID No. 361 | ARX34P1.B02 |

GGGAGAGGAGAGAACGTTCTACGAAAAAGATATGAGAGAAAAGGATTGAGAGATGCTGTCGATCGATCGATCGATGAAGGGCG

| | |
|---|---|
| SEQ ID No. 362 | ARX34P1.G02 |

GGGAGAGGAGAGCACGTTCTACGAAAAAGATATGGAGAGAAAGGATTAAGAGACGCTGTCGATCGATCGATCGATGAAGGGCG

| | |
|---|---|
| SEQ ID No. 363 | ARX34P1.A04 |

GGGAGAGGAGAGAACGTTCTACGAAAAAGATATGAGAGAAAGGATTAAAAGAGACGCTGTCGATCGATCGATCGATGAAGGGCG

| | |
|---|---|
| SEQ ID No. 364 | ARX34P1.G06 |

GGGAGAGGAGAGAACGTTCTACGAANAAGATACATAGTAGAAAGGATTAATAAGACGCTGTCGATCGATCGATCGATGAAGGGCG

| | |
|---|---|
| SEQ ID No. 365 | ARX34P1.E05 |

GGGAGAGGAGAGAACGTTCTACAGGCGTGTTGGTAGGGTACGACGAGGCATGCGCTGTCGATCGATCGATCGATGAAGGGCG

| | |
|---|---|
| SEQ ID No. 366 | ARX34P1.B11 |

GGGAGAGGAGAGAACGTTCTACGCAAAAATGTGATGCGAGGTAATGGAACGCCGCTGTCGATCGATCGATCGATGAAGGGCG

| | |
|---|---|
| SEQ ID No. 367 | ARX34P1.B01 |

GGGAGAGGAGAGAACGTTCTACGGACCTCAGCGATAGGGGTTGAAACCGACACGCTGTCGATCGATCGATCGATGAAGGGCG

| | |
|---|---|
| SEQ ID No. 368 | ARX34P1.H06 |

GGGAGAGGAGAGAACGTTCTACATGGTCGGATGCTGGGGAGTAGGCAAGGTTCGCTGTCGATCGATCGATCGATGAAGGGCG

| | |
|---|---|
| SEQ ID No. 369 | ARX34P1.C12 |

GGGAGAGGAGAGAACGTTCTACGTATCGGCGAGCGAAGCATCCGGGAGCGTTCGCTGTCGATCGATCGATCGATGAAGGGCG

| | |
|---|---|
| SEQ ID No. 370 | ARX34P1.C09 |

GGGAGAGGAGAGAACGTTCTACGTATTGGCGCGCGAAGCATCCGGGAGCGTTCGCTGTCGATCGATCGATCGATGAAGGGCG

| | |
|---|---|
| SEQ ID No. 371 | ARX34P1.A11 |

GGGAGAGGAGAGAACGTTCTACTTATACCTGACGGCCGGAGGCGCATAGGTGCGCTGTCGATCGATCGATCGATGAAGGGCG

| | |
|---|---|
| SEQ ID No. 372 | ARX34P1.H09 |

GGGAGAGGAGAGAACGTTCTACATGGTCGGATGCTGGGGAGTAGGCATAGGTTCGCTGTCGATCGATCGATCGATGAAGGGCG

| | |
|---|---|
| SEQ ID No. 373 | ARX34P1.B05 |

GGGAGAGGAGAGAACGTTCTACACGAGAGTACTGAGGCGCTTGGTACAGAGTCGCTGTCGATCGATCGATCGATGAAGGGCG

| | |
|---|---|
| SEQ ID No. 374 | ARX34P1.B10 |

GGGAGAGGAGAGAACGTTCTACAGAAGGTAGAAAAAGGATAGCTGTGAGAAGCGCTGTCGATCGATCGATCGATGAAGGGCG

| | |
|---|---|
| SEQ ID No. 375 | ARX34P1.C01 |

GGGAGAGGAGAGAACGTTCTACTGAGGGATAATACGGGTGGGATTGTCTTCCCGCTGTCGATCGATCGATCGATGAAGGGCG

| | |
|---|---|
| SEQ ID No. 376 | ARX34P1.D04 |

GGGAGAGGAGAGAACGTTCTACATTGAGCGTTGAAGTTGGGGAAGCTCCGAGGCCGCTGTCGATCGATCGATCGATGAAGGGCG

| | |
|---|---|
| SEQ ID No. 377 | ARX34P1.E02 |

GGGAGAGGAGAGAACGTTCTACGCGGAGATATACAGCGAGGTAATGGAACGCCGCTGTCAGATCGATCGATCGATGAAGGCG

| | |
|---|---|
| SEQ ID No. 378 | ARX34P1.F01 |

GGGAGAGGAGAGAACGTTCTACGAAGACAGCCCAATAGCGGCACGGAACTTGCGCTGTCGATCGATCGATCGATGAAGGGCG

| | |
|---|---|
| SEQ ID No. 379 | ARX34P1.G03 |

GGGAGAGGAGAGAACGTTCTACCGGTTGAGGGCTCGCGTGGAAGGGCCAACACGCGCTGTCGATCGATCGATCGATGAAGGGCG

| | |
|---|---|
| SEQ ID No. 380 | ARX34P1.H01 |

GGGAGAGGAGAGAACGTTCTACATATCAATAGACTCTTGACGTTTGGGTTTGCGCTGTCGATCGATCGATCGATGAAGGGCG

| | |
|---|---|
| SEQ ID No. 381 | ARX34P1.H02 |

GGGAGAGGAGAGAACGTTCTACAGTGAAGGAAAAGTAAGTGAAGGTGTGCGCTGTCGATCGATCGATCGATGAAGGGCG

| | |
|---|---|
| SEQ ID No. 382 | ARX34P1.H03 |

GGGAGAGGAGAGAACGTTCTACGGATGAAATGAGTGTCTGCGATAGGTTAAGCGCTGTCGATCGATCGATCGATGAAGGGCG

| | |
|---|---|
| SEQ ID No. 383 | ARX34P1.H10 |

GGGAGAGGAGAGAACGTCTACGGAAGGAAATGTGTGTCTGCGATAGGTTAAGCGCTGTCGATCGATCGATCGATGAAGGGCG

### Example 9

### rRmY and rGmH 2′-OMe SELEX™ Against PDGF-BB

Selections were performed to identify aptamers containing 2′-OMe C, U and 2′-OH G, A (rRmY), and the other 2′-O-Methyl A, C, and U and 2′-OH G (rGmH). All selections were direct selections against human PDGF-BB protein target which had been immobilized on a hydrophobic plate. Selections yielded pools significantly enriched for h-_PDGF-BB binding versus naïve, unselected pool. Individual clone sequences for PDGF-BB are reported herein.

Pool Preparation. A DNA template with the sequence 5′-GGGAGAGGAGAGAACGTTCTACNNNNNNNNNNNNNNNNNNNNNNNNNNNNNNC GCTGTCGATCGATCGATCGATG-3′ (SEQ ID NO:459) was synthesized using an ABI EXPEDITE™ DNA synthesizer, and deprotected by standard methods. The templates were amplified with the primers PB.118.95.G5′-GGGAGAGGAGAGAACGTFCTAC-3′ (SEQ ID NO:460) and STC.104.102.A 5′-CATCGATCGATCGATCGACAGC-3′(SEQ ID NO:461) and then used as a template (200 nm template was used for round 1, and for subsequent rounds approximately 50 nM, a 1/10 dilution of an optimized PCR reaction, using conditions described herein, was used) for in vitro transcription with Y639F single mutant T7 RNA polymerase. Transcriptions were done using 200 mM HEPES, 40 mM DTT, 2 mM spermidine, 0.01% TritonX-100, 10% PEG-8000, 5 mM MgCl₂, 1.5 mM MnCl₂, 500 μM NTPs, 500 μM GMP, 0.01 units/μl inorganic pyrophosphatase, and Y639F single mutant T7 polymerase. Two different compositions were transcribed rRmY and rGmH.

Selection. Each round of selection was initiated by immobilizing 20 pmoles of PDGF-BB to the surface Nunc Maxisorp hydrophobic plates for 2 hours at room temperature in 100 μL of 1× Dulbecco's PBS. The supernatant was then removed and the wells were washed 4 times with 120 μL wash buffer (1× DPBS, 0.2% BSA, and 0.05% Tween-20). Pool RNA was heated to 90° C. for 3 minutes and cooled to room temperature for 10 minutes to refold. In round 1, a positive selection step was conducted. Briefly, 1×10¹⁴ molecules (0.2 nmoles) of pool RNA were incubated in 100 μL binding buffer (1× DPBS and 0.05% Tween-20) in the wells with immobilized protein target for 1 hour. The supernatant was then removed and the wells were washed 4× with 120 μL wash buffer. In subsequent rounds a negative selection step was included. The pool RNA was also incubated for 30 minutes at room temperature in empty wells to remove any plastic binding sequences from the pool before the positive selection step. The number of washes was increased after round 4 to increase stringency. In all cases, the pool RNA bound to immobilized PDGF-BB was reverse transcribed directly in the selection plate after by the addition of RT mix (3′ primer, STC.104.102.A, and Thermoscript RT, Invitrogen) followed by incubated at 65° C. for 1 hour. The resulting cDNA was used as a template for PCR (Taq polymerase, New England Biolabs) “Hot start” PCR conditions coupled with a 60° C. annealing temperature were used to minimize primer-dimer formation. Amplified pool template DNA was desalted with a Centrisep column according to the manufacturer's recommended conditions and used to program transcription of the pool RNA for the next round of selection. The transcribed pool was gel purified on a 10% polyacrylamide gel every round.

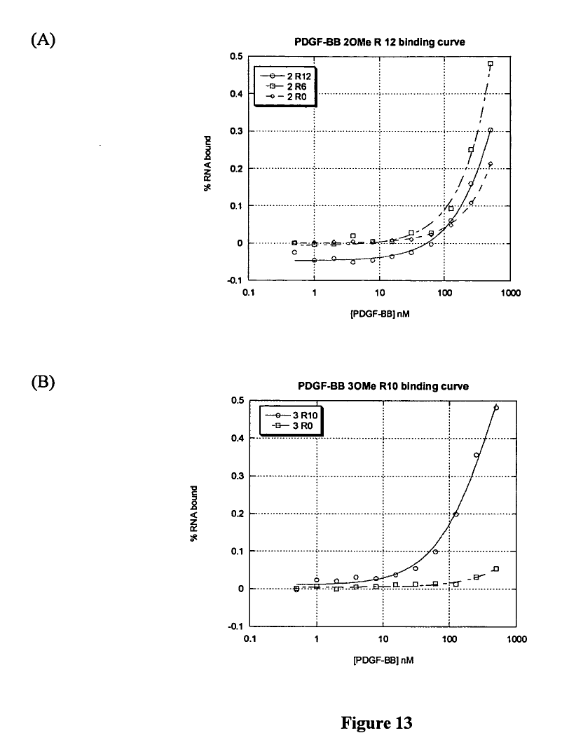

Although the naïve pool does bind to PDGF-BB, the rRmY PDGF-BB selection was enriched after 4 rounds over the naïve pool. The selection stringency was increased and the selection was continued for 8 more rounds. At round 12 the pool is enriched over the naïve pool, but the K_{D} is very high. The rGmH selection was enriched over the naïve pool binding at round 10. The pool K_{D} is also approximately 950 nM or higher. The pools were cloned using TOPO TA cloning kit (Invitrogen) and submitted for sequencing. After 12 rounds of PDGF-BB pool selection clones were transcribed and sequenced. Table 19 shows the clone sequences. FIG. 13(A) shows a binding plot of round 12 pools for rRmY pool PDGF-BB selection and FIG. 13(B) shows a binding plot of round 10 pools for rGmH pool PDGF-BB selection. Dissociation constants were again measured using the sandwich filter binding technique. Dissociation constants (K_{D}s) were estimated fitting the data to the equation: fraction RNA bound=amplitude*K_{D}/(K_{D}+[PDGF-BB]).

TABLE 19

Corresponding cDNAs of the Individual Clone Sequence of rRmY Pool Clones
After Round 12 of Selection to PDGF-BB.

| | |
|---|---|
| SEQ ID No. 384 | PDGF-BB ARX36.SCK.E05 |

GGGAGAGGAGAGAACGTTCTACATCCTGCGTATGATCGGCATCGTAAGACACGCTGTCGATCGATCGATCGATGAAGGGCG

| | |
|---|---|
| SEQ ID No. 385 | PDGF-BB ARX36.SCK.F05 |

GGGAGAGGAGAGAACGTTCTACATCCTTGCGTATGATCGGCATCGTAAGACACGCTGTCGATCGATCGATCGATGAAGGGCG

| | |
|---|---|
| SEQ ID No. 386 | PDGF-BB ARX36.SCK.E01 |

GGGAGAGGAGAGAACGTTCTACGATCGAAGTCGTGACAGAAACCACTCGCTGTCGATCGATCGATCGATGAAGGGCG

| | |
|---|---|
| SEQ ID No. 387 | PDGF-BB ARX36.SCK.F01 |

GGGAGAGGAGAGAACGTTCTACGATCGAAGTCGTGACAGAAACCACTCGCTGTCGATCGATCGATCGATGAAGGGCG

| | |
|---|---|
| SEQ ID No. 388 | PDGF-BB ARX36.SCK.G01 |

GGGAGAGGAGAGAACGTTCTACGGAAAGGTGGCGAAACGAAGAAGATCGCTGTCGATCGATCGATCGATGAAGGGCG

| | |
|---|---|
| SEQ ID No. 389 | PDGF-BB ARX36.SCK.G02 |

GGGAGAGGAGAGAACGTTCTACGGAAAAGGTTGGCGAAACGAAGAANAATTTCGCTGTCGATCGATCGATCGATGAAGGGCG

| | |
|---|---|
| SEQ ID No. 390 | PDGF-BB ARX36.SCK.F04 |

GGGAGAGGAGAGAACGTTCTACTGGGAGTTGCGGTGTTTTGCGGTGGATTTGACGCTGTCGATCGATCGATCGATGAAGGGCG

| | |
|---|---|
| SEQ ID No. 391 | PDGF-BB ARX36.SCK.E04 |

GGGAGAGGAGAGAACGTTCTACTGGGAGTTGCGGTGTTTTGCGGTGGATTTGACGCTGTCGATCGATCGATCGATGAAGGGCG

| | |
|---|---|
| SEQ ID No. 392 | PDGF-BB ARX36.SCK.F02 |

GGGAGAGGAGAGAACGCTCTACAAGATTGTAGATCAACAGCGAAGGCGTGGGCGCTGTCGATCGATCGATCGATGAAGGGCG

| | |
|---|---|
| SEQ ID No. 393 | PDGF-BB ARX36.SCK.E02 |

GGGAGAGGAGAGAACGCTCTACAAGATTGTAGATCAACAGCGAAGGCGTGGGCGCTGTCGATCGATCGATCGATGAAGGGCG

| | |
|---|---|
| SEQ ID No. 394 | PDGF-BB ARX36.SCK.A02 |

GGGAGAGGAGAGAACGTTCTACAAANAAGATNNCCANCNNGAGANAAAGGAGCGCTGTCGATCGATCGATCGATGAAGGGCG

| | |
|---|---|
| SEQ ID No. 395 | PDGF-BB ARX36.SCK.A03 |

GGGAGAGGAGAGAACGTTCTACAAACATCGAAGATCGAACTGAAAAGGAGGGCGCTGTCGATCGATCGATCGATGAAGGGCG

| | |
|---|---|
| SEQ ID No. 396 | PDGF-BB ARX36.SCK.A06 |

GGGAGAGGAGAGAACGTTCTACATGTGCATGCAAGGTGGGGCCTGACACGAGCCGCTGTCGATCGATCGATCGATGAAGGGCG

| | |
|---|---|
| SEQ ID No. 397 | PDGF-BB ARX36.SCK.B01 |

GGGAGAGGAGAGAACGTTCTACAAGGAGTAGATCGACAGAATAGAAAAATCGCTGTCGATCGATCGATCGATGAAGGGCG

| | |
|---|---|
| SEQ ID No. 398 | PDGF-BB ARX36.SCK.B02 |

GGGAGAGGAGAGACGTTCTACAAAAGGTAAGGTCAAAAAAGCGCAAACGTTGACGCTGTCGATCGATCGATCGATGAAGGGCG

| | |
|---|---|
| SEQ ID No. 399 | PDGF-BB ARX36.SCK.D04 |

GGGAGAGGAGAGAACGTTCTACAAAAGGAGGCGAAATAAGTGAGACAATGTGCGCTGTCGATCGATCGATCGATGAAGGGCG

| | |
|---|---|
| SEQ ID No. 400 | PDGF-BB ARX36.SCK.B04 |

GGGAGAGGAGAGAACGTTCTACAAAAATCCACAACATAGCTGTAATTGCTCGCTGTCGATCGATCGATCGATGAAGGGCG

| | |
|---|---|
| SEQ ID No. 401 | PDGF-BB ARX36.SCK.B05 |

GGGAGAGGAGAGACGTTCTACAAGAACATATAACATTTTGGTTGAGAGCAACGCTGTCGATCGATCGATCGATGAAGGGCG

| | |
|---|---|
| SEQ ID No. 402 | PDGF-BB ARX36.SCK.D03 |

GGGAGAGGAGAGAACGTTCTACAAGAGTCNACGATTTCNATCACAAATGTGGCTGCTGTCNATCGATCGATCNATGAAGGGCG

| | |
|---|---|
| SEQ ID No. 403 | PDGF-BB ARX36.SCK.C01 |

GGGAGAGGAGAGAACGTTCTACAAGCAAGCAAAAAAAGTATCGACAGAAGTGGCGCTGTCGATCGATCGATCGATGAAGGGCG

| | |
|---|---|
| SEQ ID No. 404 | PDGF-BB ARX36.SCK.D06 |

GGGAGAGGAGAGAACGTTCTACAAGTAATATCAGAGCAATCGGAATAAGAGTCGCTGTCGATCGATCGATCGATGAAGGGCG

| | |
|---|---|
| SEQ ID No. 405 | PDGF-BB ARX36.SCK.D02 |

GGGAGAGGAGAGAACGTTCTACAGACTTCGATGCGATGGATTTGGAAATGTGCGCTGTCGATCGATCGATCGATGAAGGGCG

| | |
|---|---|
| SEQ ID No. 406 | PDGF-BB ARX36.SCK.C03 |

GGGAGAGGAGAGAACGTTCTACAGAAAGAATTACAGGAACAAATACACGTGCGGCTGTCGATCGATCGATCGATGAAGGGCG

| | |
|---|---|
| SEQ ID No. 407 | PDGF-BB ARX36.SCK.F06 |

GGGAGAGGAGAGAACGTTCTACAGAAATCAATCGAGGTGATCGTTATATAGGCGCTGTCGATCGATCGATCGATGAAGGGCG

| | |
|---|---|
| SEQ ID No. 408 | PDGF-BB ARX36.SCK.C04 |

GGGAGAGGAGAGAACGTTCTACAGATTTGGATCGACAATCTCGTAGAAGAGACGCTGTCGATCGATCGATCGATGAAGGGCG

| | |
|---|---|
| SEQ ID No. 409 | PDGF-BB ARX36.SCK.C06 |

GGGAGAGGAGAGAACGTTCTACAATGCAAGTTTAAGTGTGGTGTCAAACGCACGCTGTCGATCGATCGATCGATGAAGGGCG

| | |
|---|---|
| SEQ ID No. 410 | PDGF-BB ARX36.SCK.G03 |

GGGAGAGGAGAGAACGTTCTACGAAGATGTGTTTAAGAATCGAGGTTTTCGACGCTGTCGATCGATCGATCGATGAAGGGCG

| | |
|---|---|
| SEQ ID No. 411 | PDGF-BB ARX36.SCK.F03 |

GGGAGAGGAGAGAACGTTCTACGAGTTGGCACGCATGTATAGGTATTTTGGCGCTGTCGATCGATCGATCGATGAAGGGCG

| | |
|---|---|
| SEQ ID No. 412 | PDGF-BB ARX36.SCK.C02 |

GGGAGAGGAGAGAACGTTCTACGAGTTGGCACGCATGTATAGGTATTTTGGCGCTGTCGATCGATCGATCGATGAAGGGCG

| | |
|---|---|
| SEQ ID No. 413 | PDGF-BB ARX36.SCK.B03 |

GGGAGAGGAGAGAACGTTCTACGAAAAAAAGAGATGAGAGAAAGGATTAAGAGACGCTGTCGATCGATCGATCGATGAAGGGCG

| | |
|---|---|
| SEQ ID No. 414 | PDGF-BB ARX36.SCK.B06 |

GGGAGAGGAGAGAACGTTCTACGAAAAGGAAAAAAAACGATCGGCAGAGTCCCGCTGTCGATCGATCAGTCGATGAAGGGCG

| | |
|---|---|
| SEQ ID No. 415 | PDGF-BB ARX36.SCK.C05 |

GGGAGAGGAGAGAACGTTCTACGATTAAGGAAACATTTACGCGAATACATGACGCTGTCGATCGATCGATCGATGAAGGGCG

| | |
|---|---|
| SEQ ID No. 416 | PDGF-BB ARX36.SCK.D01 |

GGGAGAGGAGAGAACGTTCTACCGACGTTTGCTCTGAAAATAGGACAGAAGGCGCTGTCGATCGATCGATCGATGAAGGGCG

| | |
|---|---|
| SEQ ID No. 417 | PDGF-BB ARX36.SCK.E03 |

GGGAGAGGAGAGAACGTTCTACGAAGATGTGTTTAAGAATCGAGGTTTTCGACGCTGTCGATCGATCGATCGATGAAGGGCG

| | |
|---|---|
| SEQ ID No. 418 | PDGF-BB ARX36.SCK.A04 |

GGGAGAGGAGAGAACGTTCTACCGAGATCGAAAGGTAAGAGAAAATTCATGGCGCTGTCGATCGATCGATCGATGAAGGGCG

| | |
|---|---|
| SEQ ID No. 419 | PDGF-BB ARX36.SCK.A05 |

GGGAGAGGAGAGAACGTTCTACTAAGATTCGTCGTTCAGACAGAGAAAGCGACGCTGTCGATCGATCGATCGATGAAGGGCG

TABLE 20

Corresponding cDNAs of the Individual Clone Sequence of rGmH Pool Clones After
Round 10 of Selection to PDGF-BB.

SEQ ID No. 420 PDGF-BB ARX36.SCK.E08.M13F
GGGAGAGGAGAGAACGTTCTACCTTGGCGACGATCTGTGACCTGAATTTTTGTCCGCTGTCGATCGATCGATCGATGAAGGGCG
SEQ ID No. 421 PDGF-BB ARX36.SCK.F08.M13F
GGGAGAGGAGAGAACGTTCTACCTTGGCGACGATCTGTGACCTGAATTTTTGTCCGCTGTCGATCGATCGATCGATGAAGGGCG
SEQ ID No. 422 PDGF-BB ARX36.SCK.E09.M13F
GGGAGAGGAGAGAACGTTCTACCTTGGTTCAGCAGCTTTTAACAAAGTATCCCGCTGTCGATCGATCGATCGATGAAGGGCG
SEQ ID No. 423 PDGF-BB ARX36.SCK.F09.M13F
GGGAGAGGAGAGAACGTTCTACCTTGGTCTCAGCAGCTTTAACAAAGTATCCCGCTGTCGATCGATCGATCGATGAAGGGCG
SEQ ID No. 424 PDGF-BB ARX36.SCK.F07.M13F
GGGAGAGAGAGACGTTCTACCGCTATTTTGTTCATTGAGGACTTGTCACGCTGTCGATCGATCGATCGATCGATGAAGGGCG
SEQ ID No. 425 PDGF-BB ARX36.SCK.E07.M13F
GGGAGAGGAGAGAACGTTCTACCGCTATTTTGTTCATTGAAGGACTTGTCACGCTGTCGATCGATCGATCGATGAAGGGCG
SEQ ID No. 426 PDGF-BB ARX36.SCK.E11.M13F
GGGAGAGGAGAGAACGTTCTACCCTATTGAGGTTGATTGGAAGTGCCTATGTCGCTGTCGATCGATCGATCGATGAAGGGCG
SEQ ID No. 427 PDGF-BB ARX36.SCK.F11.M13F
GGGAGAGGAGAGAACGTTCTACCCTATTGAGGTTGATTGGAAGTGCCTATGTCGCTGTCGATCGATCGATCGATGAAGGGCG
SEQ ID No. 428 PDGF-BB ARX36.SCK.F10.M13F
GGGAGAGGAGAGAACGTTCTACTGAAGATGTTATGATGATTGACGAGGAGGCGCTGTCGATCGATCGATCGATGAAGGGCG
SEQ ID No. 429 PDGF-BB ARX36.SCK.E10.M13F
GGGAGAGGAGAGAACGTTCTACTGAAGATGTTATGATGATTGACGAGGAGGCGCTGTCGATCGATCGATCGATGAAGGGCG
SEQ ID No. 430 PDGF-BB ARX36.SCK.E12.M13F
GGGAGAGGAGAGACGTTCTACTGTCTGAGTGTCGCCGCCTTGTGTGATGTTCGCTGTCGATCGATCGATGAATGGGCG
SEQ ID No. 431 PDGF-BB ARX36.SCK.F12.M13F
GGGAGAGGAGAGAACGTTCTACTGTCTGAGTGTCGCCGCCTTGTGTGATGTTCGCTGTCGATCGATCGATCGATGAAGGGCG
SEQ ID No. 432 PDGF-BB ARX36.SCK.A07.M13F
GGAGAGGAGAGAACGTTCTACGTGATGCTGTGAATGAGGTAGTTCGAATACGCTGTCGATCGATCGATCGATGAAGGGCG
SEQ ID No. 433 PDGF-BB ARX36.SCK.C12.M13F
GGGAGAGGAGAGAACGTTCTACGTGAAATCAGGTTGTTAATTTGGGGAATCGCTGTCGATCGATCGATCGATGAAGGGCG
SEQ ID No. 434 PDGF-BB ARX36.SCK.B07.M13F
GGGAGAGGAGAGAACGTTCTACGTATAAGGCCGTAACCGGGTAGCGAGTGGTCGCTGTCGATCGATCGATCGATGAAGGGCG
SEQ ID No. 435 PDGF-BB ARX36.SCK.A09.M13F
GGGAGAGGAGAGAACGTTNTACGTGGGCGAAGGAGCTGCGGGCGTTGNAGTTTGCTGTCGATCGATCGATCGATGAAGGGCG
SEQ ID No. 436 PDGF-BB ARX36.SCK.A11.M13F
GGGAGAGAGAGAACGTTCTACGTCATCCTAGTCTGAGATCGGATTTTCTTGCGCTGTCGATCGATCGATCGATGAAGGGCG
SEQ ID No. 437 PDGF-BB ARX36.SCK.C09.M13F
GGGAGAGGAGAGAACGTTCTACGTTTGCGAGTGTGGTCGACGCTGAATGCGGCGCTGTCGATCGATCGATCGATGAAGGGCG
SEQ ID No. 438 PDGF-BB ARX36.SCK.A08.M13F
GGGAGAGGAGAGAACGTTCTACGGATTGATAGGGATTGAGATGAGGTCTTGTCGCTGTCGATCGATCGATCGATGAAGGGCG
SEQ ID No. 439 PDGF-BB ARX36.SCK.D07.M13F
GGGAGGGAGAGAACGTTCTACGATGTCGTGTTAGATTACTTATTGCTATCTGCTGTCGATCGATCGATCGATGAAGGGCG
SEQ ID No. 440 PDGF-BB ARX36.SCK.D08.M13F
GGGAGAGGAGAGAACGTTCTACGATGCCTGGCGGAACGGAGCCTGGATTTCGCTGTCNATCGATCGATCGATGAAGGGCG
SEQ ID No. 441 PDGF-BB ARX36.SCK.B11.M13F
GGGAGAGGAGAGAACGTTCTACGAGGATTTGACGTGTGTGTGCTAGAGTACGCTGTCGATCGATCGATCGATGAAGGGCG
SEQ ID No. 442 PDGF-BB ARX36.SCK.D09.M13F
GGGAGAGGAGAGAACGTTCTACGAGTATTATGCGTCCCTTGAGGATACACGGCGCTGTCGATCGATCGATGAAGGGCG
SEQ ID No. 443 PDGF-BB ARX36.SCK.B10.M13F
GGGAGAGGAGAGAACGTTCTACAGGGATAACTGTAGCGATGAAAGTAAACGATGCTGTCGATCGATCGATCGATGAAGGGCG
SEQ ID No. 444 PDGF-BB ARX36.SCK.C10.M13F
GGGAGAGGAGAACGTTCTACAAGAAGTGTGGCCGCAGAGACGAAATGCACGCTGTCGATCGATCGATCGATGAAGGGCG
SEQ ID No. 445 PDGF-BB ARX36.SCK.A10.M13F
GGGAGAGGAGAGAACGTTCTACCCATATCTTCCTTATTCCGTTAGTTGCCGCTGTCGATCGATCGATCGATGAAGGGCG
SEQ ID No. 446 PDGF-BB ARX36.SCK.B09.M13F
GGGAGAGGAGAGAACGTTCTACCTGTGTTGATGCTTCCGTTTGAGATTGCCCCGCTGTCGATCGATCGATCGATGAAGGGCG
SEQ ID No. 447 PDGF-BB ARX36.SCK.B12.M13F
GGGAGAGGAGAGAACGTTCTACCNGTAAGANAANCTATTTTAGCCCTTGNNCTGCGCTGTCGATCGATCGATCGATGAAGGGCG
SEQ ID No. 448 PDGF-BB ARX36.SCK.C08.M13F
GGGAGAGGAGAGAACGTTCTACCCTTGTCCTCCAATCCTCTTTTGACTCTTGCCGCTGTCGATCGATCGATCGATGAAGGGCG
SEQ ID No. 449 PDGF-BB ARX36.SCK.D12.M13F
GGGAGAGGAGAGAACGTTCTACCTGATTTTGTCACTGGATTCCGATGGCTTTCGCTGTCGATCGATCGATCGATGAAGGGCG
SEQ ID No. 450 PDGF-BB ARX36.SCK.C11.M13F
GGGGAGGAGAGAACGTTCTACTGTAATAAGGGATGCGTCAGGAACCTGTGTTCGCTGTCGATCGATCGATCGATGAAGGGCG
SEQ ID No. 451 PDGF-BB ARX36.SCK.D11.M13F
GGGAGAGGAGAGAACGTTCTACTGCTTTCCGGGAATTTGTTTGTTTGCTTCCGCTGTCGATCGATCGATCGATGAAGGGCG
SEQ ID No. 452 PDGF-BB ARX36.SCK.C07.M13F
GGGAGAGGAGAGAACGTTCTACTTCGTCGGTTGACTTTTCTTCGTGTAGTGTCGCTGTCGATCGATTGATCGATGAAGGGCG
SEQ ID No. 189 PDGF-BB ARX36.SCK.A12.M13F
GGGAGAGGAGAGAACGTTCTACTATGAAGGGTTTTAAAGATGACACATTAGCCGCTGTCGATCGATCGATCGATGAAGGCG

### Example 10

### C5 Selection with dRmY Pool

Two selections were performed to identify dRmY aptamers to human full length C5 protein. The C5 protein (Quidel Corporation, San Diego, Calif.) was used in full length (“FL”) and partially trypsinized (“TP”) forms and both selections were direct selections against the protein targets which had been immobilized on a hydrophobic plate. Both selections yielded pools significantly enriched for full length C5 binding versus naïve, unselected pool. All sequences shown in this example are shown 5′ to 3′.

Pool Preparation: A DNA template with the sequence CATCGATGATCGATCGATCGACCN30GTAGAACGTTCTCTCCTCTCCCTATAGTGAGT CGTATTA (SEQ ID NO.: 469) was synthesized using an ABI EXPEDITE™ DNA synthesizer, and deprotected by standard methods. The templates were amplified with the primers PB.118.95.G (GGGAGAGGAGAGAACGTTCTAC) (SEQ ID NO.: 470) and PB.118.95.M (CATCGATGATCGATCGATCGACC) (SEQ ID NO.: 471) and then used as a template for in vitro transcription with Y639F single mutant T7 RNA polymerase. Transcriptions were done using 200 mM HEPES, 40 mM DTT, 2 mM spermidine, 0.01% TritonX-100, 10% PEG-8000, 5 mM MgCl₂, 1.5 mM MnCl₂, 500 uM dNTPs, 500 uM GMP, 2 mM spermine, 0.01 units/μl inorganic pyrophosphatase, and Y639F single mutant T7 polymerase.

Selection: In round 1, a positive selection step was conducted on nitrocellulose filter binding columns. Briefly, 1×10¹⁵ molecules (0.5 nmoles) of pool RNA were incubated in 100 μL binding buffer (1× DPBS) with 3 uM full length C5 or 2.6 uM partially trypsinized C5 for 1 hour at room temperature. RNA:protein complexes and free RNA molecules were separated using 0.45 um nitrocellulose spin columns from Schleicher & Schuell (Keene, N.H.). The columns were pre-washed with 1 mL 1× DPBS, and then the RNA:protein containing solutions were added to the columns and spun in a centrifuge at 1500 g for 2 min. Three buffer washes of 1 ml were performed to remove nonspecific binders from the filters, then the RNA:protein complexes attached to the filters were eluted with twice with 200 μl washes of elution buffer (7M urea, 100 mM sodium acetate, 3 mM EDTA, pre-heated to 95° C.). The eluted RNA was precipitated (2 μL glycogen, 1 volume isopropanol, ½ volume ethanol). The RNA was reverse transcribed with the ThermoScript RT-PCR™ system (Invitrogen, Carlsbad, Calif.) according to the manufacturer's instructions, using the 3′ primer described above (PB.18.95.M) followed by PCR amplification (20 mM Tris pH 8.4, 50 mM KCl, 2 mM MgCl₂, 0.5 uM primers PB.118.95.G and PB.118.95.M, 0.5 mM each dNTP, 0.05 units/μL Taq polymerase (New England Biolabs, Beverly, Mass.)). The PCR templates were purified using Centricep columns (Princeton Separations, Princeton, N.J.) and used to transcribe the next round pool.

In subsequent rounds of selection, separation of bound and free RNA was done on Nunc Maxisorp hydrophobic plates (Nunc, Rochester, N.Y.). The round was initiated by immobilizing 20 pmoles of both the full length C5 and partially trypsinized C5 to the surface of the plate for 1 hour at room temperature in 100 μL of 1× DPBS. The supernatant was then removed and the wells were washed 4 times with 120 μL wash buffer (1× DPBS). The protein wells were then blocked with a 1× DPBS buffer containing 0.1 mg/ml yeast tRNA and 0.1 mg/ml salmon sperm DNA as competitors. The pool concentration used was always at least in five fold excess of the protein concentration. The pool RNA was also incubated for 1 hour at room temperature in empty wells to remove any plastic binding sequences, and then incubated in a blocked well with no protein to remove any competitor binding sequences from the pool before the positive selection step. The pool RNA was then incubated for 1 hour at room temperature and the RNA bound to the immobilized C5 was reverse transcribed directly in the selection plate by the addition of RT mix (3′ primer, PB.118.95.M and Thermoscript RT, Invitrogen) followed by incubation at 65° C. for 1 hour. The resulting cDNA was used as a template for PCR (Taq polymerase, New England Biolabs). Amplified pool template DNA was desalted with a Centrisep column (Princeton Separations) according to the manufacturer's recommended conditions and used to program transcription of the pool RNA for the next round of selection. The transcribed pool was gel purified on a 10% polyacrylamide gel every round.

The selection progress was monitored using a sandwich filter binding (dot blot) assay. The 5′-³²P-labeled pool RNA (trace concentration) was incubated with C5, 1× DPBS plus 0.1 mg/mL tRNA and 0.1 mg/mL salmon sperm DNA, for 30 minutes at room temperature, and then applied to a nitrocellulose and nylon filter sandwich in a dot blot apparatus (Schleicher and Schuell). The percentage of pool RNA bound to the nitrocellulose was calculated and monitored approximately every 3 rounds with a single point screen (+/−300 nM C5). Pool K_{d} measurements were measured using a titration of protein and the dot blot apparatus as described above.

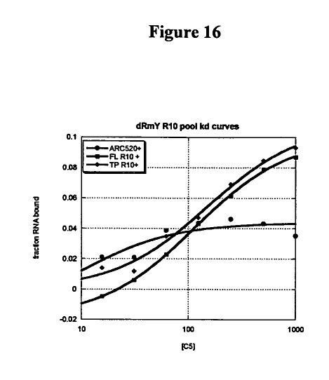

Selection data: Both FL and TP selections were enriched after 10 rounds over the naïve pool. (See FIG. 16). At round 10, the pool K_{d} was approximately 115 nM for the full length and 150 nM for the trypsinized selection, but the extent of binding was only about 10% at the plateau in both. The R10 pools were cloned using TOPO TA cloning kit (Invitrogen) and sequenced.

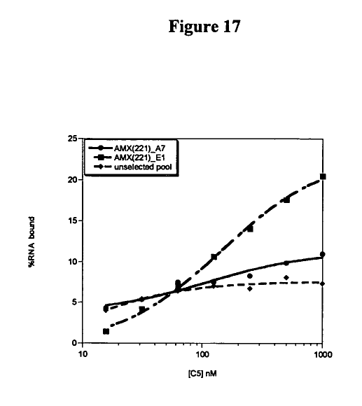

Sequence Information: 45 clones from each pool were sequenced. The R10 full length pool was dominated by one single clone (AMX221.E 1) which made up 24% of the pool, 2 sets of duplicates and single sequences made up the remainder. The R10 trypsinized pool contained 8 copies of the same sequence (AMX221.E1), but the pool was dominated by another sequence (AMX221.A7; 46%). The clone AMX221.E1 had a K_{d} of about 140 nM and the extent of binding increased to 20%. (See FIG. 17).

Unless noted otherwise, individual sequences listed below represent the cDNA clones of the aptamers that were selected under the SELEX conditions provided. The actual aptamers provided in the invention are those corresponding sequences comprising the dRmY combinations of residues, as indicated in the text.

Corresponding cDNA Sequences of the C5 dRmY Sequences:

| | |
|---|---|
| AMX(221)_E1 (SEQ ID No.:472) | GGGAGAGGAGAGAACGTTCTACCTTGGTTTGGCACAGGCATACATACGCAGGGGTC |
| | GATCGATCGATCATCGATG |
| AMX(221)_B3 (SEQ ID No.:473) | GGGAGAGGAGAGAACGTTCTACCTTGGTTTGGCACGGGCATACATACGCAGGGTCG |
| | ATCGATCGATCATCGATG |
| AMX(221)_F11 (SEQ ID No.:474) | GGGAGAGGAGAGAACGTTCTACGGGGAGGTGGGTGGGTAGTGTTGTGTAACGGTCG |
| | ATCGATCGATCATCGATG |
| AMX(221)_C12 (SEQ ID No.:475) | GGGAGAGGAGAGAACGTTCTACTGGCAGGGCATTGAGTAAGGGTGTTGGTGTGGTC |
| | GATCGATCGATCATCGATG |
| AMX(221)_E9 (SEQ ID No.:476) | GGGAGAGGAGAGAACGTTCTACGGATGGTATCGCTGTGCTGATTGGGTGCCAGGTC |
| | GATCGATCGATCATCGATG |
| AMX(221)_A9 (SEQ ID No.:477) | GGGAGAGGAGAGAACGTTCTACAGGAGTGCGATGGGATCAGGTGCGTGCGGGTCGA |
| | TCGATCGATCATCGATG |
| AMX(221)_E8 (SEQ ID No.:478) | GGGAGAGGAGAGAACGTTCTACATCCACCAGCCCGGACATGGCTTGCACGATGGTC |
| | GATCGATCGATCATCGATG |
| AMX(221)_C11 (SEQ ID No.:479) | GGGAGAGGAGAGAACGTTCTACAGCAGGAGAGTGTGTGTGGCAGGGAGATGGGTC |
| | GATCGATCGATCATCGATG |
| AMX(221)_H11 (SEQ ID No.:480) | GGGAGAGGAGAGAACGTTCTACAGGGTGGAAGGATGNGGTACTCNNGGCGTGGGTC |
| | GATCGATCGATCATCGATG |
| AMX(221)_A11 (SEQ ID No.:481) | GGGAGAGGAGAGAACGTTCTACAGATAGGATGGCAAAGGGGGTGTGCAGGCAGGT |
| | CGATCGATCGATCATCGATG |
| AMX(221)_F12 (SEQ ID No.:482) | GGGAGAGGAGAGAACGTTCTACTGACCACGGGGTATGGTTACTGGTTTCTGAGGTC |
| | GATCGATCGATCATCGATG |
| AMX(221)_E11 (SEQ ID No.:483) | GGGAGAGGAGAGAACGTTCTACATGCTGCAATCGAGAGGGGGGCAGTCCACGAGGT |
| | CGATCGATCGATCATCGATG |
| AMX(221)_C9 (SEQ ID No.:484) | GGGAGAGGAGAGAACGTTCTACAGGGCGCTTATGCAATTCACCGGAGGCAAGGGTC |
| | GATCGATCGATCATCGATG |
| AMX(221)_B1 (SEQ ID No.:485) | GGGAGAGGAGAGAACGTTCTACGTAGGGAGGATGGGTGGGGATAGGTGTGCGGGTC |
| | GATCGATCGATCATCGATG |
| AMX(221)_B4 (SEQ ID No.:486) | GGGAGAGGAGAGAACGTTCTACAATGGTGTGTGATTTGAGGGGAGGGTGGTTGGGT |
| | CGATCGATCGATCATCGATG |
| AMX(221)_F3 (SEQ ID No.:487) | GGGAGAGGAGAGAACGTTCTACGATGGAGGAGGAGTACAGGATAGGCTGGATGGT |
| | CGATCGATCGATCATCGATG |
| AMX(221)_G1 (SEQ ID No.:488) | GGGAGAGGAGAGAACGTTCTACTTGTTGTTGTGTGAGTGAGTAGGCTGGCTGGGTCG |
| | ATCGATCGATCATCGATG |
| AMX(221)_A6 (SEQ ID No.:489) | GGGAGAGGAGAGAACGTTCTACGTTTGCGGTCAGGATGGGGTGGTGGGAGGTCGAT |
| | CGATCGATCATCGATG |
| AMX(221)_A5 (SEQ ID No.:490) | GGGAGAGGAGAGAACGTTCTACTTGTGGCAGGCTGCGTACAGGAGCAGATGGTCGA |
| | TCGATCGATCATCGATG |
| AMX(221)_E6 (SEQ ID No.:491) | GGGAGAGGAGAGAACGTTCTACGTTGTGATAGGTTGTGTGAGATGGTGTGCCGGTC |
| | GATCGATCGATCATCGATG |
| AMX(221)_D1 (SEQ ID No.:492) | GGGAGAGGAGAGAACGTTCTACATGTGCAACCAGGAGCAGTAACAGGACAGGTCG |
| | ATCGATCGATCATCGATG |
| AMX(221)_H6 (SEQ ID No.:493) | GGGAGAGGAGAGAACGTTCTACGGTTGGGTGTTGGATGGGCGGTTGGGAGGGTCG |
| | ATCGATCGATCATCGATG |
| AMX(221)_F4 (SEQ ID No.:494) | GGGAGAGGAGAGAACGTTCTACGGGTTGGACAGAGAGAAGGATGAGTACGTGGGT |
| | CGATCGATCGATCATCGATG |
| AMX(221)_D4 (SEQ ID No.:495) | GGGAGAGGAGAGAACGTTCTACGGTAGGTGCTGGGTGCGTAATGGCATCGATGGTC |
| | GATCGATCGATCATCGATG |
| AMX(221)_A4 (SEQ ID No.:496) | GGGAGAGGAGAGAACGTTCTACGGGTGTGTTTGGTGCAAGAGTATTTGTGCGGGTC |
| | GATCGATCGATCATCGATG |
| AMX(221)_H4 (SEQ ID No.:497) | GGGAGAGGAGAGAACGTTCTACAGTGTGCGCTTGGTAATGGTGGTTGGAGTAGGTC |
| | GATCGATCGATCATCGATG |
| AMX(221)_C1 (SEQ ID No.:498) | GGGAGAGGAGAGAACGTTCTACTGGTAGGGATGTGCGTAGAGTTGTCGTGTGGTCG |
| | ATCGATCGATCATCGATG |
| AMX(221)_C2 (SEQ ID No.:499) | GGGAGAGGAGAGAACGTTCTACAACACATCTGGCCATGTCAGTCGAGGATGGTCGA |
| | TCGATCGATCATCGATG |
| AMX(221)_A1 (SEQ ID No.:500) | GGGAGAGGAGAGAACGTTCTACACATGCCGTGCACCCACCACATATCCACAGGTCG |
| | ATCGATCGATCATCGATG |
| AMX(221)_F6 (SEQ ID No.:501) | GGGAGAGGAGAGAACGTTCTACATGCACAACAGCACACACGTGGCATCGATGGTCG |
| | ATCGATCGATCATCGATG |

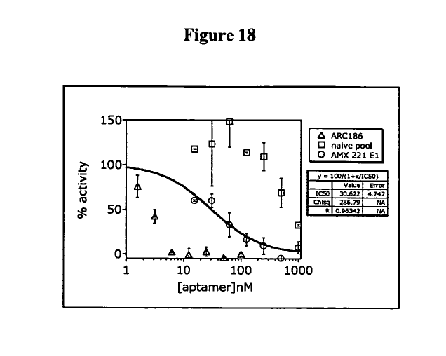

Hemolysis Assay: The effect of the AMX221.E1 clone on the classical pathway of the complement system was analyzed using a hemolysis assay compared to both ARC186 (Anti-C5 aptamer, positive control) and unselected dRmY pool (negative control). In the assay of hemolytic inhibition, a solution of 0.2% whole human serum was mixed with antibody-coated sheep erythrocytes (Diamedix EZ Complement CH50 Test, Diamedix Corporation, Miami, Fla.) in the presence oftitrated AMX221.E1. The assay was run in veronal-buffered saline containing calcium, magnesium and 1% gelatin (GVB⁺⁺ complement buffer) and incubated for 1 hr at 25° C. After incubation the samples were centrifuged. The optical density at 415 nm (OD₄₁₅) of the supernatant was read. The inhibition of hemolysis activity is expressed as % hemolysis activity compared to control. See FIG. 18. The IC₅₀ of the clone was calculated to be about 30 nM.

### Example 11

### IFN-γ Selection with dRmY Pool

A selection was performed to identify IFN-γ aptamers containing deoxy-A,G and 2′0-Methyl C, U residues (dRmY composition). This was a direct selection against h-IFN-γ (R&D Systems, Minneapolis, Minn.) which had been immobilized on a hydrophobic plate. This selection yielded a pool enriched for hIFN-γ binding versus naïve, unselected pool. All sequences shown in this example are shown 5′ to 3′.

Pool Preparation: A synthetic dRmY pool (ARC520) with the sequence GGGAGAGGAGAGAACGUUCUAC-N30-GGUCGAUCGAUCGAUCAUCGAUG (SEQ ID NO.: 502) was synthesized using an ABI EXPEDITE™ DNA synthesizer, and deprotected by standard methods.

Selection: Each round of selection was initiated by immobilizing 20 pmoles of hIFN-γ to the surface of a Nunc Maxisorp hydrophobic plate for 1 hour at room temperature in 100 μL of 1× Dulbecco's PBS ((DPBS) 0.901 mM CaCl₂, 0.493 mM MgCl₂-6H₂O, 2.67 mM KCl, 1.47 mM KH₂PO₄, 137.93 mM NaCl, 8.06 mM Na₂HPO₄-7H₂O). The supernatant was removed and the wells were washed 3 times with 120 μL wash buffer (1× DPBS). The target-immobilized wells were then blocked for 1 hour at room temperature in 100 μl blocking buffer (1× DPBS and 0.1 mg/ml BSA) then washed 3 times with 1× DPBS. In round one, 500 pmoles of pool RNA (3×10¹⁴ molecules) was split into 3 wells of immobilized protein target and incubated for 1 hour in 100 μL DPBS plus 0.1 mg/ml tRNA and 0.1 mg/ml salmon sperm DNA (ssDNA). All subsequent rounds were started with 100 pmoles of pool RNA in 100 μl 1× DPBS in 1 well of immobilized target. Beginning in round 2, a negative selection was added in which the pool RNA was also incubated for 1 hour at room temperature in empty wells to remove any plastic binding sequences from the pool before the positive selection step. Beginning in round 3, a second negative selection step was introduced; the pool was incubated for 1 hour in a well that had been previously blocked with 100 μl blocking buffer (1× DPBS and 0.1 mg/ml BSA). After the positive incubation, the wells were washed 3 times with 120 μL wash buffer. The reverse transcription reaction was added directly in the selection plate (1.75 uM 3′ primer, (KMT.108.59.B CATCGATGATCGATCGATCGAC) (SEQ ID NO.: 503), 1 mM dNTP's, 1× cDNA synthesis buffer, 5 mM DTT, and 75 units/μl Thermoscript RT, (Invitrogen, Carlsbad, Calif.) followed by incubation at 65° C. for 30 minutes. The resulting cDNA was used as a template for PCR (20 mM Tris pH 8.4, 50 mM KCl, 2 mM MgCl₂, 0.5 uM primers KMT.108.59.B and KMT.108.59.A (TAATACGACTCACTATAGGGAGAGGAGAGAACGTTCTAC) (SEQ ID NO.: 504), 0.5 mM each dNTP, 0.05 units/μL Taq polymerase (New England Biolabs, Beverly, Mass.). Amplified pool PCR was desalted with a Micro Bio-Spin column (Bio-Rad, Hercules, Calif.) or Centricep spin columns (Princeton Separations, Princeton, N.J.) according to the manufacturer's recommended conditions and then used as a template for in vitro transcription with T7 RNA polymerase (Y639F). Transcriptions were done using 200 mM HEPES, 40 mM DTT, 2 mM spermidine, 0.01% TritonX-100, 10% PEG-8000, 9.6 mM MgCl₂, 2.9 mM MnCl₂, 30 μM GTP, 2 mM mCTP, 2 mM mUTP, 2 mM dGTP, 2 mM dATP, 2 mM GMP, 2 mM spermine, 0.01 units/μl inorganic pyrophosphatase, and T7 polymerase (Y639F). The transcribed pool was gel purified using a 10% polyacrylamide gel in each round.

After 10 rounds of selection, the pool was split and carried forward using 2 different selection buffers. The first selection buffer was as described above. In the second selection buffer the NaCl concentration in the DPBS was increased to 250 mM to increase stringency. The selection steps were as described above but for the change in buffer.

The selection progress was monitored using a sandwich filter binding assay. The 5′-³²P-labeled pool RNA (trace concentration) was incubated with hIFN-γ, 1× DPBS plus 0.1 mg/ml tRNA, 0.1 mg/ml ssDNA, and 0.1 mg/ml BSA, for 30 minutes at room temperature and then applied to a nitrocellulose and nylon filter sandwich in a dot blot apparatus (Schleicher and Schuell, Keene, N.H.). The percentage of pool RNA bound to the nitrocellulose was calculated after round 5, 7, 9 and 10 and 12 with a 2 point screen (100 nM and 300 nM hIFN-γ). Pool K_{d} measurements were measured using a titration of protein and the dot blot apparatus as described above.

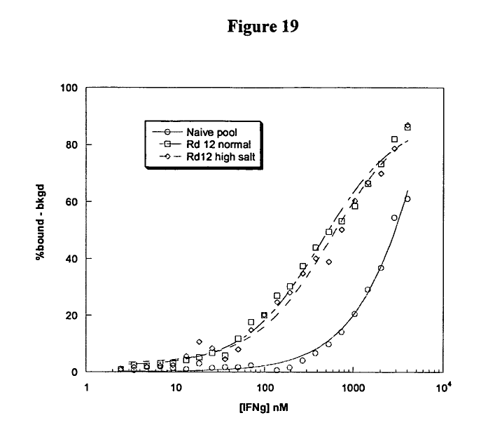

The dRmY hIFN-γ selection was enriched for hIFN-γ binding vs. the naïve pool after 10 rounds of selection. Enrichment after 12 rounds is shown in FIG. 19. The pool K_{d}'s for Round 10 were 605 nM for the normal stringency selection and 675 nM for the high salt selection. The Round 12 pool K_{d}'s were 445 nM for the normal stringency selection and 590 nM for the high salt selection. Additional rounds of selection did not improve the pool K_{d}. The Round 10, 12 and 15 pools were cloned using TOPO TA cloning kit (Invitrogen) and individual sequences were generated. There were 3 dominant clones and the rest were single sequences.

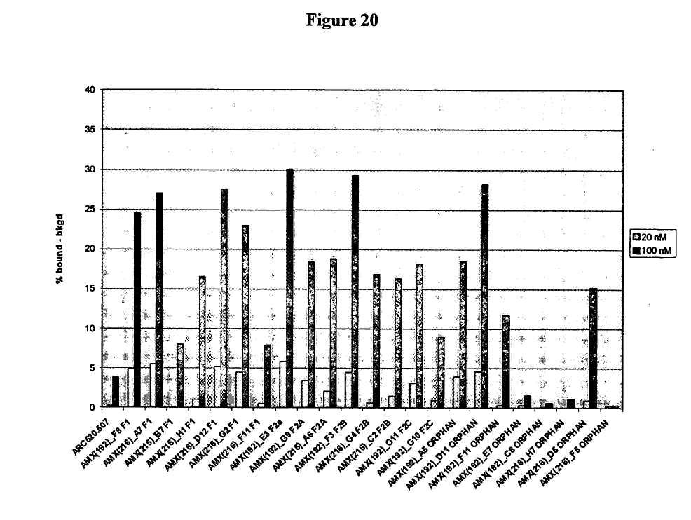

Clone screening: A 2 point screen (20 nM and 100 nM) was done with γ-³²P ATP labeled clones from Round 10 and Round 12 as described above. See FIG. 20.

Five clones were picked for further characterization by K_{d} (see Table 21) which were determined using the dot blot assay and buffer conditions of 1× Dulbecco's PBS and 0.1 mg/ml BSA.

TABLE 21
dRmY IFNg binders

| | | Filter |
|---|---|---|
| ARC # (SEQ_Name) | K_{d} | bkgd |
| ARC789 (AMX(192)_A5) | 167.31 | 10.52578 |
| ARC818 (AMX(192)_E3) | 227.87 | 5.599839 |
| ARC819(AMX(192)_F3) | 206 | 7.346605 |
| ARC820(AMX(192)_D11) | 169.28 | 19.17767 |
| ARC821(AMX(216)_A7) | 97 | 6.090329 |

Unless noted otherwise, individual sequences listed below represent the cDNA clones of the aptamers that were selected under the SELEX conditions provided. The actual aptamers provided in the invention are those corresponding sequences comprising the dRmY combinations of residues, as indicated in the text.

Corresponding cDNA Sequences of the dRmY Sequences from Round 10, 12 and 15 Pools Clones Tested for Binding: clones with K_{d} values are in bold:

| | |
|---|---|
| AMX(192)_B5 (SEQ ID NO.:505) | GGGAGAGGAGAGAACGTTCTACGGGGGTCGTGGGAGTAAGGGGG |
| | TGTAGGTAGGTCGATCGATCGATCATCGATG |
| AMX(192)_G10 (SEQ ID NO.:506) | GGGAGAGGAGAGAACGTTCTACGGGTGGATGGGAGGGGGACAGGT |
| | AGGATGGGGTCGATCGATCGATCATCGATG |
| AMX(192)_F8 (SEQ ID NO.:507) | GGGAGAGGAGAGAACGTTCTACGGGGGTCGTGGGAGTAAGGGGG |
| | TGTAGGTAGGTCGATCGATCGATCATCGATG |
| AMX(192)_E3 (ARC818) (SEQ ID NO.:508) | GGGAGAGGAGAGAACGTTCTACGGGTGGCTGGGGCAGGGGAGGTA |
| | GGTAGGGTCGATCGATCGATCATCGATG |
| AMX(192)_G11 (SEQ ID NO.:509) | GGGAGAGGAGAGAACGTTCTACGGGTGGATGGGAGGGGGACAGGC |
| | AGGATGGGGTCGATCGATCGATCATCGATG |
| AMX(192)_G9 (SEQ ID NO.:510) | GGGAGAGGAGAGAACGTTCTACGGGTGGTTGGGAAGGGGGATGGA |
| | GGTATGGGGTCGATCGATCGATCATCGATG |
| AMX(192)_A5 (ARC789) (SEQ ID NO.:511) | GGGAGAGGAGAGAACGTTCTACGTTTGCGGTCAGGATGGGGTGGT |
| | GGGAGGTCGATCGATCGATCATCGATG |
| AMX(192)_F3 (ARC819) (SEQ ID NO.:512) | GGGAGAGGAGAGAAACGTTCTACGGGCGGTTGGTCGGGGAGGATGGT |
| | ACAGGGTCGATCGATCGATCATCGATG |
| AMX(192)_D11 (ARC820) (SEQ ID NO.:513) | GGGAGAGGAGAGAACGTTCTACGGGGAGGAGGGTGGGGTAGCAGG |
| | TGTGGCAGGTCGATCGATCGATCATCGATG |
| AMX(192)_F11 (SEQ ID NO.:514) | GGGAGAGGAGAGAACGTTCTACTCGGGTGGGGGGGCAGCAAGGT |
| | AGCTGTAGGTCGATCGATCGATCATCGATG |
| AMX(216)_A7 (ARC821) (SEQ ID NO.:515) | GGGAGAGGAGAGAACGTTCTACGGGGGTCGTGGGAGTAAGGGGG |
| | TGTAGGTAGGTCGATCGATCGATCATCGATG |
| AMX(216)_D5 (SEQ ID NO.:516) | GGGAGAGGAGAGAACGTTCTACGATGGGCGGATGGTGGGAGGAT |
| | GGGCAATAGGTCGATCGATCGATCATCGATG |
| AMX(216)_B7 (SEQ ID NO.:517) | GGGAGAGGAGAGAACGTTCTACGGGGGTCGTGGGAGTAAGGGGG |
| | TGTAGGTAGGTCGATCGATCGATCATCGATG |
| AMX(216)_H1 (SEQ ID NO.:518) | GGGAGAGGAGAGAACGTTCTACGGGGGTCGTGGGAGTAAGGGGGG |
| | TGTAGGTAGGTCGATCGATCGATCATCGATG |
| AMX(216)_D12 (SEQ ID NO.:519) | GGGAGAGGAGAGAACNTTCTACCGGGGTCGTGGGAGTAAGGGGG |
| | TGTAGGTAGGTCNATCNATCNATCNTCNATG |
| AMX(216)_G2 (SEQ ID NO.:520) | GGGAGAGGAGAGAACGTTCTACGGGGGTCGTGGGAGAAAGGGGG |
| | TGTAGGTAGGTCGATCGATCGATCATCGATG |
| AMX(216)_G4 (SEQ ID NO.:521) | GGGAGAGGAGAGAACGTTCTACGGGCGGTGGGGGTCGGGGAGGATGGT |
| | ACAGGGTCGATCGATCGATCATCGATG |
| AMX(216)_A6 (SEQ ID NO.:522) | GGGAGAGGAGAGAACGTTCTACGGGTGGTTGGGGCAGGGGAGGTA |
| | GGTAGGGTCGATCGATCGATCATCGATG |

Clone Minimization: Clones AMX(192)_E3 and AMX(192)_F3 were minimized based on a putative G-quartet structure (ARC872 and ARC873 respectively). These minimized aptamers were assayed in the hIFN-γ ELISA described below.

Minimers of AMX(192)_E3 and AMX(192)_F3

| | |
|---|---|
| ARC872 (SEQ ID NO.:523) | GGGCGGUUGGGGUCGGGGAGGAUGGUACAGGG |
| ARC873 (SEQ ID NO.:524) | GGGUGGCUGGGGCAGGGGAGGUAGGUAGGG |

IFN-γ ELISA: The following ELISA method was used to measure the ability of IFN-γ aptamers to inhibit hIFN-γ from binding to the IFNγ-R1 receptor. To capture the IFNγ-R1, 175 ng of IFNγ-R1 (R&D systems, Minneapolis, Minn.) in 100 μl of PBS (pH 7.4) was incubated in each well of a Nunc Maxisorb plate (Nunc, Rochester, N.Y.) for 2 hours at room temperature. The solution was discarded and the plate was washed 3 times with 200 μl of TBS-T (25 mM Tris-HCl pH 7.5, 150 mM NaCl and 0.01% Tween −20). The plate was then blocked with 200 μl of 5% nonfat dry milk in TBS-T for 30 minutes at room temperature. After blocking, the plate was washed 3 times with 200 μl of TBS-T. Then, 100 μl of various concentrations of aptamers mixed with 5 nmoles of IFN-γ (R & D Systems) were incubated in appropriate wells for 1.5 hours at room temperature. The plate was then washed 3 times with 200 μl of TBS-T, then 100 μl of monoclonal antibody against IFN-γ (1:2000) (Biosource, Camarillo, Calif.) was added and incubated for 1 hour at room temperature. After incubation with the monoclonal antibody, the plate was washed 3 times with 200 μl of TBS-T, then 100 μl of HRP linked rabbit-anti-mouse antibody (1:4000 Cell Signalling Technology, Beverly, Mass.) was added for 0.5 hours at room temperature. After incubation with the secondary antibody, the plate was washed 3 times with 200 μl of TBS-T, then 100 μl of 1-Step Ultra TMB-ELISA solution (Pierce, Rockford, Ill.) was added and incubated in the dark at room temperature for 5 minutes. Subsequently, 100 μl of 2 N H₂SO₄ was added to stop the reaction and the plate was read in a SpectraMax 96 well plate reader at 450 run.

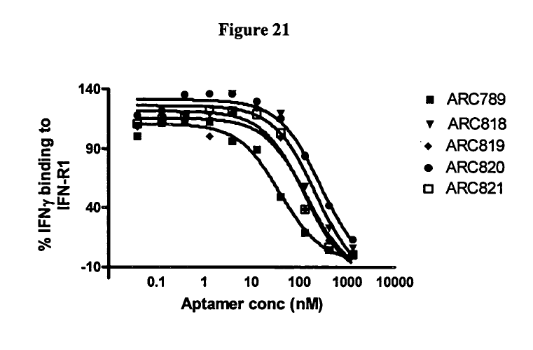

IFNγ-R1 Binding Inhibition with hIFN-γ Aptamers: Five full length and 2 minimized aptamers to IFN-γ were tested for receptor binding inhibition activity using the ELISA method described above. A titration of each aptamer was tested in duplicate (assay performed twice, on 2 separate days). Examples of the IC₅₀ curves generated are shown in FIG. 21. IC₅₀'s for the duplicate assays were calculated and are shown in Table 22 below along with K_{d} values for each of the respective aptamers.

TABLE 22
K_{d} and IC50 values for hIFN-γ aptamers.

| | K_{d} (nM) | IC50 (nM) - Day 1 | IC50 nM - Day 2 |
|---|---|---|---|
| ARC789 | 150 | 40 | 70 |
| ARC818 | 180 | 220 | 190 |
| ARC819 | 180 | 140 | 160 |
| ARC820 | 170 | 280 | 270 |
| ARC821 | 140 | 130 | 100 |
| ARC872 | Not tested | Not tested | 200 |
| ARC873 | Not tested | Not tested | 330 |

The present invention having been described by detailed description and the foregoing non-limiting examples, is now defined by the spirit and scope of the following claims.

1. A method for identifying nucleic acid ligands comprising a modified nucleotide to a target molecule comprising:
a) preparing a transcription reaction mixture comprising a mutated polymerase, one or more 2′-modified nucleotide triphosphates (NTPs), magnesium ions and one or more oligonucleotide transcription templates;
b) preparing a candidate mixture of single-stranded nucleic acids by transcribing the one or more oligonucleotide transcription templates under conditions whereby the mutated polymerase incorporates at least one of the one or more modified nucleotides into each nucleic acid of said candidate mixture, wherein each nucleic acid of said candidate mixture comprises a 2′-modified nucleotide selected from the group consisting of a 2′-position modified pyrimidine and a 2′-position modified purine;
c) contacting the candidate mixture with said target molecule;
d) partitioning the nucleic acids having an increased affinity to the target molecule relative to the candidate mixture from the remainder of the candidate mixture; and
e) amplifying the increased affinity nucleic acids, in vitro, to yield a ligand-enriched mixture of nucleic acids, whereby nucleic acid ligands of the target molecule are identified.

2. The method of claim 1, wherein the one or more 2′-modified nucleotides are selected from the group consisting of 2′-OH, 2′-deoxy, 2′-O-methyl, 2′-NH_{2,2}′-F, and 2′-methoxy ethyl modifications.

3. The method of claim 1, wherein the one or more 2′-modified nucleotides are a 2′-O-methyl modification.

4. The method of claim 1, wherein the one or more 2′-modified nucleotides are a 2′-F modification.

5. The method of claim 1, wherein the mutated polymerase is a mutated T7 RNA polymerase.

6. The method of claim 5, wherein the mutated T7 RNA polymerase comprises a mutation at position 639 from a tyrosine residue to a phenylalanine residue (Y639F).

7. The method of claim 5, wherein the mutated T7 RNA polymerase comprises a mutation at position 784 from a histidine residue to an alanine residue (H784A).

8. The method of claim 5, wherein the mutated T7 RNA polymerase comprises a mutation at position 639 from a tyrosine residue to a phenylalanine residue and a mutation at position 784 from a histidine residue to an alanine residue (Y639F/H784A).

9. The method of claim 1, wherein the oligonucleotide transcription template further comprises a leader sequence incorporated into a fixed region at the 5′ end of the oligonucleotide transcription template.

10. The method of claim 9, wherein the leader sequence comprises an all-purine leader sequence.

11. The method of claim 10, wherein the all-purine leader sequence has a length selected from the group consisting of at least 6 nucleotides long; at least 8 nucleotides long; at least 10 nucleotides long; at least 12 nucleotides long; and at least 14 nucleotides long.

12. The method of claim 1, wherein the transcription reaction mixture further comprises manganese ions.

13. The method of claim 12, wherein the concentration of magnesium ions is between 3.0 and 3.5 times greater than the concentration of manganese ions.

14. The method of claim 1, wherein each NTP is present at a concentration of 0.5 mM, the concentration of magnesium ions is 5.0 mM, and the concentration of manganese ions is 1.5 mM.

15. The method of claim 1, wherein each NTP is present at a concentration of 1.0 mM, the concentration of magnesium ions is 6.5 mM, and the concentration of manganese ions is 2.0 mM.

16. The method of claim 1, wherein each NTP is present at a concentration of 2.0 mM, the concentration of magnesium ions is 9.6 mM, and the concentration of manganese ions is 2.9 mM.

17. The method of claim 1, wherein the transcription reaction mixture further comprises 2′-OH GTP.

18. The method of claim 1, wherein the transcription reaction mixture further comprises a polyalkylene glycol.

19. The method of claim 18, wherein the polyalkylene glycol is polyethylene glycol (PEG).

20. The method of claim 1, wherein the transcription reaction mixture further comprises GMP.

21. The method of claim 1 further comprising
f) repeating steps d) and e).

22. A nucleic acid ligand to thrombin identified according to the method of claim 1.

23. A nucleic acid ligand to vascular endothelial growth factor (VEGF) identified according to the method of claim 1.

24. A nucleic acid ligand to IgE identified according to the method of claim 1.

25. A nucleic acid ligand to IL-23 identified according to the method of claim 1.

26. A nucleic acid ligand to platelet-derived growth factor-BB (PDGF-BB) identified according to the method of claim 1.

27. A nucleic acid ligand to C5 identified according to the method of claim 1.

28. A nucleic acid ligand to interferon gamma (IFN-γ) identified according to the method of claim 1.

29. The method of claim 1, wherein the 2′ modified nucleotide triphosphates comprise a mixture of 2′-OH adenosine triphosphate (ATP), 2′-OH guanosine triphosphate (GTP), 2′-O-methyl cytidine triphosphate (CTP) and 2′-O-methyl uridine triphosphate (UTP).

30. The method of claim 1, wherein the 2′ modified nucleotide triphosphates comprise a mixture of 2′-deoxy purine nucleotide triphosphates and 2′-O-methylpyrimidine nucleotide triphosphates.

31. The method of claim 1, wherein the 2′ modified nucleotide triphosphates comprise a mixture of 2′-O-methyl adenosine triphosphate (ATP), 2′-OH guanosine triphosphate (GTP), 2′-O-methyl cytidine triphosphate (CTP) and 2′-O-methyl uridine triphosphate (UTP).

32. The method of claim 1, wherein the 2′ modified nucleotide triphosphates comprise a mixture of 2′-O-methyl adenosine triphosphate (ATP), 2′-O-methyl cytidine triphosphate (CTP) and 2′-O-methyl uridine triphosphate (UTP), 2′-O-methyl guanosine triphosphate (GTP) and deoxy guanosine triphosphate (GTP), wherein the deoxy guanosine triphosphate comprises a maximum of 10% of the total guanosine triphosphate population.

33. The method of claim 1, wherein the 2′ modified nucleotide triphosphates comprise a mixture of 2′-O-methyl adenosine triphosphate (ATP), 2′-F guanosine triphosphate (GTP), 2′-O-methyl cytidine triphosphate (CTP) and 2′-O-methyl uridine triphosphate (UTP).

34. The method of claim 1, wherein the 2′ modified nucleotide triphosphates comprise a mixture of 2′-deoxy adenosine triphosphate (ATP), 2′-O-methyl guanosine triphosphate (GTP), 2′-O-methyl cytidine triphosphate (CTP)and 2′-O-methyl uridine triphosphate (UTP).

35. A method of preparing a nucleic acid comprising one or more modified nucleotides comprising:
(a) preparing a transcription reaction mixture comprising a mutated polymerase, one or more 2′-modified nucleotide triphosphates (NTPs), magnesium ions and one or more oligonucleotide transcription templates; and
(b) contacting the one or more oligonucleotide transcription templates with the mutated polymerase under conditions whereby the mutated polymerase incorporates the one or more 2′-modified nucleotides into a nucleic acid transcription product.

36. The method of claim 35, wherein the one or more 2′-modified nucleotides are selected from the group consisting of 2′-OH, 2′-deoxy, 2′-O-methyl, 2′-NH_{2,2}′-F, and 2′-methoxy ethyl modifications.

37. The method of claim 35, wherein the one or more 2′-modified nucleotides are a 2′-O-methyl modification.

38. The method of claim 35, wherein the one or more 2′-modified nucleotides are a 2′-F modification.

39. The method of claim 35, wherein the mutated polymerase is a mutated T7 RNA polymerase.

40. The method of claim 39, wherein the mutated T7 RNA polymerase comprises a mutation at position 639 from a tyrosine residue to a phenylalanine residue (Y639F).

41. The method of claim 39, wherein the mutated T7 RNA polymerase comprises a mutation at position 784 from a histidine residue to an alanine residue (H784A).

42. The method of claim 39, wherein the mutated T7 RNA polymerase comprises a mutation at position 639 from a tyrosine residue to a phenylalanine residue and a mutation at position 784 from a histidine residue to an alanine residue (Y639F/H784A).

43. The method of claim 35, wherein the oligonucleotide transcription template further comprises a leader sequence incorporated into a fixed region at the 5′ end of the oligonucleotide transcription template.

44. The method of claim 43, wherein the leader sequence comprises an all-purine leader sequence.

45. The method of claim 44, wherein the all-purine leader sequence has a length selected from the group consisting of at least 6 nucleotides long; at least 8 nucleotides long; at least 10 nucleotides long; at least 12 nucleotides long; and at least 14 nucleotides long.

46. The method of claim 35, wherein the transcription reaction mixture further comprises manganese ions.

47. The method of claim 46, wherein the concentration of magnesium ions is between 3.0 and 3.5 times greater than the concentration of manganese ions.

48. The method of claim 35, wherein each NTP is present at a concentration of 0.5 mM each, the concentration of magnesium ions is 5.0 mM, and the concentration of manganese ions is 1.5 mM.

49. The method of claim 35, wherein each NTP is present at a concentration of 1.0 mM each, the concentration of magnesium ions is 6.5 mM, and the concentration of manganese ions is 2.0 mM.

50. The method of claim 5, wherein each NTP is present at a concentration of 2.0 mM each, the concentration of magnesium ions is 9.6 mM, and the concentration of manganese ions is 2.9 mM.

51. The method of claim 35, wherein the transcription reaction mixture further comprises 2′-OH GTP.

52. The method of claim 35, wherein the transcription reaction mixture further comprises a polyalkylene glycol.

53. The method of claim 52, wherein the polyalkylene glycol is polyethylene glycol (PEG).

54. The method of claim 35, wherein the transcription reaction mixture further comprises GMP.

55. An aptamer composition comprising a sequence where substantially all adenosine nucleotides are 2′-OH adenosine, substantially all guanosine nucleotides are 2′-OH guanosine, substantially all cytidine nucleotides are 2′-O-methyl cytidine, and substantially all uridine nucleotides are 2′-O-methyl uridine.

56. The aptamer composition of claim 55, wherein said aptamer comprises a sequence composition where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine.

57. The aptamer composition of claim 55, wherein said aptamer comprises a sequence composition where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine.

58. The aptamer composition of claim 55, wherein said aptamer comprises a sequence composition where 100% of all adenosine nucleotides are 2′-OH adenosine, at 100% of all guanosine nucleotides are 2′-OH guanosine, 100% of all cytidine nucleotides are 2′-O-methyl cytidine and 100% of all uridine nucleotides are 2′-O-methyl uridine.

59. An aptamer composition comprising a sequence where substantially all purine nucleotides are 2′-deoxy purines and substantially all pyrimidine nucleotides are 2′-O-methyl pyrimidines.

60. The aptamer composition of claim 59, wherein said aptamer comprises a sequence composition where at least 80% of all purine nucleotides are 2′-deoxy purines and at least 80% of all pyrimidine nucleotides are 2′-O-methyl pyrimidines.

61. The aptamer composition of claim 59, wherein said aptamer comprises a sequence composition where at least 90% of all purine nucleotides are 2′-deoxy purines and at least 90% of all pyrimidine nucleotides are 2′-O-methyl pyrimidines.

62. The aptamer composition of claim 59, wherein said aptamer comprises a sequence composition where 100% of all purine nucleotides are 2′-deoxy purines and 100% of all pyrimidine nucleotides are 2′-O-methyl pyrimidines

63. An aptamer composition comprising a sequence composition where substantially all guanosine nucleotides are 2′-OH guanosine, substantially all cytidine nucleotides are 2′-O-methyl cytidine, substantially all uridine nucleotides are 2′-O-methyl uridine, and substantially all adenosine nucleotides are 2′-O-methyl adenosine.

64. The aptamer composition of claim 63, wherein said aptamer comprises a sequence composition where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine.

65. The aptamer composition of claim 63, wherein said aptamer comprises a sequence composition where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine.

66. The aptamer composition of claim 63, wherein said aptamer comprises a sequence composition where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

67. An aptamer composition comprising a sequence where substantially all adenosine nucleotides are 2′-O-methyl adenosine, substantially all cytidine nucleotides are 2′-O-methyl cytidine, substantially all guanosine nucleotides are 2′-O-methyl guanosine or deoxy guanosine, substantially all uridine nucleotides are 2′-O-methyl uridine, wherein less than about 10% of the guanosine nucleotides are deoxy guanosine.

68. The aptamer composition of claim 67, wherein said aptamer comprises a sequence composition where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are deoxy guanosine.

69. The aptamer composition of claim 67, wherein said aptamer comprises a sequence composition where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are deoxy guanosine.

70. The aptamer composition of claim 67, wherein said aptamer comprises a sequence composition where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine and no more than about 10% of all guanosine nucleotides are deoxy guanosine.

71. An aptamer composition comprising a sequence where substantially all adenosine nucleotides are 2′-O-methyl adenosine, substantially all uridine nucleotides are 2′-O-methyl uridine, substantially all cytidine nucleotides are 2′-O-methyl cytidine, and substantially all guanosine nucleotides are 2′-F guanosine sequence.

72. The aptamer composition of claim 71, wherein said aptamer comprises a sequence composition where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine.

73. The aptamer composition of claim 71, wherein said aptamer comprises a sequence composition where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine

74. The aptamer composition of claim 71, wherein said aptamer comprises a sequence composition where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

75. An aptamer composition comprising a sequence where substantially all adenosine nucleotides are 2′-deoxy adenosine, substantially all cytidine nucleotides are 2′-O-methyl cytidine, substantially all guanosine nucleotides are 2′-O-methyl guanosine, and substantially all uridine nucleotides are 2′-O-methyl uridine.

76. The aptamer composition of claim 75, wherein said aptamer comprises a sequence composition where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine.

77. The aptamer composition of claim 75, wherein said aptamer comprises a sequence composition where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine.

78. The aptamer composition of claim 75, wherein said aptamer comprises a sequence composition where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.


SEQUENCE LISTING (524 sequences)

SEQ ID NO: 1 - DNA, 93 bp, Artificial
  aptamer library template ARC254
catcgatgctagtcgtaacgatccnnnnnnnnnnnnnnnnnnnnnnnnnnnnnncgagaacgttctctcctctccctatagtgagtcgtatta

SEQ ID NO: 2 - DNA, 92 bp, Artificial
  aptamer library template ARC255
catgcatcgcgactgactagccgnnnnnnnnnnnnnnnnnnnnnnnnnnnnnngtagaacgttctctcctctccctatagtgagtcgtatta

SEQ ID NO: 3 - DNA, 77 bp, Artificial
  clone of aptamer PB.97.126.F_43-H1
gggagaggagagaacgttctcgaaatgatgcatgttcgtaaaatggcagtattggatcgttacaactagcatcgatg

SEQ ID NO: 4 - DNA, 76 bp, Artificial
  clone of aptamer PB.97.126.F_43-A2
gggagaggagagaacgttctcgtgccgaggtccggaaccttgatgattggcgggatcgttacgactagcatcgatg

SEQ ID NO: 5 - DNA, 76 bp, Artificial
  clone of aptamer PB.97.126.F_48-A1
gggagaggagagaacgttctcgcatttgggctagttgtgaaatggcagtattggatcgttacgactagcatcgatg

SEQ ID NO: 6 - DNA, 76 bp, Artificial
  clone of aptamer PB.97.126.F_48-B1
gggagaggagagaacgttctcgaatcgtagatagtcgtgaaatggcagtattggatcgttacgactagcatcgatg

SEQ ID NO: 7 - DNA, 76 bp, Artificial
  clone of aptamer PB.97.126.F_48-C1
gggagaggagagaacgttctcgttctagtcggtacgatatgttgacgaatccggatcgttacgactagcatcgatg

SEQ ID NO: 8 - DNA, 78 bp, Artificial
  clone of aptamer PB.97.126.F_48-D1
gggagaggagagaacgttctcgtttgatgaggcggacataatccgtgccgagcgggatcgttacgactagcatcgatg

SEQ ID NO: 9 - DNA, 77 bp, Artificial
  clone of aptamer PB.97.126.F_48-E1
gggagaggagagaacgttctcgaaggaaaagagtttagtattggccgtccgtgggatcgttacgactagcatcgatg

SEQ ID NO: 10 - DNA, 76 bp, Artificial; identical to SEQ ID NO: 4
  clone of aptamer PB.97.126.F_48-F1

SEQ ID NO: 11 - DNA, 76 bp, Artificial
  clone of aptamer PB.97.126.F_48-G1
gggagaggagagaacgttctcgtacggtccattgagtttgagatgtcgccatggatcgttacgactagcatcgatg

SEQ ID NO: 12 - DNA, 77 bp, Artificial
  clone of aptamer PB.97.126.F_48-B2
gggagaggagagaacgttctcgagttagtggtaactgatatgttgaattgtccggatcgttacgactagcatcgatg

SEQ ID NO: 13 - DNA, 76 bp, Artificial
  clone of aptamer PB.97.126.F_48-C2
gggagaggagagaacgttctcgcacggatggcgagaacagagattgctaggtggatcgttacgactagcatcgatg

SEQ ID NO: 14 - DNA, 76 bp, Artificial
  clone of aptamer PB.97.126.F_48-D2
gggagaggagagaacgttctcgntancgntncgccntgctaacgcntanttgggatcgttacgactagcatcgatg

SEQ ID NO: 15 - DNA, 77 bp, Artificial
  clone of aptamer PB.97.126.F_48-E2
gggagaggagagaacgttctcgaagatgagttttgtcgtgaaatggcagtattggatcgttacgactagcatcgatg

SEQ ID NO: 16 - DNA, 76 bp, Artificial
  clone of aptamer PB.97.126.F_48-F2
gggagaggagagaacgttctcgggatgccggattgatttctgatgggtactgggatcgttacgactagcatcgatg

SEQ ID NO: 17 - DNA, 76 bp, Artificial
  clone of aptamer PB.97.126.F_48-G2
gggagaggagagaacgttctcgaatggaatgcatgtccatcgctagcatttgggatcgttacgactagcatcgatg

SEQ ID NO: 18 - DNA, 76 bp, Artificial
  clone of aptamer PB.97.126.F_48-H2
gggagaggagagaacgttctcgtgctgaggtccggaaccttgatgattggcgggatcgttncnactagcatcgatg

SEQ ID NO: 19 - DNA, 76 bp, Artificial
  clone of aptamer PB.97.126.F_48-A3
gggagaggagagaacgttctcgctaattgctgagtcgtgaagtggcagtattggatcgttacgactagcatcgatg

SEQ ID NO: 20 - DNA, 76 bp, Artificial
  clone of aptamer PB.97.126.F_48-B3
gggagaggagagaacgttctcgtaacgatgtccggggcgaaaggctagcatgggatcgttacgactagcatcgatg

SEQ ID NO: 21 - DNA, 77 bp, Artificial
  clone of aptamer PB.97.126.F_48-C3
gggagaggagagaacgttctcgatgcgattgtcgagatttgtaagatagctgtggatcgttacgactagcatcgatg

SEQ ID NO: 22 - DNA, 76 bp, Artificial
  clone of aptamer PB.97.126.G_43-D3
gggagaggagagaacgttctcgcagaaaacatctttgcggttgaatacatgtggatcgttacgactagcatcgatg

SEQ ID NO: 23 - DNA, 76 bp, Artificial
  clone of aptamer PB.97.126.G_43-G3
gggagaggagagaacgttctcgaaaaaaganancnnccttcngaatacatgcggatcgttacgactagcatcgatg

SEQ ID NO: 24 - DNA, 76 bp, Artificial
  clone of aptamer PB.97.126.G_48-E3
gggagaggagagaacgttctcgagagtgattcgatgcttcangaatacatgtggatcgttacgactagcatcgatg

SEQ ID NO: 25 - DNA, 81 bp, Artificial
  clone of aptamer PB.97.126.G_48-F3
gggagaggagagaacgttctcgacannncntngctnggttgantacatgtgnntntcnnnancnntnntctntnanagggg

SEQ ID NO: 26 - DNA, 76 bp, Artificial
  clone of aptamer PB.97.126.G_48-H3
gggagaggagagaacgttctcgaagaaggaaagctgcaagtcgaatacacgcggatcgttacgactagcatcgatg

SEQ ID NO: 27 - DNA, 76 bp, Artificial
  clone of aptamer PB.97.126.G_48-A4
gggagaggagagaacgttctcgcaaaaacatcgattacagttgagtacatgtggatcgttacgactagcatcgatg

SEQ ID NO: 28 - DNA, 73 bp, Artificial
  clone of aptamer PB.97.126.G_48-B4
gggagaggagagaacgttctcgagacatcattgctcgttgaatacatgtggatcgttacgactagcatcgatg

SEQ ID NO: 29 - DNA, 76 bp, Artificial
  clone of aptamer PB.97.126.G_48-C4
gggagaggagagaacgttctcgccaaagtagcttcgacagtcgaatacatgtggatcgttacgactagcatcgatg

SEQ ID NO: 30 - DNA, 76 bp, Artificial
  clone of aptamer PB.97.126.G_48-D4
gggagaggagagaacgttctcgaaaatcagtactgtgcagtcgaatacatgcggatcgttacgactagcatcgatg

SEQ ID NO: 31 - DNA, 76 bp, Artificial
  clone of aptamer PB.97.126.G_48-E4
gggagaggagagaacgttctcgtaatgacatcaatgcttcttgaatacaggtggatcgttacgactagcatcgatg

SEQ ID NO: 32 - DNA, 75 bp, Artificial
  clone of aptamer PB.97.126.G_48-F4
gggagaggagagaacgttctcgagaaaaacgatctgtgacgtgtaatccgcggatcgttacgactagcatcgatg

SEQ ID NO: 33 - DNA, 76 bp, Artificial
  clone of aptamer PB.97.126.G_48-G4
gggagaggagagaacgttctcgcaacaaacgtcgacgcttctgaatacatgtggatcgttacgactagcatcgatg

SEQ ID NO: 34 - DNA, 76 bp, Artificial
  clone of aptamer PB.97.126.G_48-H4
gggagaggagagaacgttctcgtgatcatagaaatgctagctgaatacatgtggatcgttacgactagcatcgatg

SEQ ID NO: 35 - DNA, 75 bp, Artificial
  clone of aptamer PB.97.126.G_48-A5
gggagaggagagaacgttctcgcagcgtaaaatgcttttcgaagtacatgtggatcgttacgactagcatcgatg

SEQ ID NO: 36 - DNA, 76 bp, Artificial
  clone of aptamer PB.97.126.G_48-B5
gggagaggagagaacgttctcgccaagaatcaatcgcttgtcgaatacatgcggatcgttacgactagcatcgatg

SEQ ID NO: 37 - DNA, 76 bp, Artificial
  clone of aptamer PB.97.126.G_48-C5
gggagaggagagaacgttctcgtgatcatagaaatgctagctgagtacatgtggatcgttacgactagcatcgatg

SEQ ID NO: 38 - DNA, 76 bp, Artificial; identical to SEQ ID NO: 22
  clone of aptamer PB.97.126.G_48-D5

SEQ ID NO: 39 - DNA, 78 bp, Artificial
  clone of aptamer PB.97.126.G_48-E5
gggagaggagagaacgttctcgnaaacanncatctattgnagttgaatacatgtggatcgttacgactagcatcgatg

SEQ ID NO: 40 - DNA, 76 bp, Artificial
  clone of aptamer PB.97.126.G_48-F5
gggagaggagagaacgttctcgctaaagattcgctgcttgccgaatacatgtggatcgttacgactagcatcgatg

SEQ ID NO: 41 - DNA, 76 bp, Artificial
  clone of aptamer PB.97.126.H_43-H6
gggagaggagagaacgttctcgggttttgtctgcgtttgtgcgttgaacccgggatcgttacgactagcatcgatg

SEQ ID NO: 42 - DNA, 77 bp, Artificial
  clone of aptamer PB.97.126.H_43-F7
gggagaggagagaacgttctcgtgattacgtgatgaggatccgcgttttctcgggatcgttacgactagcatcgatg

SEQ ID NO: 43 - DNA, 76 bp, Artificial
  clone of aptamer PB.97.126.H_43-H7
gggagaggagagaacgttctcgttagtgaaaacgatcatgcatgtggatcgcggatcgttacgactagcatcgatg

SEQ ID NO: 44 - DNA, 75 bp, Artificial
  clone of aptamer PB.97.126.H_48-H5
gggagaggagagaacgttctcgtgttcattcgtttgcttatcgttgcatgtggatcgttacgactagcatcgatg

SEQ ID NO: 45 - DNA, 76 bp, Artificial
  clone of aptamer PB.97.126.H_48-A6
aggagaggagagaacgttctcggcagagtgtgatgtgcatccgcacgtgccgggatcgttacgactagcatcgatg

SEQ ID NO: 46 - DNA, 76 bp, Artificial
  clone of aptamer PB.97.126.H_48-B6
gggagaggagagaacgttctcgttagtaaatacgatcgtgcatgtggatcgcggatcgttacgactagcatcgatg

SEQ ID NO: 47 - DNA, 77 bp, Artificial
  clone of aptamer PB.97.126.H_48-C6
gggagaggagagaacgcccccctgattncgtgaagaggatccgcantttcncgggatcgttacgactagcatcgatg

SEQ ID NO: 48 - DNA, 76 bp, Artificial
  clone of aptamer PB.97.126.H_48-D6
gggagaggagagaacgttctcgtggctttggaacgggtacggatttggcacgggatcgttacgactagcatcgatg

SEQ ID NO: 49 - DNA, 77 bp, Artificial; identical to SEQ ID NO: 42
  clone of aptamer PB.97.126.H_48-E6

SEQ ID NO: 50 - DNA, 76 bp, Artificial
  clone of aptamer PB.97.126.H_48-F6
gggagaggagagaacgttctcgtcattggtgacngcgttgcatgtggatcgcggatcgttacgactagcatcgatg

SEQ ID NO: 51 - DNA, 76 bp, Artificial
  clone of aptamer PB.97.126.H_48-G6
gggagaggagagaacgttctcgntggtnnaangcttttgtngggntanntgtggatcgttacgactagcatcgatg

SEQ ID NO: 52 - DNA, 76 bp, Artificial
  clone of aptamer PB.97.126.H_48-A7
gggagaggagagaacgttctcgtggctttggaacgaattcggatttggcacgggatcgttacgactagcatcgatg

SEQ ID NO: 53 - DNA, 75 bp, Artificial
  clone of aptamer PB.97.126.H_48-B7
gggagaggagagaacgttctcgtgcgatgtcgtggatttccgtttcgcaagggatcgttacgactagcatcgatg

SEQ ID NO: 54 - DNA, 76 bp, Artificial
  clone of aptamer PB.97.126.H_48-C7
gggagaggagagaacgttctcgtgaagcagatgtcgttggcgacttagagggggatcgttacgactagcatcgatg

SEQ ID NO: 55 - DNA, 77 bp, Artificial
  clone of aptamer PB.97.126.H_48-D7
gggagaggagagaacgttctcgtgatttcgtgatgaggatccgcgttttctcgggatcgttacgactagcatcgatg

SEQ ID NO: 56 - DNA, 75 bp, Artificial
  clone of aptamer PB.97.126.H_48-E7
gggagaggagagaacgttctcgctagtaacgatgacttgatgagcatccgaggatcgttacgactagcatcgatg

SEQ ID NO: 57 - DNA, 76 bp, Artificial
  clone of aptamer PB.97.126.H_48-G7
gggagaggagagaacgttctcgtcataagtaacgacgttgcatgtggatcgcggatcgttacgactagcatcgatg

SEQ ID NO: 58 - DNA, 76 bp, Artificial
  clone of aptamer PB.97.126.H_48-A8
gggagaggagagaacgttctcgcaaggagatggttgctagctgagtacatgtggatcgttacgactagcatcgatg

SEQ ID NO: 59 - DNA, 78 bp, Artificial
  clone of aptamer PB.97.126.I_43-B8
gggagaggagagaacgttctcgcgatatgcagtttgagaagtcgcgcattcgggggatcgttacgactagcatcgatg

SEQ ID NO: 60 - DNA, 75 bp, Artificial
  clone of aptamer PB.97.126.I_48-C8
gggagaggagagaacgttctcgtgcgacgggcttcttgtgtcattcgcatgggatcgttacgactagcatcgatg

SEQ ID NO: 61 - DNA, 76 bp, Artificial
  clone of aptamer PB.97.126.I_48-D8
gggagaggagagaacgttctcggcattgcagttgataggtcgcgcagtgctgggatcgttacgactagcatcgatg

SEQ ID NO: 62 - DNA, 78 bp, Artificial
  clone of aptamer PB.97.126.I_48-E8
gggagaggagagaacgttctcgcgatatgcagtctgagaagtcgcgcattcgagggatcgttacgactagcatcgatg

SEQ ID NO: 63 - DNA, 76 bp, Artificial
  clone of aptamer PB.97.126.I_48-F8
gggagaggagagaacgttctcgtgtagcaagcatgtggatcgcgactgcacgggatcgttacgactagcatcgatg

SEQ ID NO: 64 - DNA, 76 bp, Artificial
  clone of aptamer PB.97.126.I_48-G8
gggagaggagagaacgttctcggataagcagttgagatgtcgcgctttgacgggatcgttacgactagcatcgatg

SEQ ID NO: 65 - DNA, 75 bp, Artificial
  clone of aptamer PB.97.126.I_48-H8
gggagaggagagaacgttctcgatgancantttgagaagtcgcgcttgtcgggatcgttacgactagcatcgatg

SEQ ID NO: 66 - DNA, 75 bp, Artificial
  clone of aptamer PB.97.126.I_48-A9
gggagaggagagaacgttctcgagtaatgcagtggaagtcgcgcattacctgggatcgttacgactagcatcatg

SEQ ID NO: 67 - DNA, 78 bp, Artificial; identical to SEQ ID NO: 59
  clone of aptamer PB.97.126.I_48-B9

SEQ ID NO: 68 - DNA, 73 bp, Artificial
  clone of aptamer PB.97.126.I_48-C9
gggagaggagagaacgttctcgtgatncagttganaagtcncgcatacaggatcgttacgactagcatcgatg

SEQ ID NO: 69 - DNA, 76 bp, Artificial
  clone of aptamer PB.97.126.I_48-D9
gggagaggagagaacgttctcgagtaatgctgtggaagtcgcgcatttcctgggatcgttacgactagcatcgatg

SEQ ID NO: 70 - DNA, 76 bp, Artificial; identical to SEQ ID NO: 61
  clone of aptamer PB.97.126.I_48-D8

SEQ ID NO: 71 - DNA, 78 bp, Artificial
  clone of aptamer PB.97.126.I_48-F9
gggagaggagagaacgttctcgcgatatgcagtttgggaagtcgcgcattcgagggatcgttacgactagcatcgatg

SEQ ID NO: 72 - DNA, 78 bp, Artificial
  clone of aptamer PB.97.126.I_48-G9
gggagaggagagaacgttctcgcnatatgctgtttganaantcgcgcattcgggggatcgttacgactagcatcgatg

SEQ ID NO: 73 - DNA, 78 bp, Artificial
  clone of aptamer PB.97.126.I_48-H9
gggagaggagagaacgttctcgcgtagattgggctgaatgggatatctttagcgggatcgttacgactagcatcgatg

SEQ ID NO: 74 - DNA, 78 bp, Artificial
  clone of aptamer PB.97.126.I_48-B10
gggagaggagagaacgttctcgcgatatgcagtttgagaagtcgcgctttcgagggatcgttacgactagcatcgatg

SEQ ID NO: 75 - DNA, 78 bp, Artificial
  clone of aptamer PB.97.126.I_48-D10
gggagaggagagaacgttctcgtcaatctgatgtagcctcacgtgggcggagtcggatcgttacgactagcatcgatg

SEQ ID NO: 76 - DNA, 45 bp, Artificial
  clone of aptamer PB.97.126.J_48-F10
gggagaggagagaacgttctcggatcgttacgactagcatcgatg

SEQ ID NO: 77 - DNA, 45 bp, Artificial; identical to SEQ ID NO: 76
  clone of aptamer PB.97.126.J_48-G10

SEQ ID NO: 78 - DNA, 76 bp, Artificial
  clone of aptamer PB.97.126.J_48-H10
gggagaggagagaacgttctcggtggtgttgctgaactgtcgcgtttcgccgggatcgttacgactagcatcgatg

SEQ ID NO: 79 - DNA, 77 bp, Artificial
  clone of aptamer PB.97.126.J_48-A11
gggagaggagagaacgttctcgtcgcgattgcatattttccgccttgctgtgaggatcgttacgactagcatcgatg

SEQ ID NO: 80 - DNA, 78 bp, Artificial
  clone of aptamer PB.97.126.J_48-B11
gggagaggagagaacgttctcgcgatttgcagtttgagatgtcgcgcattcgagggatcgttacgactagcatcgatg

SEQ ID NO: 81 - DNA, 78 bp, Artificial; identical to SEQ ID NO: 59
  clone of aptamer PB.97.126.J_48-C11

SEQ ID NO: 82 - DNA, 76 bp, Artificial
  clone of aptamer PB.97.126.J_48-D11
gggagaggagagaacgttctcgttggtgcagtttgagatgtcgcgcaccttgggatcgttacgactagcatcgatg

SEQ ID NO: 83 - DNA, 80 bp, Artificial
  clone of aptamer PB.97.126.J_48-E11
gggagaggagagaacgttctcggtattggttccattaagctggacactctgctccgggatcgttacgactagcatcgatg

SEQ ID NO: 84 - DNA, 76 bp, Artificial
  clone of aptamer PB.97.126.J_48-F11
gggagaggagagaacgttctcgttggtgcagtttgagatgtcgcgcgccttgggatcgttacgactagcatcgatg

SEQ ID NO: 85 - DNA, 78 bp, Artificial
  clone of aptamer PB.97.126.J_48-G11
gggagaggagagaacgttctcgcgatatgcagtttgagaagtcgcgcattcgagggatcgttacnactagcatcgatg

SEQ ID NO: 86 - DNA, 78 bp, Artificial; identical to SEQ ID NO: 59
  clone of aptamer PB.97.126.J_48-A12

SEQ ID NO: 87 - DNA, 80 bp, Artificial
  clone of aptamer PB.97.126.J_48-B12
gggagaggagagaacgctctcggggacnnaaanncgaattgncgcgtgngtccgggggagcgcccgactagtcatcgatg

SEQ ID NO: 88 - DNA, 78 bp, Artificial
  clone of aptamer PB.97.126.J_48-C12
gggagaggagagaacgttctcgcgatatgnantttgagaagtcgcgcattcgggggatcgttacgactagcatcgatg

SEQ ID NO: 89 - DNA, 75 bp, Artificial
  clone of aptamer PB.97.126.J_48-D12
gggagaggagagaacgttctcggtgtacagcttgagatgtcgcgtactccgggatcgttacgactagcatcgatg

SEQ ID NO: 90 - DNA, 78 bp, Artificial; identical to SEQ ID NO: 59
  clone of aptamer PB.97.126.J_48-E12

SEQ ID NO: 91 - DNA, 76 bp, Artificial
  clone of aptamer PB.97.126.J_48-F12
gggagaggagagaacgttctcgagtaagaaagctgaatggtcgcacttctcgggatcgttacgactagcatcgatg

SEQ ID NO: 92 - DNA, 78 bp, Artificial
  clone of aptamer PB.97.126.J_48-G12
agggagaggaagaacgttctcgcgatgtgcagtttgagaagtcgcgcattcgagggatcgttacgactagcatcgatg

SEQ ID NO: 93 - DNA, 76 bp, Artificial
  clone of aptamer PB.97.126.J_48-H12
gggagaggagagaacgttctcgaaagaatcagcatgcggatcgcggctttcgggatcgttacgactagcatcgatg

SEQ ID NO: 94 - DNA, 79 bp, Artificial
  clone of aptamer PB.97.126.A_44-A1
gggagaggagagaacgttctcgantccantntncntggaggagtaagtacctgagggatcgttacgactagcatcgatg

SEQ ID NO: 95 - DNA, 76 bp, Artificial
  clone of aptamer PB.97.126.A_44-B1
gggagaggagagaacgttctcgggaaacaaggaacttagagttanttgaccgggatcgttacgactagcatcgatg

SEQ ID NO: 96 - DNA, 76 bp, Artificial
  clone of aptamer PB.97.126.A_44-C1
gggagaggagagaacgttctcgtaccatgcaaggaacataatagttagcgtgggatcgttacgactagcatcgatg

SEQ ID NO: 97 - DNA, 76 bp, Artificial
  clone of aptamer PB.97.126.A_44-D1
gggagaggagagaacgttctcgggacacaaggaacacaatagttagtgtacgggatcgttacgactagcatcgatg

SEQ ID NO: 98 - DNA, 76 bp, Artificial
  clone of aptamer PB.97.126.A_44-E1
gggagaggagagaacgttctcgtctgcaaggaacacaatagttagcattgcgggatcgttacgactagcatcgatg

SEQ ID NO: 99 - DNA, 76 bp, Artificial
  clone of aptamer PB.97.126.A_44-F1
gggagaggagagaacgttctcgcgccaacaaagctggagtacttagagcgcgggatcgttacgactagcatcgatg

SEQ ID NO: 100 - DNA, 76 bp, Artificial
  clone of aptamer PB.97.126.A_44-G1
gggagaggagagaacgttctcgattgcaaaatagctgtagaactaagcaatcggatcgttacgactagcatcgatg

SEQ ID NO: 101 - DNA, 76 bp, Artificial
  clone of aptamer PB.97.126.A_44-H1
gggagaggagagaacgttctcgtgagatgactatgttaagatgacgctgttgggatcgttacgactagcatcgatg

SEQ ID NO: 102 - DNA, 76 bp, Artificial
  clone of aptamer PB.97.126.A_44-A2
gggagaggagagaacgttctcggganacaaggaacncaatatttagtgaacgggatcgttacgactagcatcgatg

SEQ ID NO: 103 - DNA, 76 bp, Artificial
  clone of aptamer PB.97.126.A_44-B2
gggagaggagagaacgttctcgccaaggaacacaatagttaggtgagaatcgggatcgttacgactagcatcgatg

SEQ ID NO: 104 - DNA, 75 bp, Artificial
  clone of aptamer PB.97.126.A_44-C2
gggagaggagagaacgttctcggtacaaggaacacaatagttagtgccgtgggatcgttacgactagcatcgatg

SEQ ID NO: 105 - DNA, 77 bp, Artificial
  clone of aptamer PB.97.126.A_44-D2
gggagaggagagaacgttctcgattcaacggtccaaaaaagctgtagtacttaggatcgttacgactagcatcgatg

SEQ ID NO: 106 - DNA, 76 bp, Artificial
  clone of aptamer PB.97.126.A_44-E2
gggagaggagagaacgttctcgcaatgcaaggaacacaatagttagcagccgggatcgttacgactagcatcgatg

SEQ ID NO: 107 - DNA, 76 bp, Artificial
  clone of aptamer PB.97.126.A_44-F2
gggagaggagagaacgttctcgaaaggagaaagctgaagtacttactatgcgggatcgttacgactagcatcgatg

SEQ ID NO: 108 - DNA, 76 bp, Artificial
  clone of aptamer PB.97.126.A_44-G2
gggagaggagagaacgttctcgcacaaggaacacaatagttagtgcaagacgggatcgttacgactagcatcgatg

SEQ ID NO: 109 - DNA, 76 bp, Artificial
  clone of aptamer PB.97.126.A_44-A3
gggagaggagagaacgttctcgcacaaggaactacgagttagtgtgggagtgggatcgttacgactagcatcgatg

SEQ ID NO: 110 - DNA, 76 bp, Artificial
  clone of aptamer PB.97.126.A_44-B3
gggagaggagagaacgttctcgcacaaggaacacaatagttagtgcaagacgggatcgttacgactagcatcgata

SEQ ID NO: 111 - DNA, 75 bp, Artificial
  clone of aptamer PB.97.126.A_44-C3
gggagaggagagaacgttctcggcgggaaaatagctgtagtactaacccacggatcgttacgactagcatcgatg

SEQ ID NO: 112 - DNA, 76 bp, Artificial
  clone of aptamer PB.97.126.B_44-E3
gggagaggagagaacgttctcggcctcaaggaaaagaaaatttagaggcccgggatcgttacgactagcatcgatg

SEQ ID NO: 113 - DNA, 76 bp, Artificial
  clone of aptamer PB.97.126.B_44-F3
gggagaggagagaacgttctcggaacaagatagctgaaggactaagtttacgggatcgttacgactagcatcgatg

SEQ ID NO: 114 - DNA, 76 bp, Artificial; identical to SEQ ID NO: 113
  clone of aptamer PB.97.126.B_44-G3

SEQ ID NO: 115 - DNA, 77 bp, Artificial
  clone of aptamer PB.97.126.B_44-H3
gggagaggagagaacgttctcggagccaaggaaacgaagatttaggctcattgggatcgttacgactagcatcgatg

SEQ ID NO: 116 - DNA, 76 bp, Artificial
  clone of aptamer PB.97.126.B_44-A4
gggagaggagagaacgttctcgatcacaagaaatgtggganggtagtgatncnnntcgttncgactagcatcgatg

SEQ ID NO: 117 - DNA, 76 bp, Artificial
  clone of aptamer PB.97.126.B_44-B4
gggagaggagagaacgttctcgtcgaaagggagctttgtctcgggacagaacggatcgttacgactagcatcgatg

SEQ ID NO: 118 - DNA, 76 bp, Artificial
  clone of aptamer PB.97.126.B_44-C4
gggagaggagagaacgntctcgtgcaaagatagctggaggactaatgcggcgggatcgttacgactagcatcgatg

SEQ ID NO: 119 - DNA, 76 bp, Artificial; identical to SEQ ID NO: 117
  clone of aptamer PB.97.126.B_44-D4

SEQ ID NO: 120 - DNA, 76 bp, Artificial
  clone of aptamer PB.97.126.B_44-E4
gggagaggagagaacgttctcgncnaaggngagctttgtcccnggacanaangnatcgttacaactagcatcgatg

SEQ ID NO: 121 - DNA, 76 bp, Artificial; identical to SEQ ID NO: 113
  clone of aptamer PB.97.126.B_44-F4

SEQ ID NO: 122 - DNA, 76 bp, Artificial; identical to SEQ ID NO: 113
  clone of aptamer PB.97.126.B_44-G4

SEQ ID NO: 123 - DNA, 78 bp, Artificial
  clone of aptamer PB.97.126.B_44-H4
gggagaggagagaacgttctcggcgcaaaaaaagctggagtacttagtgtcgagggatcgttacgactagcatcgatg

SEQ ID NO: 124 - DNA, 76 bp, Artificial; identical to SEQ ID NO: 117
  clone of aptamer PB.97.126.B_44-A5

SEQ ID NO: 125 - DNA, 76 bp, Artificial
  clone of aptamer PB.97.126.B_44-B5
gggagaggagagaacgttctcgacacaagaaagctgcagaacttagggtcgtggatcgttacgactagcatcgatg

SEQ ID NO: 126 - DNA, 76 bp, Artificial
  clone of aptamer PB.97.126.B_44-C5
gggagaggagagaacgttctcggaacnggattgttgaaggactaantttacgggatcgttacgactagcatcgatg

SEQ ID NO: 127 - DNA, 76 bp, Artificial
  clone of aptamer PB.97.126.B_44-D5
gggagaggagagaacgttctcggcctcaagggaaagaaaatttagaggcccgggatcgttacgactagcatcgatg

SEQ ID NO: 128 - DNA, 77 bp, Artificial
  clone of aptamer PB.97.126.B_44-E5
gggagaggagagaacgttctcggaaacaagcttagaaattcgcacccttgccgggatcgttacgactagcatcgatg

SEQ ID NO: 129 - DNA, 75 bp, Artificial
  clone of aptamer PB.97.126.B_44-F5
gggagaggagagaacgttctcgaaagaaaaaagctggagaacttacttccgggatcgttacgactagcatcgatg

SEQ ID NO: 130 - DNA, 78 bp, Artificial
  clone of aptamer PB.97.126.B_44-G5
gggagaggagagaacgttctcggtgattgtactcacatagaaatggcaacactgggatcgttacgactagcatcgatg

SEQ ID NO: 131 - DNA, 76 bp, Artificial
  clone of aptamer PB.97.126.C_44-H5
gggagaggagagaacgttctcgggttcaaggaacatgatagttagaacccgcggatcgttacgactagcatcgatg

SEQ ID NO: 132 - DNA, 77 bp, Artificial
  clone of aptamer PB.97.126.C_44-A6
gggagaggagagaacgttctcgttccgaaaggaacacaatagttatcggattgggatcgttacgactagcatcgatg

SEQ ID NO: 133 - DNA, 76 bp, Artificial; identical to SEQ ID NO: 98
  clone of aptamer PB.97.126.C_44-B6

SEQ ID NO: 134 - DNA, 74 bp, Artificial
  clone of aptamer PB.97.126.C_44-C6
gggagaggagagaacgttctcggtacaaggaacacaatagttagtgccggggatcgttacgactagcatcgatg

SEQ ID NO: 135 - DNA, 75 bp, Artificial
  clone of aptamer PB.97.126.C_44-D6
gggagaggagagaacgttctcggaactcagagatcctatgtggaccagagaggatcgttacgactagcatcgatg

SEQ ID NO: 136 - DNA, 76 bp, Artificial
  clone of aptamer PB.97.126.C_44-E6
gggagaggagagaacgttctcgctgagcaaggaacgtaatagttagcctgcgggatcgttacgactagcatcgatg

SEQ ID NO: 137 - DNA, 77 bp, Artificial
  clone of aptamer PB.97.126.C_44-F6
gggagaggagagaacgttctcgnannnataaatgatggatcncttattgtnnaggatcgttacgactagcatcgatg

SEQ ID NO: 138 - DNA, 74 bp, Artificial
  clone of aptamer PB.97.126.C_44-G6
gggagaggagagaacgttctcggcttggaaaaatagcttttgggcatccgggatcgttacgactagcatcgatg

SEQ ID NO: 139 - DNA, 76 bp, Artificial
  clone of aptamer PB.97.126.C_44-H6
gggagaggagagaacgttctcgggttcaaggaacatgatagctagaacccgcggatcgttacgactagcatcgatg

SEQ ID NO: 140 - DNA, 76 bp, Artificial; identical to SEQ ID NO: 131
  clone of aptamer PB.97.126.C_44-A7

SEQ ID NO: 141 - DNA, 76 bp, Artificial
  clone of aptamer PB.97.126.C_44-B7
gggagaggagagaacgttctcgtgggcagggaacacaatagttagcctacgcggatcgttacgactagcatcgatg

SEQ ID NO: 142 - DNA, 75 bp, Artificial
  clone of aptamer PB.97.126.C_44-C7
gggagaggagagaacgttctcgcgtgaaaggaacacaatagttatcgtgcgggatcgttacgactagcatcgatg

SEQ ID NO: 143 - DNA, 77 bp, Artificial
  clone of aptamer PB.97.126.C_44-D7
gggagaggagagaacgttctcgcgaggtttatcctagacgactaaccgcctggggatcgttacgactagcatcgatg

SEQ ID NO: 144 - DNA, 76 bp, Artificial
  clone of aptamer PB.97.126.C_44-F7
gggagaggagagaacgttctcgtctgctaggaacacaatagttagcattgcgggatcgttacgactagcatcgatg

SEQ ID NO: 145 - DNA, 77 bp, Artificial
  clone of aptamer PB.97.126.C_44-G7
gggagaggagagaacgttctcgcacaaggaactacgagttagtgtgggagtggggatcgttacgactagcatcgatg

SEQ ID NO: 146 - DNA, 77 bp, Artificial
  clone of aptamer PB.97.126.C_44-H7
gggagaggagagaacgttctcgtgacacgaggaacttagagttagtagcacgaggatcgttacgactagcatcgatg

SEQ ID NO: 147 - DNA, 76 bp, Artificial
  clone of aptamer PB.97.126.C_44-A8
gggagaggagagaacgttctcggcggcgaaggaacacaatagttacgtcccgggatcgttacgactagcatcgatg

SEQ ID NO: 148 - DNA, 76 bp, Artificial
  clone of aptamer PB.97.126.C_44-B8
gggagaggagagaacgttctcgagcccaaaaaagctgaagtactttgggcagggatcgttacgactagcatcgatg

SEQ ID NO: 149 - DNA, 75 bp, Artificial; identical to SEQ ID NO: 104
  clone of aptamer PB.97.126.D_44-D8

SEQ ID NO: 150 - DNA, 45 bp, Artificial; identical to SEQ ID NO: 76
  clone of aptamer PB.97.126.D_44-E8

SEQ ID NO: 151 - DNA, 76 bp, Artificial
  clone of aptamer PB.97.126.D_44-G8
gggagaggagagaacgttctcgtgcgcaaggaacacaatagttagggcgcgaggatcgttacgactagcattgatg

SEQ ID NO: 152 - DNA, 76 bp, Artificial
  clone of aptamer PB.97.126.D_44-H8
gggagaggagagaacgttctcggaatggaaggaacacaatagttaccagacgggatcgttacgactagcatcgatg

SEQ ID NO: 153 - DNA, 76 bp, Artificial; identical to SEQ ID NO: 98
  clone of aptamer PB.97.126.D_44-A9

SEQ ID NO: 154 - DNA, 76 bp, Artificial
  clone of aptamer PB.97.126.D_44-B9
gggagaggagagaacgttctcgagacaagacagctggaggactaagtcacgaggatcgttacgactagcatcgatg

SEQ ID NO: 155 - DNA, 76 bp, Artificial
  clone of aptamer PB.97.126.D_44-C9
gggagaggagagaacgttctcgatgcccgcaaaggaacacgatagttatgcgggatcgttacgactagcatcgatg

SEQ ID NO: 156 - DNA, 76 bp, Artificial
  clone of aptamer PB.97.126.D_44-D9
gggagaggagagaacgttctcgtctgnnaggaacacaatatttagcattgcgggatcgttacgactagcatcgatg

SEQ ID NO: 157 - DNA, 76 bp, Artificial
  clone of aptamer PB.97.126.D_44-E9
gggagaggagagaacgttctcgaatgtgcggagcagtattggtacactttcgggatcgttacgactagcatcgatg

SEQ ID NO: 158 - DNA, 76 bp, Artificial; identical to SEQ ID NO: 103
  clone of aptamer PB.97.126.D_44-F9

SEQ ID NO: 159 - DNA, 76 bp, Artificial; identical to SEQ ID NO: 103
  clone of aptamer PB.97.126.D_44-G9

SEQ ID NO: 160 - DNA, 76 bp, Artificial
  clone of aptamer PB.97.126.D_44-H9
gggagaggagagaacgttctcgggaagcaaggaacttagagttagttgaccgggatcgttacgactagcatcgatg

SEQ ID NO: 161 - DNA, 76 bp, Artificial
  clone of aptamer PB.97.126.D_44-A10
gggagaggagagaacgttctcgtgggcaaggaacacaatagttagcctacgcggatcgttacgactagcatcgatg

SEQ ID NO: 162 - DNA, 76 bp, Artificial
  clone of aptamer PB.97.126.D_44-B10
gggagaggagagaacgttctcgtcgggcatggaacacaatagttagaccgcgggatcgttacgactagcatcgatg

SEQ ID NO: 163 - DNA, 75 bp, Artificial
  clone of aptamer PB.97.126.D_44-C10
gggagaggagagaacgttctcggtcgcaaggaacataatagttagcggaggggatcgttacgactagcatcgatg

SEQ ID NO: 164 - DNA, 76 bp, Artificial; identical to SEQ ID NO: 98
  clone of aptamer PB.97.126.D_44-D10

SEQ ID NO: 165 - DNA, 77 bp, Artificial
  clone of aptamer PB.97.126.D_44-E10
gggagaggagagaacgttctcgccgacaatcagctcggatcgtgtgctacgctggatcgttacgactagcatcgatg

SEQ ID NO: 166 - DNA, 77 bp, Artificial
  clone of aptamer PB.97.126.E_44-F10
gggagaggagagaacgttctcgagacaagatagctgaaggactaagtcacgagggatcgttacgactagcatcgatg

SEQ ID NO: 167 - DNA, 76 bp, Artificial
  clone of aptamer PB.97.126.E_44-G10
gggagaggagagaacgttctcggaacaagatagctgaaggactaagtttgcgggatcgttacgactagcatcgatg

SEQ ID NO: 168 - DNA, 77 bp, Artificial
  clone of aptamer PB.97.126.E_44-H10
gggagaggagagaacgttctcggagncaaggaaacnaatatttaggctcantggnnncnttncanctagcnncnnta

SEQ ID NO: 169 - DNA, 76 bp, Artificial; identical to SEQ ID NO: 98
  clone of aptamer PB.97.126.E_44-A11

SEQ ID NO: 170 - DNA, 76 bp, Artificial; identical to SEQ ID NO: 113
  clone of aptamer PB.97.126.E_44-B11

SEQ ID NO: 171 - DNA, 45 bp, Artificial; identical to SEQ ID NO: 76
  clone of aptamer PB.97.126.E_44-C11

SEQ ID NO: 172 - DNA, 78 bp, Artificial
  clone of aptamer PB.97.126.E_44-D11
gggagaggagagaacgttctcggtgatagtactcacatagaaatggctacactgggatcgttacgactagcatcgatg

SEQ ID NO: 173 - DNA, 76 bp, Artificial
  clone of aptamer PB.97.126.E_44-E11
gggagaggagagaacgttctcgcctgggcaaggaacagaaaagttagcgccaggatcgttacgactagcatcgatg

SEQ ID NO: 174 - DNA, 76 bp, Artificial
  clone of aptamer PB.97.126.E_44-F11
gggagaggagagaacgttctcgtaacggacaaaaggaaccgggaagttatctggatcgttacgactagcatcgatg

SEQ ID NO: 175 - DNA, 76 bp, Artificial
  clone of aptamer PB.97.126.E_44-G11
gggagaggagagaacgttctcgcgcacaagatagagaagactaagtccgcggggatcgttacgactagcatcgatg

SEQ ID NO: 176 - DNA, 76 bp, Artificial
  clone of aptamer PB.97.126.E_44-H11
gggagaggagagaacgttctcgcgcacaagatagagaagactaagttcgcggggatcgttacgactagcatcgatg

SEQ ID NO: 177 - DNA, 76 bp, Artificial
  clone of aptamer PB.97.126.E_44-A12
gggagaggagagaacgttctcgcgccaataaagctggagtacttagagcgcgggatcgttacgactagcatcgatg

SEQ ID NO: 178 - DNA, 76 bp, Artificial
  clone of aptamer PB.97.126.E_44-B12
gggagaggagagaacgttctcgggaaacaaggaacttagagttagttgaccgggatcgttacgactagcatcgatg

SEQ ID NO: 179 - DNA, 76 bp, Artificial
  clone of aptamer PB.97.126.E_44-C12
gggagaggagagaacgttctcgctagcaagataggtgggactaagctagtgaggatcgttacgactagcatcgatg

SEQ ID NO: 180 - DNA, 76 bp, Artificial
  clone of aptamer PB.97.126.E_44-D12
gggagaggagagaacgttctcgtcgaaggggagctttgtctcgggacagaacggatcgttacgactagcatcgatg

SEQ ID NO: 181 - DNA, 76 bp, Artificial; identical to SEQ ID NO: 113
  clone of aptamer PB.97.126.E_44-E12

SEQ ID NO: 182 - DNA, 76 bp, Artificial; identical to SEQ ID NO: 167
  clone of aptamer PB.97.126.E_44-G12

SEQ ID NO: 183 - DNA, 77 bp, Artificial
  clone of aptamer PB.97.126.E_44-H12
gggagaggaganntccccncncggaaaaanaaaaaagaagaantangttngggggatcgttacgactagcatcgatg

SEQ ID NO: 184 - RNA, 30 nt, Artificial
  r/mGmH aptamer ARC224 -Stabilized VEGF Aptamer
cgnunugcnguuugngnngucgcgcnuucg

SEQ ID NO: 185 - RNA, 30 nt, Artificial; identical to SEQ ID NO: 184
  r/mGmH aptamer ARC225 - Stabilized VEGF Aptamer

SEQ ID NO: 186 - RNA, 24 nt, Artificial
  r/mGmH aptamer ARC226 Single-hydroxy VEGF aptamer
gnucnugcnuguggnucgcggnuc

SEQ ID NO: 187 - RNA, 23 nt, Artificial
  r/mGmH aptamer ARC245 VEGF Aptamer
nugcnguuugngnngucgcgcnu

SEQ ID NO: 188 - RNA, 23 nt, Artificial
  r/mGmH aptamer ARC259 hVEGF Aptamer
ncgcnguuugngnngucgcgcgu

SEQ ID NO: 189 - DNA, 82 bp, Artificial
  clone of aptamer PDGF-BB  ARX36.SCK.A12.M13F
gggagaggagagaacgttctactatgaagggttttaaagatgacacattagccgctgtcgatcgatcgatcgatgaagggcg

SEQ ID NO: 190 - DNA, 75 bp, Artificial
  clone of aptamer IgE A5
gggagaggagagaacgttctacaggcagttctggggacccatgggggaagtgcgctgtcgatcgatcgatcgatg

SEQ ID NO: 191 - DNA, 75 bp, Artificial
  clone of aptamer IgE A6
gggagaggagagaacgttctacgattagcagggagggagagtgcgaagaggacgctgtcgatcgatcgatcgatg

SEQ ID NO: 192 - DNA, 75 bp, Artificial
  clone of aptamer IgE A7
gggagaggagagaacgttctacactctggggacccgtgggggagtgcagcaacgctgtcgatcgatcgatcgatg

SEQ ID NO: 193 - DNA, 75 bp, Artificial
  clone of aptamer IgE A8
gggagaggagagaacgttctacaagcagttctggggacccatgggggaagtgcgctgtcgatcgatcgatcgatg

SEQ ID NO: 194 - DNA, 74 bp, Artificial
  clone of aptamer IgE B5
gggagaggagagaacgttctacgaggtgagggtctacaatggagggatggtcgctgtcgatcgatcgatcgatg

SEQ ID NO: 195 - DNA, 75 bp, Artificial
  clone of aptamer IgE B6
gggagaggagagaacgttctacccgcagcatagcctgnggacccatgnggggcgctgtcgatcgatcgatcgatg

SEQ ID NO: 196 - DNA, 75 bp, Artificial
  clone of aptamer IgE B7
gggagaggagagaacgttctactggggggcgtgttcattagcagcgtcgtgtcgctgtcgatcgatcgatcgatg

SEQ ID NO: 197 - DNA, 75 bp, Artificial; identical to SEQ ID NO: 190
  clone of aptamer IgE B8

SEQ ID NO: 198 - DNA, 75 bp, Artificial
  clone of aptamer IgE C5
gggagaggagagaacgttctacgcagcgcatctggggacccaagaggggattcgctgtcgatcgatcgatcgatg

SEQ ID NO: 199 - DNA, 75 bp, Artificial; identical to SEQ ID NO: 190
  clone of aptamer IgE C6

SEQ ID NO: 200 - DNA, 73 bp, Artificial
  clone of aptamer IgE C7
gggagaggagagaacgttctacgggatgggtagttggatggaaatgggaacgctgtcgatcgatcgatcgatg

SEQ ID NO: 201 - DNA, 74 bp, Artificial
  clone of aptamer IgE C8
gggagaggagagaacgttctacgaggtgtagggatagaggggtgtaggtaacgctgtcgatcgatcgatcgatg

SEQ ID NO: 202 - DNA, 75 bp, Artificial
  clone of aptamer IgE D5
gggagaggagagaacgttctacaggagtggagctacagagagggttaggggtcgctgtcgatcgatcgatcgatg

SEQ ID NO: 203 - DNA, 75 bp, Artificial
  clone of aptamer IgE D6
gggagaggagagaacgttctacggatgttgggagtgatagaaggaaggggagcgctgtcgatcgatcgatcgatg

SEQ ID NO: 204 - DNA, 75 bp, Artificial; identical to SEQ ID NO: 190
  clone of aptamer IgE D7

SEQ ID NO: 205 - DNA, 75 bp, Artificial; identical to SEQ ID NO: 190
  clone of aptamer IgE D8

SEQ ID NO: 206 - DNA, 75 bp, Artificial; identical to SEQ ID NO: 190
  clone of aptamer IgE E5

SEQ ID NO: 207 - DNA, 76 bp, Artificial
  clone of aptamer IgE E6
gggagaggagagaacgttctacttggggtggaaggagtaagggaggtgctgatcgctgtcgatcgatcgatcgatg

SEQ ID NO: 208 - DNA, 75 bp, Artificial
  clone of aptamer IgE E7
gggagaggagagaacgttctacgtattaggggggaaggggaggaatagatcacgctgtcgatcgatcgatcgatg

SEQ ID NO: 209 - DNA, 76 bp, Artificial
  clone of aptamer IgE E8
gggagaggagagaacgttctacagggagagagtgttgagtgaagaggaggagtcgctgtcgatcgatcgatcgatg

SEQ ID NO: 210 - DNA, 75 bp, Artificial
  clone of aptamer IgE F5
gggagaggagagaacgttctacattgtgctcctggggcccagtggggagccacgctgtcgatcgatcgatcgatg

SEQ ID NO: 211 - DNA, 75 bp, Artificial
  clone of aptamer IgE F6
gggagaggagagaacgttctacgagcagccctggggcccggagggggatggtcgctgtcgatcgatcgatcgatg

SEQ ID NO: 212 - DNA, 75 bp, Artificial; identical to SEQ ID NO: 190
  clone of aptamer IgE F7

SEQ ID NO: 213 - DNA, 75 bp, Artificial
  clone of aptamer IgE F8
gggagaggagagaacgttctaccaacggcatcctgggccccacaggggatgtcgctgtcgatcgatcgatcgatg

SEQ ID NO: 214 - DNA, 74 bp, Artificial
  clone of aptamer IgE G5
gggagaggagagaacgttctacgagtggatagggaagaaggggagtagtcacgctgtcgatcgatcgatcgatg

SEQ ID NO: 215 - DNA, 75 bp, Artificial
  clone of aptamer IgE G6
gggagaggagagaacgttctacccgcagcatagcctggggacccatggggggcgctgtcgatcgatcgatcgatg

SEQ ID NO: 216 - DNA, 76 bp, Artificial
  clone of aptamer IgE G7
gggagaggagagaacgttctacggtcgcgtgtgggggacggatgggtattggtcgctgtcnatcgatcgatcnatg

SEQ ID NO: 217 - DNA, 75 bp, Artificial; identical to SEQ ID NO: 215
  clone of aptamer IgE G8

SEQ ID NO: 218 - DNA, 75 bp, Artificial; identical to SEQ ID NO: 215
  clone of aptamer IgE H5

SEQ ID NO: 219 - DNA, 75 bp, Artificial
  clone of aptamer IgE H6
gggagaggagagaacgttctacggggttacgtcgcacgatacatgcattcatcgctgtcgatcgatcgatcgatg

SEQ ID NO: 220 - DNA, 75 bp, Artificial
  clone of aptamer IgE H7
gggagaggagagaacgttctactagcgaggaggggttttctatttttgcgatcgctgtcgatcgatcgatcgatg

SEQ ID NO: 221 - DNA, 75 bp, Artificial
  clone of aptamer Thrombin A1
gggagaggagagaacgttctacgtgtgatggggtgagaggatgagttagtgacgctgtcgatcgatcgatcgatg

SEQ ID NO: 222 - DNA, 74 bp, Artificial
  clone of aptamer Thrombin A2
gggagaggagagaacgttctacaatgggagggtaatagtgatgaggagaggcgctgtcgatcgatcgatcgatg

SEQ ID NO: 223 - DNA, 75 bp, Artificial
  clone of aptamer Thrombin A3
gggagaggagagaacgttctacgggtcgtgagataatggctcccgtattcagcgctgtcgatcgatcgatcgatg

SEQ ID NO: 224 - DNA, 75 bp, Artificial; identical to SEQ ID NO: 223
  clone of aptamer Thrombin A4

SEQ ID NO: 225 - DNA, 75 bp, Artificial
  clone of aptamer Thrombin B1
gggagaggagagaacgttctacaggtagcgtgagggggtgttaatagaggggcgctgtcgatcgatcgatcgatg

SEQ ID NO: 226 - DNA, 75 bp, Artificial
  clone of aptamer Thrombin B2
gggagaggagagaacgttctacgataggatgggtgggacaggagagggagtgcgctgtcgatcgatcgatcgatg

SEQ ID NO: 227 - DNA, 75 bp, Artificial
  clone of aptamer Thrombin B3
gggagaggagagaacgttctaccagtgagggcagtgtcagattgagaggaggcgctgtcgatcgatcgatcgatg

SEQ ID NO: 228 - DNA, 75 bp, Artificial
  clone of aptamer Thrombin B4
gggagaggagagaacgttctaccttgcctaacaggaggtggagtattggacccgctgtcgatcgatcgatcgatg

SEQ ID NO: 229 - DNA, 75 bp, Artificial; identical to SEQ ID NO: 228
  clone of aptamer Thrombin C1

SEQ ID NO: 230 - DNA, 73 bp, Artificial
  clone of aptamer Thrombin C2
gggagaggagagaacgttctacgtcgtgagtaatggctcgtagatgaggtcgctgtcgatcgatcgatcgatg

SEQ ID NO: 231 - DNA, 74 bp, Artificial
  clone of aptamer Thrombin C3
gggagaggagagaacgttctacgggattaagaggggagaggagcagttgagcgctgtcgatcgatcgatcgatg

SEQ ID NO: 232 - DNA, 75 bp, Artificial
  clone of aptamer Thrombin C4
gggagaggagagaacgttctactccggttggggtatcaggtctacggactgacgctgtcgatcgatcgatcgatg

SEQ ID NO: 233 - DNA, 75 bp, Artificial; identical to SEQ ID NO: 223
  clone of aptamer Thrombin D1

SEQ ID NO: 234 - DNA, 75 bp, Artificial; identical to SEQ ID NO: 223
  clone of aptamer Thrombin D2

SEQ ID NO: 235 - DNA, 75 bp, Artificial
  clone of aptamer Thrombin D3
gggagaggagagaacgttctacatgacaagagggggttgtgtgggatggcagcgctgtcgatcgatcgatcgatg

SEQ ID NO: 236 - DNA, 76 bp, Artificial
  clone of aptamer Thrombin D4
gggagaggagagaacgttctacacagggaggggagcggagaggagagagggtacgctgtcgatcgatcgatcgatg

SEQ ID NO: 237 - DNA, 75 bp, Artificial; identical to SEQ ID NO: 223
  clone of aptamer Thrombin E1

SEQ ID NO: 238 - DNA, 73 bp, Artificial; identical to SEQ ID NO: 230
  clone of aptamer Thrombin E2

SEQ ID NO: 239 - DNA, 75 bp, Artificial; identical to SEQ ID NO: 223
  clone of aptamer Thrombin E4

SEQ ID NO: 240 - DNA, 75 bp, Artificial; identical to SEQ ID NO: 223
  clone of aptamer Thrombin F1

SEQ ID NO: 241 - DNA, 75 bp, Artificial; identical to SEQ ID NO: 228
  clone of aptamer Thrombin F2

SEQ ID NO: 242 - DNA, 75 bp, Artificial
  clone of aptamer Thrombin F3
gggagaggagagaacgttctacggctatgcgtcgtgagtcaatggcccgcatcgctgtcgatcgatcgatcgatg

SEQ ID NO: 243 - DNA, 75 bp, Artificial
  clone of aptamer Thrombin F4
gggagaggagagaacgttctacgggtcgtgagatagtggctcccgtattcagcgctgtcgatcgatcgatcgatg

SEQ ID NO: 244 - DNA, 75 bp, Artificial; identical to SEQ ID NO: 223
  clone of aptamer Thrombin G1

SEQ ID NO: 245 - DNA, 75 bp, Artificial; identical to SEQ ID NO: 223
  clone of aptamer Thrombin G2

SEQ ID NO: 246 - DNA, 75 bp, Artificial
  clone of aptamer Thrombin G3
gggagaggagagaacgttctaccttgtctaacaggaggtggagtattggacccgctgtcgatcgatcgatcgatg

SEQ ID NO: 247 - DNA, 75 bp, Artificial
  clone of aptamer Thrombin G4
gggagaggagagaacgttctacgactttgagggtggtgagagtggaagagagcgctgtcgatcgatcgatcgatg

SEQ ID NO: 248 - DNA, 75 bp, Artificial
  clone of aptamer Thrombin H1
gggagaggagagaacgttctacggtagggtatgaccagggaggtattggaggcgctgtcgatcgatcgatcgatg

SEQ ID NO: 249 - DNA, 75 bp, Artificial; identical to SEQ ID NO: 223
  clone of aptamer Thrombin H2

SEQ ID NO: 250 - DNA, 75 bp, Artificial; identical to SEQ ID NO: 223
  clone of aptamer Thrombin H3

SEQ ID NO: 251 - DNA, 72 bp, Artificial
  clone of aptamer Thrombin H4
gggagaggagagaacgttctacgttatgcatgtggagagtgagagagggcgctgtcgatcgatcgatcgatg

SEQ ID NO: 252 - DNA, 75 bp, Artificial
  clone of aptamer VEGF A9
gggagaggagagaacgttctaccatgtctgcgggaggtgagtagtgatcctgcgctgtcgatcgatcgatcgatg

SEQ ID NO: 253 - DNA, 75 bp, Artificial
  clone of aptamer VEGF A10
gggagaggagagaacgttctacagagtgggagggatgtgtgacacaggtaggcgctgtcgatcgatcgatcgatg

SEQ ID NO: 254 - DNA, 73 bp, Artificial
  clone of aptamer VEGF A11
gggagaggagagaacgttctacgctccatgacagtgaggtgagtagtgatcgctgtcgatcgatcgatcgatg

SEQ ID NO: 255 - DNA, 74 bp, Artificial
  clone of aptamer VEGF A12
gggagaggagagaacgttctcgatgctgacagggtgtgttcagtaatggctcgctgtcgatcgatcgatcgatg

SEQ ID NO: 256 - DNA, 75 bp, Artificial
  clone of aptamer VEGF B9
gggagaggagagaacgttctaccagcaaacagggtcaggtgagtagtgatgacgctgtcgatcgatcgatcgatg

SEQ ID NO: 257 - DNA, 75 bp, Artificial
  clone of aptamer VEGF B10
gggagaggagagaacgttctacgacaagccgggggtgttcagtagtggcaaccgctgtcgatcgatcgatcgatg

SEQ ID NO: 258 - DNA, 75 bp, Artificial
  clone of aptamer VEGF B11
gggagaggagagaacgttctacatatggcgctggaggtgagtaatgatcgtgcgctgtcgatcgatcgatcgatg

SEQ ID NO: 259 - DNA, 75 bp, Artificial
  clone of aptamer VEGF B12
gggagaggagagaacgttctacggggcgatagcgttcagtagtggcgccggtcgctgtcgatcgatcgatcgatg

SEQ ID NO: 260 - DNA, 74 bp, Artificial
  clone of aptamer VEGF C9
gggagaggagagaacgttctacatagcggactgggtgcatggagcggcgcacgctgtcgatcgatcgatcgatg

SEQ ID NO: 261 - DNA, 74 bp, Artificial
  clone of aptamer VEGF C10
gggagaggagagaacgttctacgggtcaacaggggcgttcagtagtggcggcgctgtcgatcgatcgatcgatg

SEQ ID NO: 262 - DNA, 75 bp, Artificial
  clone of aptamer VEGF C11
gggagaggagagaacgttctacgcatgcgagctgaggtgagtagtgatcagtcgctgtcgatcgatcgatcgatg

SEQ ID NO: 263 - DNA, 74 bp, Artificial
  clone of aptamer VEGF C12
gggagaggagagaacgttctacatgcgacaggggagtgttcagtagtggcacgctgtcgatcgatcgatcgatg

SEQ ID NO: 264 - DNA, 75 bp, Artificial
  clone of aptamer VEGF D9
gggagaggagagaacgttctaccccatcgtatggagtgcggaacggggcatacgctgtcgatcgatcgatcgatg

SEQ ID NO: 265 - DNA, 72 bp, Artificial
  clone of aptamer VEGF D10
gggagaggagagaacgttctacagtgaggcgggagcgtttcagtaatggcgctgtcgatcgatcgatcgatg

SEQ ID NO: 266 - DNA, 74 bp, Artificial
  clone of aptamer VEGF D12
gggagaggagagaacgttctacacagcgtcgggtgttcagtaatggcgcagcgctgtcgatcgatcgatcgatg

SEQ ID NO: 267 - DNA, 75 bp, Artificial
  clone of aptamer VEGF E9
gggagaggagagaacgttctacggtgttcagtagtggcacaggaggaagggatgctgtcgatcgatcgatcgatg

SEQ ID NO: 268 - DNA, 75 bp, Artificial
  clone of aptamer VEGF E10
gggagaggagagaacgttctacagttcaggcgttaggcatgggtgtcgctttcgctgtcgatcgatcgatcgatg

SEQ ID NO: 269 - DNA, 75 bp, Artificial
  clone of aptamer VEGF E11
gggagaggagagaacgttctacatgcgacatgcgagtgttcagtagcggcagcgctgtcgatcgatcgatcgatg

SEQ ID NO: 270 - DNA, 75 bp, Artificial
  clone of aptamer VEGF E12
gggagaggagagaacgttctacctatggcgttacagcgaggtgagtagtgatcgctgtcgatcgatcgatcgatg

SEQ ID NO: 271 - DNA, 75 bp, Artificial
  clone of aptamer VEGF F9
gggagaggagagaacgttctaccagccgatccagccaggcgttcagtagtggcgctgtcgatcgatcgatcgatg

SEQ ID NO: 272 - DNA, 74 bp, Artificial
  clone of aptamer VEGF F10
gggagaggagagaacgttctacggcacaggcacggcgaggtgagtaatgatcgctgtcgatcgatcgatcgatg

SEQ ID NO: 273 - DNA, 73 bp, Artificial
  clone of aptamer VEGF G9
gggagaggagagaacgttctactgtggacagcgggagtgcggaacggggtcgctgtcgatcgatcgatcgatg

SEQ ID NO: 274 - DNA, 75 bp, Artificial
  clone of aptamer VEGF G10
gggagaggagagaacgttctactgatgctgcgagtgcatggggcaggcgcttcgctgtcgatcgatcgatcgatg

SEQ ID NO: 275 - DNA, 75 bp, Artificial
  clone of aptamer VEGF G11
gggagaggagagaacgttctacggtacaatgggaatgacagtgatgggtagccgctgtcgatcgatcgatcgatg

SEQ ID NO: 276 - DNA, 73 bp, Artificial
  clone of aptamer VEGF G12
gggagaggagagaacgttctacatggacagcgaagcatgggggaggcgcacgctgtcgatcgatcgatcgatg

SEQ ID NO: 277 - DNA, 75 bp, Artificial
  clone of aptamer VEGF H9
gggagaggagagaacgttctactgggagcgacagtgagcatggggtaggcgccgctgtcgatcgatcgatcgatg

SEQ ID NO: 278 - DNA, 74 bp, Artificial
  clone of aptamer VEGF H11
gggagaggagagaacgttctaccggcgagcaggtgttcagtagtggctttgcgctgtcgatcgatcgatcgatg

SEQ ID NO: 279 - DNA, 75 bp, Artificial
  clone of aptamer VEGF H12
gggagaggagagaacgttctacgatcagtgagggagtgcagtagtggctcgtcgctgtcgatcgatcgatcgatg

SEQ ID NO: 280 - DNA, 81 bp, Artificial
  clone of aptamer ARX34P2.G01
gggagaggagagaacgttctacaaatgagagcaggccgaaaaggagtcgctcgctgtcgatcgatcgatcgatgaagggcg

SEQ ID NO: 281 - DNA, 82 bp, Artificial
  clone of aptamer ARX34P2.A06
gggagaggagagaacgttctacaaaggatcaatctttcggcgtatgtgtgagcgctgtcgatcgatcgatcgatgaagggcg

SEQ ID NO: 282 - DNA, 82 bp, Artificial
  clone of aptamer ARX34P2.E02
gggagaggagagaacgttctacggtaaagcaggctgactgaaaggttgaagtcgctgtcgatcgatcgatcgatgaagggcg

SEQ ID NO: 283 - DNA, 81 bp, Artificial
  clone of aptamer ARX34P2.H05
gggagaggagagaacgttctacaggttaaaagcaggctcaggaatggaagtcgctgtcgatcgatcgatcgatgaagggcg

SEQ ID NO: 284 - DNA, 82 bp, Artificial
  clone of aptamer ARX34P2.G04
gggagaggagagaacgttctacaacaaagcaggctcatagtaatatggaagtcgctgtcgatcgatcgatcgatgaagggcg

SEQ ID NO: 285 - DNA, 81 bp, Artificial
  clone of aptamer ARX34P2.G03
gggagaggagagaacgttctacaaaagagagcaggccgaaaaggagtcgctcgctgtcgatcgatcgatcgatgaagggcg

SEQ ID NO: 286 - DNA, 82 bp, Artificial
  clone of aptamer ARX34P2.H06
gggagaggagagaacgttctacaaaaggcaggctcaggggatcactggaagtcgctgtcgatcgatcgatcgatgaagggcg

SEQ ID NO: 287 - DNA, 82 bp, Artificial
  clone of aptamer ARX34P2.B01
gggagaggagagaacgttctacaaaaagcaggccgtatggatataagggagtcgctgtcgatcgatcgatcgatgaagggcg

SEQ ID NO: 288 - DNA, 82 bp, Artificial
  clone of aptamer ARX34P2.B03
gggagaggagagaacgttctacaaaagtgcaggctgcagacatatgcgaagtcgctgtcgatcgatcgatcgatgaagggcg

SEQ ID NO: 289 - DNA, 81 bp, Artificial
  clone of aptamer ARX34P2.D05
gggagaggagagaacgttctacaaaggagagcaggccgaaaaggagtcgctcgctgtcgatcgatcgatcgatgaagggcg

SEQ ID NO: 290 - DNA, 83 bp, Artificial
  clone of aptamer ARX34P2.C05
gggagaggagagaacgttctacaagatataattaaggataagtgcaaaggagacgctgtcgatcgatcgatcgatgaagggcg

SEQ ID NO: 291 - DNA, 84 bp, Artificial
  clone of aptamer ARX34P2.C04
gggagaggagagaacgttctacagacaacagcnagagggaatcncanacaaagacgctgtcgatcgatcgatcgatgaagggcg

SEQ ID NO: 292 - DNA, 82 bp, Artificial
  clone of aptamer ARX34P2.E06
gggagaggagagaacgttctacagattctaagcgcaggaataagtcaccagacgctgtcgatcgatcgatcgatgaagggcg

SEQ ID NO: 293 - DNA, 82 bp, Artificial
  clone of aptamer ARX34P2.A01
gggagaggagagaacgttctacgaaaatgagcatggaagtgggagtacgtgccgctgtcgatcgatcgatcgatgaagggcg

SEQ ID NO: 294 - DNA, 82 bp, Artificial
  clone of aptamer ARX34P2.C06
gggagaggagagaacgttctacgaaaagaggcgccggaagtgagagtaagtgcgctgtcgatcgatcgatcgatgaagggcg

SEQ ID NO: 295 - DNA, 82 bp, Artificial
  clone of aptamer ARX34P2.B04
gggagaggagagaacgttctacgaagtgagtttccgaagtgagagtacgaaacgctgtcgatcgatcgatcgatgaagggcg

SEQ ID NO: 296 - DNA, 81 bp, Artificial
  clone of aptamer ARX34P2.E04
gggagaggagagaacgttctacgaatgagagcaggccgaaaaggagtcgctcgctgtcgatcgatcgatcgatgaagggcg

SEQ ID NO: 297 - DNA, 82 bp, Artificial
  clone of aptamer ARX34P2.H04
gggagaggagagaacgttctacgagaggcaagagagagtcgcataaaaaagacgctgtcgatcgatcgatcgatgaagggcg

SEQ ID NO: 298 - DNA, 82 bp, Artificial
  clone of aptamer ARX34P2.B06
gggagaggagagaacgttctacgcaggctgtcgtagacaaacgatgaagtcgcgctgtcgatcgatcgatcgatgaagggcg

SEQ ID NO: 299 - DNA, 83 bp, Artificial
  clone of aptamer ARX34P2.F05
gggagaggagagaacgttctacggaaaaagatatgaaagaaaggattaagagacgctgtcgatcgatcgatcgatgaagggcg

SEQ ID NO: 300 - DNA, 82 bp, Artificial
  clone of aptamer ARX34P2.H02
gggagaggagagaacgttctacggaaggnaacaanagcactgtttgtgcaggcgctgtcgatcnatcnatcnatgaagggcg

SEQ ID NO: 301 - DNA, 82 bp, Artificial
  clone of aptamer ARX34P2.C03
gggagaggagagaacgttctacggagcatanggcntgaaactgaganagtaacgctgtcgatcgatcgatcgatgaagggcg

SEQ ID NO: 302 - DNA, 83 bp, Artificial
  clone of aptamer ARX34P2.D01
gggagaggagagaacgttctacgaaaaaggatatgagagaaaggattaagagacgctgtcgatcgatcgatcgatgaagggcg

SEQ ID NO: 303 - DNA, 82 bp, Artificial
  clone of aptamer ARX34P2.A03
gggagaggagagaacgttctacatacataggcgccgcgaatgggaaagaaagcgctgtcgatcgatcgatcgatgaagggcg

SEQ ID NO: 304 - DNA, 82 bp, Artificial
  clone of aptamer ARX34P2.B02
gggagaggagagaacgttctactcatgaagccatggttgtaattctgtttggcgctgtcgatcgatcgatcgatgaagggcg

SEQ ID NO: 305 - DNA, 80 bp, Artificial
  clone of aptamer ARX34P2.C01
gggagaggagagaacgttctactaatgcaggctcagttactactggaagtcgctgtcgatcgatcgatcgatgaagggcg

SEQ ID NO: 306 - DNA, 81 bp, Artificial
  clone of aptamer ARX34P2.D06
gggagaggagagaacgttctactttcataggcgggattatggaggagtattcgctgtcgatcgatcgatcgatgaagggcg

SEQ ID NO: 307 - DNA, 82 bp, Artificial
  clone of aptamer ARX34P2.G05
aggagaggagagaacgttctactagaagcaggctcgaatacaattcggaagtcgctgtcgatcgatcgatcgatgaagggcg

SEQ ID NO: 308 - DNA, 82 bp, Artificial
  clone of aptamer ARX34P2.F06
gggagaggagagaacgttctacttagcgatgtcggaagagagagtacgaggacgctgtcgatcgatcgatcgatgaagggcg

SEQ ID NO: 309 - DNA, 82 bp, Artificial
  clone of aptamer ARX34P2.F02
gggagaggagagaacgttctacttgcgaagaccgtggaagaggagtactggtcgctgtcgatcgatcgatcgatgaagggcg

SEQ ID NO: 310 - DNA, 82 bp, Artificial
  clone of aptamer ARX34P2.B05
gggagaggagagaacgttctacttttggtgaaggtgtaagagtggcactacacgctgtcgatcgatcgatcgatgaagggcg

SEQ ID NO: 311 - DNA, 82 bp, Artificial
  clone of aptamer ARX34P2.A05
gggagaggagagaacgttctaccatcagttgtggcgattatgtgggagtatgcgctgtcgatcgatcgatcgatgaagggcg

SEQ ID NO: 312 - DNA, 83 bp, Artificial
  clone of aptamer ARX34P2.E03
gggagaggagagaacgttctacanaanaacatgcgattaaagatcatgaacagcgctgtcgatcgatcgatcgatgaagggcg

SEQ ID NO: 313 - DNA, 82 bp, Artificial
  clone of aptamer ARX34P2.F04
gggagaggagagaacgttctacataagcaggctccgatagtattcgggaagtcgctgtcgatcgatcgatcgatgaagggcg

SEQ ID NO: 314 - DNA, 82 bp, Artificial
  clone of aptamer ARX34P2.E10
gggagaggagagaacgttctactttcggaatgcgatgggggtgattcgtggtcgctgtcgatcgatcgatcgatgaagggcg

SEQ ID NO: 315 - DNA, 80 bp, Artificial
  clone of aptamer ARX34P2.H09
gggagaggagagaacgttctacctgttgaggctaagtggatgattgagggcgctgtcgatcgatcgatcgatgaagggcg

SEQ ID NO: 316 - DNA, 81 bp, Artificial
  clone of aptamer ARX34P2.A07
gggagaggagagaacgttctacctgggtcggtgcgattggagatgtcgttgcgctgtcgatcgatcgatcgatgaagggcg

SEQ ID NO: 317 - DNA, 82 bp, Artificial
  clone of aptamer ARX34P2.A12
gggagaggagagaacgttctacctgatgtcaggttgtttggagattatctgacnctgtcnatcgatcgatcgatgaagggcg

SEQ ID NO: 318 - DNA, 82 bp, Artificial
  clone of aptamer ARX34P2.A08
gggagaggagagaacgttctacctcgcgcgacgagcgaatttccggatgcggcgctgtcgatcgatcgatcgatgaagggcg

SEQ ID NO: 319 - DNA, 82 bp, Artificial
  clone of aptamer ARX34P2.D12
gggagaggagagaacgttctaccatgaatgattgcgatcgttgttcgtgtggcgctgtcgatcgatcgatcgatgaagggcg

SEQ ID NO: 320 - DNA, 83 bp, Artificial
  clone of aptamer ARX34P2.E11
gggagaggagagaacgttctactccgaccacgcctgggtgattcctacnacgacgctgtcgatcgatcgatcgatgaagggcg

SEQ ID NO: 321 - DNA, 82 bp, Artificial
  clone of aptamer ARX34P2.E12
gggagaggagagaacgttctactacttttggggattcactccgcgctgatgtcgctgtcgatcgatcgatcgatgaagggcg

SEQ ID NO: 322 - DNA, 82 bp, Artificial
  clone of aptamer ARX34P2.D08
gggagaggaganaacgttctantagtgcttgcgagatagtgtaggattatactgctgtcgatcgatcgatcgatgaagggcg

SEQ ID NO: 323 - DNA, 82 bp, Artificial
  clone of aptamer ARX34P2.F07
gggagaggagagaacgttctactagtgtccttctccacgtggttgtaattgtcgctgtcgatcgatcgatcgatgaagggcg

SEQ ID NO: 324 - DNA, 82 bp, Artificial
  clone of aptamer ARX34P2.B11
gggagaggagagaacgttctactattgtggcgcttgttggactaactgactacgctgtcgatcgatcgatcgatgaagggcg

SEQ ID NO: 325 - DNA, 82 bp, Artificial
  clone of aptamer ARX34P2.F12
gggagaggagagaacgtcctacttcgattgtgatcttgtggcggcctgtgagcgctgtcgatcgatcgatcgatgaagggcg

SEQ ID NO: 326 - DNA, 82 bp, Artificial
  clone of aptamer ARX34P2.A09
gggagaggagagaacgttctacttggcgatgtcggaagagagagtacgagggcgctgtcgatcgatcgatcgatgaagggcg

SEQ ID NO: 327 - DNA, 82 bp, Artificial
  clone of aptamer ARX34P2.D07
gggagaggagagaacgttctacttgaanctgcgtgaattganagtaacgaagcgctgtcaatcgatcnatcaatnaagggcg

SEQ ID NO: 328 - DNA, 82 bp, Artificial
  clone of aptamer ARX34P2.H10
gggagaggagagaacgttctactcgagaggacatgtggatccggttcgcgtgcgctgtcgatcgatcgatcgatgaagggcg

SEQ ID NO: 329 - DNA, 82 bp, Artificial
  clone of aptamer ARX34P2.H07
gggagaggagagaacgttctactgtgatgcggtttgcgtcgaccggattcgtcgctgtcgatcgatcgatcgatgaagggcg

SEQ ID NO: 330 - DNA, 81 bp, Artificial
  clone of aptamer ARX34P2.F11
gggagaggagagaacgttctactgtgtgattgggcgcatgtcgaggcgacacgctgtcgatcgatcgatcgatgaagggcg

SEQ ID NO: 331 - DNA, 81 bp, Artificial
  clone of aptamer ARX34P2.G07
gggagaggagagaacgttctactgattaagatgcgctggtagagcggtgggcgctgtcgatcgatcgatcgatgaagggcg

SEQ ID NO: 332 - DNA, 82 bp, Artificial
  clone of aptamer ARX34P2.A10
gggagaggagagaacgttctactggttaatttgcatgcgcgantaacntgntcgctgtcgatcgatcgatcgatgaagggcg

SEQ ID NO: 333 - DNA, 81 bp, Artificial
  clone of aptamer ARX34P2.G10
gggagaggagagaacgttctactgggaagcggtaacttggattgaccgatccgctgtcgatcgatcgatcgatgaagggcg

SEQ ID NO: 334 - DNA, 82 bp, Artificial
  clone of aptamer ARX34P2.H11
gggagaggagagaacgttctactgttacggagatgatgggtttggctgttggcgctgtcgatcgatcgatcgatgaagggcg

SEQ ID NO: 335 - DNA, 81 bp, Artificial
  clone of aptamer ARX34P2.C07
gggagaggagagaacgttctacttgtggactgagatacgattcggagctggcgctgtcgatcgatcgatcgatgaagggcg

SEQ ID NO: 336 - DNA, 82 bp, Artificial
  clone of aptamer ARX34P2.E08
gggagaggagagaacgttctacttgtgagtttccttgggccttgagcgtgggcgctgtcgatcgatcgatcgatgaagggcg

SEQ ID NO: 337 - DNA, 82 bp, Artificial
  clone of aptamer ARX34P2.A11
gggagaggagagaacgttctacaggtgatgtgagccgattgtgaagttttgtcgctgtcgatcgatcgatcgatgaagggcg

SEQ ID NO: 338 - DNA, 82 bp, Artificial
  clone of aptamer ARX34P2.B08
gggagaggagagaacgttctacagcggatgtttgggggtgtgtgttggttgtcgctgtcgatcgatcgatcgatgaagggcg

SEQ ID NO: 339 - DNA, 82 bp, Artificial
  clone of aptamer ARX34P2.B09
gggagaggagagaacgttctacatgcggtggtggtcttcgatgggtggaagtcgctgtcgatcgatcgatcgatgaagggcg

SEQ ID NO: 340 - DNA, 82 bp, Artificial
  clone of aptamer ARX34P2.B12
gggagaggagagaacgttctacattggaggggcgcatgtggtctgtttgatgcgctgtcgatcgatcgatcgatgaagggcg

SEQ ID NO: 341 - DNA, 82 bp, Artificial
  clone of aptamer ARX34P2.F10
gggagaggagagaacgttctacgtgtttcgcggatttgaagaggagtaaaatcgctgtcgatcgatcgatcgatgaagggcg

SEQ ID NO: 342 - DNA, 82 bp, Artificial
  clone of aptamer ARX34P2.B10
gggagaggagagaacgttctacgtgtgcgtgttcgggaagggagagtgccgaggctgtcgatcgatcgatcgatgaagggcg

SEQ ID NO: 343 - DNA, 82 bp, Artificial
  clone of aptamer ARX34P2.G08
gggagaggagagaacgttctacgtgtgtggtgtgcgatgcttggctgtttgtcgctgtcgatcgatcgatcgatgaagggcg

SEQ ID NO: 344 - DNA, 82 bp, Artificial
  clone of aptamer ARX34P2.C08
gggagaggagagaacgttctacggtttgtgtggcttggatctgaagactaagcgctgtcgatcgatcgatcgatgaagggcg

SEQ ID NO: 345 - DNA, 82 bp, Artificial
  clone of aptamer ARX34P2.F09
gggagaggagagaacgttctacggttctgggcttgtgtgtgaggattgacggcgctgtcgatcgatcgatcgatgaagggcg

SEQ ID NO: 346 - DNA, 74 bp, Artificial
  clone of aptamer ARX34P2.C10
gggagaggagagaacgttctacgatgatgaaggcgaaaagacgaggctgtcgatcgatcgatcgatgaagggcg

SEQ ID NO: 347 - DNA, 82 bp, Artificial
  clone of aptamer ARX34P2.C11
gggagaggagagaacgttctacgagtgctgatgcgtgtcctgggatggaattcgctgtcgatcgatcgatcgatgaagggcg

SEQ ID NO: 348 - DNA, 82 bp, Artificial
  clone of aptamer ARX34P2.D09
gggagaggagagaacgttctacgcgtttatagcgatcgatgatgatataggccgctgtcgatcgatcgatcgatgaagggcg

SEQ ID NO: 349 - DNA, 82 bp, Artificial
  clone of aptamer ARX34P2.D10
gggagaggagagaacgttctacgcgttcaaatgggatagaattggctgcgggcgctgtcgatcgatcgatcgatgaagggcg

SEQ ID NO: 350 - DNA, 79 bp, Artificial
  clone of aptamer ARX34P2.D11
gggagaggagagaacgttctacgaaattgtgcgtcagtgtgaggcggtttgctgtcgatcgatcgatcgatgaagggcg

SEQ ID NO: 351 - DNA, 82 bp, Artificial
  clone of aptamer ARX34P2.E07
gggagaggagagaacgttctacggtcgaaatgagggtctggagttccgacgacgctgtcgatcgatcgatcgatgaagggcg

SEQ ID NO: 352 - DNA, 82 bp, Artificial
  clone of aptamer ARX34P2.E09
gggagaggagagaacgttctacgaatttggtaatctgggtgacttaggatgtcgctgtcgatcgatcgatcgatgaagggcg

SEQ ID NO: 353 - DNA, 81 bp, Artificial
  clone of aptamer ARX34P2.G12
gggagaggagagaacgttctacgattttttgtgccgaagtaagagtacgcgcgctgtcgatcgatcgatcgatgaagggcg

SEQ ID NO: 354 - DNA, 82 bp, Artificial
  clone of aptamer ARX34P2.H08
aggagaggagagaacgttctacggagtgtgcgcggatgaaaacagaagttgtcgctgtcnatcgatcnatcaatgaagggcg

SEQ ID NO: 355 - DNA, 82 bp, Artificial
  clone of aptamer AMX(123).A1
gggagaggagagaacgttctacgatctgggcgagccagtctgactgaggaagcgctgtcgatcgatcgatcgatgaagggcg

SEQ ID NO: 356 - DNA, 82 bp, Artificial
  clone of aptamer ARX34P1.B07
gggagaggagagaacgttctacgaagaagatatgagagaaaggattaagagacgctgtcgatcgatcgatcgatgaagggcg

SEQ ID NO: 357 - DNA, 82 bp, Artificial
  clone of aptamer ARX34P1.A07
gggagaggagagaacgttctacgaaaaagatatgagagaaaggattaagagacgctgtcgatcgatcgatcgatgaagggcg

SEQ ID NO: 358 - DNA, 82 bp, Artificial
  clone of aptamer ARX34P1.A01
gggagaggagagaacgttctacgaaaaagatatgagagaaaggattaagaggcgctgtcgatcgatcgatcgatgaagggcg

SEQ ID NO: 359 - DNA, 82 bp, Artificial
  clone of aptamer ARX34P1.G05
gggagaggagagaacgttctacgaaaaagacatgagagaaaggattaagagacgctgtcgatcgatcgatcgatgaagggcg

SEQ ID NO: 360 - DNA, 83 bp, Artificial
  clone of aptamer ARX34P1.F09
gggagaggagagaacgttctacnaaaaagtatatgagagaaaggattaanagacgctgtcgatcgatcgatcgatgaagggcg

SEQ ID NO: 361 - DNA, 83 bp, Artificial
  clone of aptamer ARX34P1.B02
gggagaggagagaacgttctacgaaaaagatatgagagaaaaggattgagagatgctgtcgatcgatcgatcgatgaagggcg

SEQ ID NO: 362 - DNA, 83 bp, Artificial
  clone of aptamer ARX34P1.G02
gggagaggagagcacgttctacgaaaaagatatggagagaaaggattaagagacgctgtcgatcgatcgatcgatgaagggcg

SEQ ID NO: 363 - DNA, 84 bp, Artificial
  clone of aptamer ARX34P1.A04
gggagaggagagaacgttctacgaaaaagatatgagagaaaggattaaaagagacgctgtcgatcgatcgatcgatgaagggcg

SEQ ID NO: 364 - DNA, 85 bp, Artificial
  clone of aptamer ARX34P1.G06
gggagaggagagaacgttctacgaanaagatacatagtagaaaggattaataagacgctgtcgatcgatcgatcgatgaagggcg

SEQ ID NO: 365 - DNA, 82 bp, Artificial
  clone of aptamer ARX34P1.E05
gggagaggagagaacgttctacaggcgtgttggtagggtacgacgaggcatgcgctgtcgatcgatcgatcgatgaagggcg

SEQ ID NO: 366 - DNA, 82 bp, Artificial
  clone of aptamer ARX34P1.B11
gggagaggagagaacgttctacgcaaaaatgtgatgcgaggtaatggaacgccgctgtcgatcgatcgatcgatgaagggcg

SEQ ID NO: 367 - DNA, 82 bp, Artificial
  clone of aptamer ARX34P1.B01
gggagaggagagaacgttctacggacctcagcgataggggttgaaaccgacacgctgtcgatcgatcgatcgatgaagggcg

SEQ ID NO: 368 - DNA, 82 bp, Artificial
  clone of aptamer ARX34P1.H06
gggagaggagagaacgttctacatggtcggatgctggggagtaggcaaggttcgctgtcgatcgatcgatcgatgaagggcg

SEQ ID NO: 369 - DNA, 82 bp, Artificial
  clone of aptamer ARX34P1.C12
gggagaggagagaacgttctacgtatcggcgagcgaagcatccgggagcgttcgctgtcgatcgatcgatcgatgaagggcg

SEQ ID NO: 370 - DNA, 82 bp, Artificial
  clone of aptamer ARX34P1.C09
gggagaggagagaacgttctacgtattggcgcgcgaagcatccgggagcgttcgctgtcgatcgatcgatcgatgaagggcg

SEQ ID NO: 371 - DNA, 82 bp, Artificial
  clone of aptamer ARX34P1.A11
gggagaggagagaacgttctacttatacctgacggccggaggcgcataggtgcgctgtcgatcgatcgatcgatgaagggcg

SEQ ID NO: 372 - DNA, 82 bp, Artificial; identical to SEQ ID NO: 368
  clone of aptamer ARX34P1.H09

SEQ ID NO: 373 - DNA, 82 bp, Artificial
  clone of aptamer ARX34P1.B05
gggagaggagagaacgttctacacgagagtactgaggcgcttggtacagagtcgctgtcgatcgatcgatcgatgaagggcg

SEQ ID NO: 374 - DNA, 82 bp, Artificial
  clone of aptamer ARX34P1.B10
gggagaggagagaacgttctacagaaggtagaaaaaggatagctgtgagaagcgctgtcgatcgatcgatcgatgaagggcg

SEQ ID NO: 375 - DNA, 82 bp, Artificial
  clone of aptamer ARX34P1.C01
gggagaggagagaacgttctactgagggataatacgggtgggattgtcttcccgctgtcgatcgatcgatcgatgaagggcg

SEQ ID NO: 376 - DNA, 84 bp, Artificial
  clone of aptamer ARX34P1.D04
gggagaggagagaacgttctacattgagcgttgaagttggggaagctccgaggccgctgtcgatcgatcgatcgatgaagggcg

SEQ ID NO: 377 - DNA, 82 bp, Artificial
  clone of aptamer ARX34P1.E02
gggagaggagagaacgttctacgcggagatatacagcgaggtaatggaacgccgctgtcgatcgatcgatcgatgaagggcg

SEQ ID NO: 378 - DNA, 82 bp, Artificial
  clone of aptamer ARX34P1.F01
gggagaggagagaacgttctacgaagacagcccaatagcggcacggaacttgcgctgtcgatcgatcgatcgatgaagggcg

SEQ ID NO: 379 - DNA, 84 bp, Artificial
  clone of aptamer ARX34P1.G03
gggagaggagagaacgttctaccggttgagggctcgcgtggaagggccaacacgcgctgtcgatcgatcgatcgatgaagggcg

SEQ ID NO: 380 - DNA, 82 bp, Artificial
  clone of aptamer ARX34P1.H01
gggagaggagagaacgttctacatatcaatagactcttgacgtttgggtttgcgctgtcgatcgatcgatcgatgaagggcg

SEQ ID NO: 381 - DNA, 79 bp, Artificial
  clone of aptamer ARX34P1.H02
gggagaggagagaacgttctacagtgaaggaaaagtaagtgaaggtgtgcgctgtcgatcgatcgatcgatgaagggcg

SEQ ID NO: 382 - DNA, 82 bp, Artificial
  clone of aptamer ARX34P1.H03
gggagaggagagaacgttctacggatgaaatgagtgtctgcgataggttaagcgctgtcgatcgatcgatcgatgaagggcg

SEQ ID NO: 383 - DNA, 83 bp, Artificial
  clone of aptamer ARX34P1.H10
gggagaggagagaacgttctacggaaggaaatgtgtgtctgcgataggttaagcgctgtcgatcgatcgatcgatgaagggcg

SEQ ID NO: 384 - DNA, 82 bp, Artificial
  clone of aptamer PDGF-BB  ARX36.SCK.E05
gggagaggagagaacgttctacatccttgcgtatgatcggcatcgtaagacacgctgtcgatcgatcgatcgatgaagggcg

SEQ ID NO: 385 - DNA, 82 bp, Artificial; identical to SEQ ID NO: 384
  clone of aptamer PDGF-BB  ARX36.SCK.F05

SEQ ID NO: 386 - DNA, 77 bp, Artificial
  clone of aptamer PDGF-BB  ARX36.SCK.E01
gggagaggagagaacgttctacgatcgaagtcgtgacagaaaccactcgctgtcgatcgatcgatcgatgaagggcg

SEQ ID NO: 387 - DNA, 77 bp, Artificial; identical to SEQ ID NO: 386
  clone of aptamer PDGF-BB  ARX36.SCK.F01

SEQ ID NO: 388 - DNA, 82 bp, Artificial
  clone of aptamer PDGF-BB  ARX36.SCK.G01
gggagaggagagaacgttctacggaaaaggttggcgaaacgaagaagaatttcgctgtcgatcgatcgatcgatgaagggcg

SEQ ID NO: 389 - DNA, 82 bp, Artificial
  clone of aptamer PDGF-BB  ARX36.SCK.G02
gggagaggagagaacgttctacggaaaaggttggcgaaacgaagaanaatttcgctgtcgatcgatcgatcgatgaagggcg

SEQ ID NO: 390 - DNA, 83 bp, Artificial
  clone of aptamer PDGF-BB  ARX36.SCK.F04
gggagaggagagaacgttctactgggagttgcggtgttttgcggtggatttgacgctgtcgatcgatcgatcgatgaagggcg

SEQ ID NO: 391 - DNA, 83 bp, Artificial; identical to SEQ ID NO: 390
  clone of aptamer PDGF-BB  ARX36.SCK.E04

SEQ ID NO: 392 - DNA, 82 bp, Artificial
  clone of aptamer PDGF-BB  ARX36.SCK.F02
gggagaggagagaacgctctacaagattgtagatcaacagcgaaggcgtgggcgctgtcgatcgatcgatcgatgaagggcg

SEQ ID NO: 393 - DNA, 82 bp, Artificial; identical to SEQ ID NO: 392
  clone of aptamer PDGF-BB  ARX36.SCK.E02

SEQ ID NO: 394 - DNA, 82 bp, Artificial
  clone of aptamer PDGF-BB  ARX36.SCK.A02
gggagaggagagaacgttctacaaanaagatnnccancnngaganaaaggagcgctgtcgatcgatcgatcgatgaagggcg

SEQ ID NO: 395 - DNA, 82 bp, Artificial
  clone of aptamer PDGF-BB  ARX36.SCK.A03
gggagaggagagaacgttctacaaacatcgaagatcgaactgaaaaggagggcgctgtcgatcgatcgatcgatgaagggcg

SEQ ID NO: 396 - DNA, 82 bp, Artificial
  clone of aptamer PDGF-BB  ARX36.SCK.A06
gggagaggagagaacgttctacatgtgcatgcaaggtggggctgacacgagccgctgtcgatcgatcgatcgatgaagggcg

SEQ ID NO: 397 - DNA, 80 bp, Artificial
  clone of aptamer PDGF-BB  ARX36.SCK.B01
gggagaggagagaacgttctacaaggagtagatcgacagaatagaaaaatcgctgtcgatcgatcgatcgatgaagggcg

SEQ ID NO: 398 - DNA, 83 bp, Artificial
  clone of aptamer PDGF-BB  ARX36.SCK.B02
gggagaggagagaacgttctacaaaaggtaaggtcaaaaaagcgcaacgttgacgctgtcgatcgatcgatcgatgaagggcg

SEQ ID NO: 399 - DNA, 82 bp, Artificial
  clone of aptamer PDGF-BB  ARX36.SCK.D04
gggagaggagagaacgttctacaaaaggaggcgaaataagtgagacaatgtgcgctgtcgatcgatcgatcgatgaagggcg

SEQ ID NO: 400 - DNA, 81 bp, Artificial
  clone of aptamer PDGF-BB  ARX36.SCK.B04
gggagaggagagaacgttctacaaaaatccacaaacatagctgtaattgctcgctgtcgatcgatcgatcgatgaagggcg

SEQ ID NO: 401 - DNA, 81 bp, Artificial
  clone of aptamer PDGF-BB  ARX36.SCK.B05
gggagaggagagacgttctacaagaacatataacattttggttgagagcaacgctgtcgatcgatcgatcgatgaagggcg

SEQ ID NO: 402 - DNA, 83 bp, Artificial
  clone of aptamer PDGF-BB  ARX36.SCK.D03
gggagaggagagaacgttctacaagagtcnacgatttcnatcacaaatgtggctgctgtcnatcgatcgatcnatgaagggcg

SEQ ID NO: 403 - DNA, 83 bp, Artificial
  clone of aptamer PDGF-BB  ARX36.SCK.C01
gggagaggagagaacgttctacaagcaagcaaaaaaagtatcgacagaagtggcgctgtcgatcgatcgatcgatgaagggcg

SEQ ID NO: 404 - DNA, 82 bp, Artificial
  clone of aptamer PDGF-BB  ARX36.SCK.D06
gggagaggagagaacgttctacaagtaatatcagagcaatcggaataagagtcgctgtcgatcgatcgatcgatgaagggcg

SEQ ID NO: 405 - DNA, 82 bp, Artificial
  clone of aptamer PDGF-BB  ARX36.SCK.D02
gggagaggagagaacgttctacagacttcgatgcgatggatttggaaatgtgcgctgtcgatcgatcgatcgatgaagggcg

SEQ ID NO: 406 - DNA, 82 bp, Artificial
  clone of aptamer PDGF-BB  ARX36.SCK.C03
gggagaggagagaacgttctacagaaagaattacaggaacaaatacacgtgcggctgtcgatcgatcgatcgatgaagggcg

SEQ ID NO: 407 - DNA, 82 bp, Artificial
  clone of aptamer PDGF-BB  ARX36.SCK.F06
gggagaggagagaacgttctacagaaatcaatcgaggtgatcgttatataggcgctgtcgatcgatcgatcgatgaagggcg

SEQ ID NO: 408 - DNA, 82 bp, Artificial
  clone of aptamer PDGF-BB  ARX36.SCK.C04
gggagaggagagaacgttctacagatttggatcgacaatctcgtagaagagacgctgtcgatcgatcgatcgatgaagggcg

SEQ ID NO: 409 - DNA, 82 bp, Artificial
  clone of aptamer PDGF-BB  ARX36.SCK.C06
gggagaggagagaacgttctacaatgcaagtttaagtgtggtgtcaaacgcacgctgtcgatcgatcgatcgatgaagggcg

SEQ ID NO: 410 - DNA, 81 bp, Artificial
  clone of aptamer PDGF-BB  ARX36.SCK.G03
gggagaggagagaacgttctacaaataaagacacgaagatcgacggagactcgctgtcgatcgatcgatcgatgaagggcg

SEQ ID NO: 411 - DNA, 82 bp, Artificial
  clone of aptamer PDGF-BB  ARX36.SCK.F03
gggagaggagagaacgttctacgaagatgtgtttaagaatcgaggttttcgacgctgtcgatcgatcgatcgatgaagggcg

SEQ ID NO: 412 - DNA, 81 bp, Artificial
  clone of aptamer PDGF-BB  ARX36.SCK.C02
gggagaggagagaacgttctacgagttggcacgcatgtataggtattttggcgctgtcgatcgatcgatcgatgaagggcg

SEQ ID NO: 413 - DNA, 84 bp, Artificial
  clone of aptamer PDGF-BB  ARX36.SCK.B03
gggagaggagagaacgttctacgaaaaaaagagatgagagaaaggattaagagacgctgtcgatcgatcgatcgatgaagggcg

SEQ ID NO: 414 - DNA, 82 bp, Artificial
  clone of aptamer PDGF-BB  ARX36.SCK.B06
gggagaggagagaacgttctacgaaaaggaaaaaaaacgatcggcagagtcccgctgtcgatcgatcgatcgatgaagggcg

SEQ ID NO: 415 - DNA, 82 bp, Artificial
  clone of aptamer PDGF-BB  ARX36.SCK.C05
gggagaggagagaacgttctacgattaaggaaacatttacgcgaatacatgacgctgtcgatcgatcgatcgatgaagggcg

SEQ ID NO: 416 - DNA, 81 bp, Artificial
  clone of aptamer PDGF-BB  ARX36.SCK.D01
gggagaggagagaacgttctacgacgtttgctctgaaaataggacagaaggcgctgtcgatcgatcgatcgatgaagggcg

SEQ ID NO: 417 - DNA, 82 bp, Artificial; identical to SEQ ID NO: 411
  clone of aptamer PDGF-BB  ARX36.SCK.E03

SEQ ID NO: 418 - DNA, 82 bp, Artificial
  clone of aptamer PDGF-BB  ARX36.SCK.A04
gggagaggagagaacgttctaccgagatcgaaaggtaagagaaaattcatggcgctgtcgatcgatcgatcgatgaagggcg

SEQ ID NO: 419 - DNA, 82 bp, Artificial
  clone of aptamer PDGF-BB  ARX36.SCK.A05
gggagaggagagaacgttctactaagattcgtcgttcagacagagaaagcgacgctgtcgatcgatcgatcgatgaagggcg

SEQ ID NO: 420 - DNA, 84 bp, Artificial
  clone of aptamer PDGF-BB  ARX36.SCK.E08.M13F
gggagaggagagaacgttctaccttggcgacgatctgtgacctgaatttttgtccgctgtcgatcgatcgatcgatgaagggcg

SEQ ID NO: 421 - DNA, 84 bp, Artificial; identical to SEQ ID NO: 420
  clone of aptamer PDGF-BB  ARX36.SCK.F08.M13F

SEQ ID NO: 422 - DNA, 83 bp, Artificial
  clone of aptamer ARX36.SCK.E09.M13F
gggagaggagagaacgttctaccttggtctcagcagcttttaacaaagtatcccgctgtcgatcgatcgatcgatgaagggcg

SEQ ID NO: 423 - DNA, 83 bp, Artificial; identical to SEQ ID NO: 422
  clone of aptamer PDGF-BB  ARX36.SCK.F09.M13F

SEQ ID NO: 424 - DNA, 81 bp, Artificial
  clone of aptamer PDGF-BB  ARX36.SCK.F07.M13F
gggagaggagagaacgttctaccgctattttgttcattgaaggacttgtcacgctgtcgatcgatcgatcgatgaagggcg

SEQ ID NO: 425 - DNA, 81 bp, Artificial; identical to SEQ ID NO: 424
  clone of aptamer PDGF-BB  ARX36.SCK.E07.M13F

SEQ ID NO: 426 - DNA, 82 bp, Artificial
  clone of aptamer PDGF-BB  ARX36.SCK.E11.M13F
gggagaggagagaacgttctaccctattgaggttgattggaagtgcctatgtcgctgtcgatcgatcgatcgatgaagggcg

SEQ ID NO: 427 - DNA, 82 bp, Artificial; identical to SEQ ID NO: 426
  clone of aptamer PDGF-BB  ARX36.SCK.F11.M13F

SEQ ID NO: 428 - DNA, 81 bp, Artificial
  clone of aptamer PDGF-BB  ARX36.SCK.F10.M13F
gggagaggagagaacgttctactgaagatgttatgatgattgacgaggaggcgctgtcgatcgatcgatcgatgaagggcg

SEQ ID NO: 429 - DNA, 81 bp, Artificial; identical to SEQ ID NO: 428
  clone of aptamer PDGF-BB  ARX36.SCK.E10.M13F

SEQ ID NO: 430 - DNA, 82 bp, Artificial
  clone of aptamer PDGF-BB  ARX36.SCK.E12.M13F
gggagaggagagaacgttctactgtctgagtgtcgccgccttgtgtgatgttcgctgtcgatcgatcgatcgatgaagggcg

SEQ ID NO: 431 - DNA, 82 bp, Artificial; identical to SEQ ID NO: 430
  clone of aptamer PDGF-BB  ARX36.SCK.F12.M13F

SEQ ID NO: 432 - DNA, 82 bp, Artificial
  clone of aptamer PDGF-BB  ARX36.SCK.A07.M13F
gggagaggagagaacgttctacgtgatggctgtgaatgaggtagttcgaatacgctgtcgatcgatcgatcgatgaagggcg

SEQ ID NO: 433 - DNA, 81 bp, Artificial
  clone of aptamer PDGF-BB  ARX36.SCK.C12.M13F
gggagaggagagaacgttctacgtgaaatcaaggttgttaatttggggaatcgctgtcgatcgatcgatcgatgaagggcg

SEQ ID NO: 434 - DNA, 82 bp, Artificial
  clone of aptamer PDGF-BB  ARX36.SCK.B07.M13F
gggagaggagagaacgttctacgtataaggccgtaaccgggtagcgagtggtcgctgtcgatcgatcgatcgatgaagggcg

SEQ ID NO: 435 - DNA, 82 bp, Artificial
  clone of aptamer PDGF-BB  ARX36.SCK.A09.M13F
gggagaggagagaacgttntacgtgggcgaaggagctgcgggcgttgnagtttgctgtcgatcgatcgatcgatgaagggcg

SEQ ID NO: 436 - DNA, 82 bp, Artificial
  clone of aptamer PDGF-BB  ARX36.SCK.A11.M13F
gggagaggagagaacgttctacgtcatcctagtctgagatcggattttcttgcgctgtcgatcgatcgatcgatgaagggcg

SEQ ID NO: 437 - DNA, 82 bp, Artificial
  clone of aptamer PDGF-BB  ARX36.SCK.C09.M13F
gggagaggagagaacgttctacgtttgcgagtgtggtcgacgctgaatgcggcgctgtcgatcgatcgatcgatgaagggcg

SEQ ID NO: 438 - DNA, 82 bp, Artificial
  clone of aptamer PDGF-BB  ARX36.SCK.A08.M13F
gggagaggagagaacgttctacggattgatagggattgagatgaggtcttgtcgctgtcgatcgatcgatcgatgaagggcg

SEQ ID NO: 439 - DNA, 81 bp, Artificial
  clone of aptamer PDGF-BB  ARX36.SCK.D07.M13F
gggagaggagagaacgttctacgatgtcgtgttagattacttattgctatctgctgtcgatcgatcgatcgatgaagggcg

SEQ ID NO: 440 - DNA, 82 bp, Artificial
  clone of aptamer PDGF-BB  ARX36.SCK.D08.M13F
gggagaggagagaacgttctacgatgcctggcggaaacggagcctgggatttcgctgtcnatcgatcgatcgatgaagggcg

SEQ ID NO: 441 - DNA, 80 bp, Artificial
  clone of aptamer PDGF-BB  ARX36.SCK.B11.M13F
gggagaggagagaacgttctacgaggatttgacgtgtgtgtgctagagtacgctgtcgatcgatcgatcgatgaagggcg

SEQ ID NO: 442 - DNA, 82 bp, Artificial
  clone of aptamer PDGF-BB  ARX36.SCK.D09.M13F
gggagaggagagaacgttctacgagtattatgcgtcccttgaggatacacggcgctgtcgatcgatcgatcgatgaagggcg

SEQ ID NO: 443 - DNA, 82 bp, Artificial
  clone of aptamer PDGF-BB  ARX36.SCK.B10.M13F
gggagaggagagaacgttctacagggataactgtagcgatgaaagtaaacgatgctgtcgatcgatcgatcgatgaagggcg

SEQ ID NO: 444 - DNA, 81 bp, Artificial
  clone of aptamer PDGF-BB  ARX36.SCK.C10.M13F
gggagaggagagaacgttctacaagaagtgtggccgcagagacgaaatgcacgctgtcgatcgatcgatcgatgaagggcg

SEQ ID NO: 445 - DNA, 83 bp, Artificial
  clone of aptamer PDGF-BB  ARX36.SCK.A10.M13F
gggagaggagagaacgttctacccatatcttccttctttattccgttagttgccgctgtcgatcgatcgatcgatgaagggcg

SEQ ID NO: 446 - DNA, 82 bp, Artificial
  clone of aptamer PDGF-BB  ARX36.SCK.B09.M13F
gggagaggagagaacgttctacctgtgttgatgcttccgtttgagattgccccgctgtcgatcgatcgatcgatgaagggcg

SEQ ID NO: 447 - DNA, 84 bp, Artificial
  clone of aptamer PDGF-BB  ARX36.SCK.B12.M13F
gggagaggagagaacgttctaccngtaaganaanctattttagcccttgnnctgcgctgtcgatcgatcgatcgatgaagggcg

SEQ ID NO: 448 - DNA, 83 bp, Artificial
  clone of aptamer PDGF-BB  ARX36.SCK.C08.M13F
gggagaggagagaacgttctacccttgtcctccaatcctcttttgactcttgccgctgtcgatcgatcgatcgatgaagggcg

SEQ ID NO: 449 - DNA, 82 bp, Artificial
  clone of aptamer PDGF-BB  ARX36.SCK.D12.M13F
gggagaggagagaacgttctacctgattttgtcactggattccgatggctttcgctgtcgatcgatcgatcgatgaagggcg

SEQ ID NO: 450 - DNA, 83 bp, Artificial
  clone of aptamer PDGF-BB  ARX36.SCK.C11.M13F
gggagaggagagaacgttctactgtaataagggatgcgtcaggaacctgtgttcgctgtcgatcgatcgatcgatgaagggcg

SEQ ID NO: 451 - DNA, 81 bp, Artificial
  clone of aptamer PDGF-BB  ARX36.SCK.D11.M13F
gggagaggagagaacgttctactgctttccgggaatttgtttgtttgcttccgctgtcgatcgatcgatcgatgaagggcg

SEQ ID NO: 452 - DNA, 82 bp, Artificial
  clone of aptamer PDGF-BB  ARX36.SCK.C07.M13F
gggagaggagagaacgttctacttcgtcggttgacttttcttcgtgtagtgtcgctgtcgatcgattgatcgatgaagggcg

SEQ ID NO: 453 - DNA, 92 bp, Artificial
  aptamer library template
catcgatcgatcgatcgacagcgnnnnnnnnnnnnnnnnnnnnnnnnnnnnnngtagaacgttctctcctctccctatagtgagtcgtatta

SEQ ID NO: 454 - DNA, 24 bp, Artificial
  PCR 3′-primer
catcgatgctagtcgtaacgatcc

SEQ ID NO: 455 - DNA, 40 bp, Artificial
  PCR 5′-primer
taatacgactcactatagggagaggagagaaacgttctcg

SEQ ID NO: 456 - RNA, 75 nt, Artificial
  rRmY aptamer ARC256 RNA transcription product
gggagaggagagaacguucuacnnnnnnnnnnnnnnnnnnnnnnnnnnnnnncgcugucgaucgaucgaucgaug

SEQ ID NO: 457 - RNA, 11 nt, Artificial
  mN PEG 5′ oligonucleotide
ggngcngcncc

SEQ ID NO: 458 - RNA, 19 nt, Artificial
  mN PEG 3′ oligonucleotide
ggugccnngucguugcucc

SEQ ID NO: 459 - DNA, 75 bp, Artificial
  aptamer library template
gggagaggagagaacgttctacnnnnnnnnnnnnnnnnnnnnnnnnnnnnnncgctgtcgatcgatcgatcgatg

SEQ ID NO: 460 - DNA, 22 bp, Artificial
  PCR primer
gggagaggagagaacgttctac

SEQ ID NO: 461 - DNA, 22 bp, Artificial
  PCR primer
catcgatcgatcgatcgacagc

SEQ ID NO: 462 - RNA, 75 nt, Artificial
  rGmH aptamer ARC256 transcription product
gggngnggngngnncguucuncnnnnnnnnnnnnnnnnnnnnnnnnnnnnnncgcugucgnucgnucgnucgnug

SEQ ID NO: 463 - RNA, 75 nt, Artificial; identical to SEQ ID NO: 462
  r/mGmH aptamer ARC256 transcription product

SEQ ID NO: 464 - DNA, 75 bp, Artificial
  dRmY aptamer ARC256 transcription product
gggagaggagagaacguucuacnnnnnnnnnnnnnnnnnnnnnnnnnnnnnncgcugucgaucgaucgaucgaug

SEQ ID NO: 465 - DNA, 11 bp, Artificial
  dRmY PEG 5′ oligonucleotide
ggagcagcacc

SEQ ID NO: 466 - DNA, 19 bp, Artificial
  dRmY PEG 3′ oligonucleotide
ggugccaagucguugcucc

SEQ ID NO: 467 - DNA, 80 bp, Artificial
  clone of aptamer ARX34P2.B07
gggagaggagagaacgttctacttgctgtgacggacgggcttgagaggctcgctgtcgatcgatcgatcgatgaagggcg

SEQ ID NO: 468 - RNA, 75 nt, Artificial; identical to SEQ ID NO: 456
  rN aptamer ARC256 transcription product

SEQ ID NO: 469 - DNA, 92 bp, Artificial
  DNA template
catcgatgatcgatcgatcgaccnnnnnnnnnnnnnnnnnnnnnnnnnnnnnngtagaacgttctctcctctccctatagtgagtcgtatta

SEQ ID NO: 470 - DNA, 22 bp, Artificial
  PB.118.95.G primer
gggagaggagagaacgttctac

SEQ ID NO: 471 - DNA, 23 bp, Artificial
  PB.118.95.M Primer
catcgatgatcgatcgatcgacc

SEQ ID NO: 472 - DNA, 75 bp, Artificial
  AMX(221)_E1 clone
gggagaggagagaacgttctaccttggtttggcacaggcatacatacgcaggggtcgatcgatcgatcatcgatg

SEQ ID NO: 473 - DNA, 74 bp, Artificial
  AMX(221)_B3 clone
gggagaggagagaacgttctaccttggtttggcacgggcatacatacgcagggtcgatcgatcgatcatcgatg

SEQ ID NO: 474 - DNA, 74 bp, Artificial
  AMX(221)_F11 clone
gggagaggagagaacgttctacggggaggtgggtgggtagtgttgtgtaacggtcgatcgatcgatcatcgatg

SEQ ID NO: 475 - DNA, 75 bp, Artificial
  AMX(221)_C12 clone
gggagaggagagaacgttctactggcagggcattgagtaagggtgttggtgtggtcgatcgatcgatcatcgatg

SEQ ID NO: 476 - DNA, 75 bp, Artificial
  AMX(221)_E9 clone
gggagaggagagaacgttctacggatggtatcgctgtgctgattgggtgccaggtcgatcgatcgatcatcgatg

SEQ ID NO: 477 - DNA, 73 bp, Artificial
  AMX(221)_A9 clone
gggagaggagagaacgttctacaggagtgcgatgggatcaggtgcgtgcgggtcgatcgatcgatcatcgatg

SEQ ID NO: 478 - DNA, 75 bp, Artificial
  AMX(221)_E8 clone
gggagaggagagaacgttctacatccaccagcccggacatggcttgcacgatggtcgatcgatcgatcatcgatg

SEQ ID NO: 479 - DNA, 74 bp, Artificial
  AMX(221)_C11 clone
gggagaggagagaacgttctacagcaggagagtgtgtgtggcagggagatgggtcgatcgatcgatcatcgatg

SEQ ID NO: 480 - DNA, 75 bp, Artificial
  AMX(221)_H11 clone
gggagaggagagaacgttctacagggtggaaggatgnggtactcnnggcgtgggtcgatcgatcgatcatcgatg

SEQ ID NO: 481 - DNA, 75 bp, Artificial
  AMX(221)_A11 clone
gggagaggagagaacgttctacagataggatggcaaagggggtgtgcaggcaggtcgatcgatcgatcatcgatg

SEQ ID NO: 482 - DNA, 75 bp, Artificial
  AMX(221)_F12 clone
gggagaggagagaacgttctactgaccacggggtatggttactggtttctgaggtcgatcgatcgatcatcgatg

SEQ ID NO: 483 - DNA, 76 bp, Artificial
  AMX(221)_E11 clone
gggagaggagagaacgttctacatgctgcaatcgagaggggggcagtccacgaggtcgatcgatcgatcatcgatg

SEQ ID NO: 484 - DNA, 75 bp, Artificial
  AMX(221)_C9 clone
gggagaggagagaacgttctacagggcgcttatgcaattcaccggaggcaagggtcgatcgatcgatcatcgatg

SEQ ID NO: 485 - DNA, 75 bp, Artificial
  AMX(221)_B1 clone
gggagaggagagaacgttctacgtagggaggatgggtggggataggtgtgcgggtcgatcgatcgatcatcgatg

SEQ ID NO: 486 - DNA, 76 bp, Artificial
  AMX(221)_B4 clone
gggagaggagagaacgttctacaatggtgtgtgatttgaggggagggtggttgggtcgatcgatcgatcatcgatg

SEQ ID NO: 487 - DNA, 75 bp, Artificial
  AMX(221)_F3 clone
gggagaggagagaacgttctacgatggaggaggagtacaggataggctggatggtcgatcgatcgatcatcgatg

SEQ ID NO: 488 - DNA, 75 bp, Artificial
  AMX(221)_G1 clone
gggagaggagagaacgttctacttgttgttgtgtgagtgagtaggctggctgggtcgatcgatcgatcatcgatg

SEQ ID NO: 489 - DNA, 72 bp, Artificial
  AMX(221)_A6 clone
gggagaggagagaacgttctacgtttgcggtcaggatggggtggtgggaggtcgatcgatcgatcatcgatg

SEQ ID NO: 490 - DNA, 73 bp, Artificial
  AMX(221)_A5 clone
gggagaggagagaacgttctacttgtggcaggctgcgtacaggagcagatggtcgatcgatcgatcatcgatg

SEQ ID NO: 491 - DNA, 75 bp, Artificial
  AMX(221)_E6 clone
gggagaggagagaacgttctacgttgtgataggttgtgtgagatggtgtgccggtcgatcgatcgatcatcgatg

SEQ ID NO: 492 - DNA, 73 bp, Artificial
  AMX(221)_D1 clone
gggagaggagagaacgttctacatgtgcaaccaggagcagtaacaggacaggtcgatcgatcgatcatcgatg

SEQ ID NO: 493 - DNA, 74 bp, Artificial
  AMX(221)_H6 clone
gggagaggagagaacgttctacggtttgggtgttggatgggcggttgggagggtcgatcgatcgatcatcgatg

SEQ ID NO: 494 - DNA, 75 bp, Artificial
  AMX(221)_F4 clone
gggagaggagagaacgttctacgggttggacagagagaaggatgagtacgtgggtcgatcgatcgatcatcgatg

SEQ ID NO: 495 - DNA, 75 bp, Artificial
  AMX(221)_D4 clone
gggagaggagagaacgttctacggtaggtgctgggtgcgtaatggcatcgatggtcgatcgatcgatcatcgatg

SEQ ID NO: 496 - DNA, 75 bp, Artificial
  AMX(221)_A4 clone
gggagaggagagaacgttctacgggtgtgtttggtgcaagagtatttgtgcgggtcgatcgatcgatcatcgatg

SEQ ID NO: 497 - DNA, 75 bp, Artificial
  AMX(221)_H4 clone
gggagaggagagaacgttctacagtgtgcgcttggtaatggtggttggagtaggtcgatcgatcgatcatcgatg

SEQ ID NO: 498 - DNA, 74 bp, Artificial
  AMX(221)_C1 clone
gggagaggagagaacgttctactggtagggatgtgcgtagagttgtcgtgtggtcgatcgatcgatcatcgatg

SEQ ID NO: 499 - DNA, 73 bp, Artificial
  AMX(221)_C2 clone
gggagaggagagaacgttctacaacacatctggccatgtcagtcgaggatggtcgatcgatcgatcatcgatg

SEQ ID NO: 500 - DNA, 74 bp, Artificial
  AMX(221)_A1 clone
gggagaggagagaacgttctacacatgccgtgcacccaccacatatccacaggtcgatcgatcgatcatcgatg

SEQ ID NO: 501 - DNA, 74 bp, Artificial
  AMX(221)_F6 clone
gggagaggagagaacgttctacatgcacaacagcacacacgtggcatcgatggtcgatcgatcgatcatcgatg

SEQ ID NO: 502 - DNA, 75 bp, Artificial
  ARC520 sythetic dRmY pool
gggagaggagagaacguucuacnnnnnnnnnnnnnnnnnnnnnnnnnnnnnnggucgaucgaucgaucaucgaug

SEQ ID NO: 503 - DNA, 22 bp, Artificial
  KMT.108.59.B primer
catcgatgatcgatcgatcgac

SEQ ID NO: 504 - DNA, 39 bp, Artificial
  KMT.108.59.A primer
taatacgactcactatagggagaggagagaacgttctac

SEQ ID NO: 505 - DNA, 75 bp, Artificial
  AMX(192)_B5 clone
gggagaggagagaacgttctacgggggtcgtgggagtaagggggtgtaggtaggtcgatcgatcgatcatcgatg

SEQ ID NO: 506 - DNA, 75 bp, Artificial
  AMX(192)_G10 clone
gggagaggagagaacgttctacgggtggatgggagggggacaggtaggatggggtcgatcgatcgatcatcgatg

SEQ ID NO: 507 - DNA, 75 bp, Artificial; identical to SEQ ID NO: 505
  AMX(192)_F8 clone

SEQ ID NO: 508 - DNA, 73 bp, Artificial
  AMX(192)_E3 clone
gggagaggagagaacgttctacgggtggctggggcaggggaggtaggtagggtcgatcgatcgatcatcgatg

SEQ ID NO: 509 - DNA, 75 bp, Artificial
  AMX(192)_G11 clone
gggagaggagagaacgttctacgggtggatgggagggggacaggcaggatggggtcgatcgatcgatcatcgatg

SEQ ID NO: 510 - DNA, 75 bp, Artificial
  AMX(192)_G9 clone
gggagaggagagaacgttctacgggtggttgggaagggggatggaggtatggggtcgatcgatcgatcatcgatg

SEQ ID NO: 511 - DNA, 72 bp, Artificial; identical to SEQ ID NO: 489
  AMX(192)_A5 clone

SEQ ID NO: 512 - DNA, 75 bp, Artificial
  AMX(192)_F3 clone
gggagaggagagaacgttctacgggcggttggggtcggggaggatggtacagggtcgatcgatcgatcatcgatg

SEQ ID NO: 513 - DNA, 75 bp, Artificial
  AMX(192)_D11 clone
gggagaggagagaacgttctacggggaggagggtggggtagcaggtgtggcaggtcgatcgatcgatcatcgatg

SEQ ID NO: 514 - DNA, 74 bp, Artificial
  AMX(192)_F11 clone
gggagaggagagaacgttctactcgggtgggggggcagcaaggtagctgtaggtcgatcgatcgatcatcgatg

SEQ ID NO: 515 - DNA, 75 bp, Artificial; identical to SEQ ID NO: 505
  AMX(216)_A7 Clone

SEQ ID NO: 516 - DNA, 75 bp, Artificial
  AMX(216)_D5 clone
gggagaggagagaacgttctacgatgggcggatggtgggaggatgggcaataggtcgatcgatcgatcatcgatg

SEQ ID NO: 517 - DNA, 75 bp, Artificial; identical to SEQ ID NO: 505
  AMX(216)_B7 clone

SEQ ID NO: 518 - DNA, 76 bp, Artificial
  AMX(216)_H1 clone
gggagaggagagaacgttctacgggggtcgtgggagtaaggggggtgtaggtaggtcgatcgatcgatcatcgatg

SEQ ID NO: 519 - DNA, 75 bp, Artificial
  AMX(216)_D12 clone
gggagaggagagaacnttctaccggggtcgtgggagtaagggggtgtaggtaggtcnatcnatcnatcntcnatg

SEQ ID NO: 520 - DNA, 75 bp, Artificial
  AMX(216)_G2 clone
gggagaggagagaacgttctacgggggtcgtgggagaaagggggtgtaggtaggtcgatcgatcgatcatcgatg

SEQ ID NO: 521 - DNA, 75 bp, Artificial
  AMX(216)_G4 clone
gggagaggagagaacgttctacgggcggtgggggtcggggaggatggtacagggtcgatcgatcgatcatcgatg

SEQ ID NO: 522 - DNA, 73 bp, Artificial
  AMX(216)_A6 clone
gggagaggagagaacgttctacgggtggttggggcaggggaggtaggtagggtcgatcgatcgatcatcgatg

SEQ ID NO: 523 - DNA, 32 bp, Artificial
  ARC872 Minimer of AMX(192)_E3
gggcgguuggggucggggaggaugguacaggg

SEQ ID NO: 524 - DNA, 30 bp, Artificial
  ARC873 Minimer of AMX(192)_F3
ggguggcuggggcaggggagguagguaggg